(19) United States
(12) Patent Application Publication
(10) Pub. No.: US 2021/0260022 A1
(43) Pub. Date: Aug. 26, 2021

(54) SYNERGY FOR INCREASING ENERGY EXPENDITURE AND INSULIN SENSITIVITY

(71) Applicant: The Board of Supervisors of Louisiana State University and Agricultural and Mechanical College, Baton Rouge, LA (US)

(72) Inventors: Frank Greenway, Baton Rouge, LA (US); Ann Coulter, Baton Rouge, LA (US); Candida Rebello, Baton Rouge, LA (US)

(21) Appl. No.: 17/245,538

(22) Filed: Apr. 30, 2021

Related U.S. Application Data
(63) Continuation-in-part of application No. PCT/US2019/059397, filed on Nov. 1, 2019.
(60) Provisional application No. 62/754,045, filed on Nov. 1, 2018.

(51) Int. Cl. A61K 31/352; A61K 31/047; A61K 31/015; A61K 31/01; A61K 9/00; A61P 3/04

(52) U.S. Cl. CPC A61K 31/352; A61K 31/047; A61P 3/04; A61K 31/01; A61K 9/0014; A61K 31/015

(57) ABSTRACT

The invention is directed to compositions and methods for treating metabolic disorders.

This application is a Continuation-In-Part part of International Application No. PCT/US2019/059397 filed on Nov. 1, 2019, which claims priority from U.S. Provisional Application 62/754,045 filed on Nov. 1, 2018, the contents of which are hereby incorporated by reference in their entireties.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

The approved obesity drugs for long-term use are not very successful in the market due to their high prices. The approved medications for the treatment of obesity reduce food intake.

### SUMMARY OF THE INVENTION

Aspects of the invention are directed towards compositions and methods for treating metabolic disorders.

Embodiments comprise a composition comprising a therapeutically effective amount of at least one flavonoid and a therapeutically effective amount of at least one carotenoid.

For example, the composition can comprise about 150 mg to about 900 mg of at least one flavonoid.

In embodiments, the flavonoid is naringenin.

For example, the composition can comprise about 1 mg to about 12 mg of at least one carotenoid.

In embodiments, the at least one carotenoid is selected from the group consisting of beta carotene, lycopene, or lutein. For example, the carotenoid is beta carotene.

In embodiments, the composition can further a sufficient amount of a pharmaceutically acceptable carrier.

In embodiments, the composition can further comprise one or more additional active agents. For example, the additional active agent can comprise an anti-obesity agent.

In embodiments, the composition is provided as an injectable solution, an oral dose, a topical cream, a topical gel, or a medical food.

In embodiments, the composition is for use in treating a subject afflicted with a metabolic disorder.

In embodiments, the composition is for use in local fat reduction.

In embodiments, the composition is for use in converting white fat to brown fat in a subject.

Aspects of the invention are also directed towards a method for treating a subject afflicted with a metabolic disorder. For example, the metabolic disorder comprises obesity, insulin resistance, type 2 diabetes, metabolic syndrome, and non-alcoholic steohepatis.

In embodiments, the method comprises administering to the subject a therapeutically effective amount of the composition described herein. For example, the composition comprises a therapeutically effective amount of naringenin and a therapeutically effective amount of at least one carotenoid.

In embodiments, the composition is administered as a topical cream, administered as a topic gel, administered as a medical food, or administered as an injectable.

In embodiments, the topical cream is administered locally.

Still further, aspects of the invention are drawn to a method of converting white fat to brown fat in a subject.

In embodiments, the method comprising administering to the subject a therapeutically effective amount of the composition described herein. For example, the composition comprises a therapeutically effective amount of naringenin and a therapeutically effective amount of at least one carotenoid.

In embodiments, the composition is administered as a topical cream, administered as a topic gel, administered as a medical food, administered as an oral dose, or administered as an injectable.

In embodiments, the topical cream is administered locally.

Further, aspects of the invention are drawn to a method for local fat reduction,

the method comprising administering to a site on a subject a therapeutically effective amount of the composition of any one of claims 1-9.

In embodiments, the method comprising administering to the subject a therapeutically effective amount of the composition described herein. For example, the composition comprises a therapeutically effective amount of naringenin and a therapeutically effective amount of at least one carotenoid.

In embodiments, the composition is administered as a topical cream, administered as a topic gel, administered as a medical food, administered as an oral dose, or administered as an injectable.

In embodiments, the topical cream is administered locally.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows naringenin induces expression of genes for energy expenditure and glucose utilization.

FIG. 2 shows human adipocyte cultures were treated with naringenin for 0, 3 or 7d. Total protein was isolated and analyzed by Western Blotting.

FIG. 3 shows oxygen consumption rate in human adipocytes after naringenin treatment

FIG. 4 shows naringenin and beta carotene treatment of human subcutaneous adipocytes from obese donors

FIG. 5 shows other carotenoids, lutein and lycopene, act synergistically with naringenin extract to induce UCP1 and GLUT4 expression in human adipocytes Differentiated human adipocytes were treated for seven days with 8 μM naringenin extract (Nar) alone or in combination with 2 μM carotenoid. There was no effect of carotenoid alone. Carotenoids tested were β-carotene (βCar), Lycopene (lyco) and lutein.

FIG. 6 shows treatment of HepG2 cells with beta carotene and naringenin induces expression of liver fat oxidation genes In a cell culture model for fatty liver using human hepatoma HepG2 cells maintained in 0.5 mM oleic acid and treated with naringenin for 24 h, there was a trend toward enhanced expression of fat oxidation genes after addition of beta carotene. These studies are ongoing to evaluate synergy.

FIG. 7 shows induction of UCP1 expression by naringenin in human adipocytes requires PPARα or PPARγ activity. Human white adipocytes were treated for 2d with naringenin in the presence of inhibitors of metabolic pathways to determine the mechanism of action for stimulating fat oxidation and thermogenic energy expenditure. Induction of UCP1 and CPT-1b was abolished by inhibitors of PPARα (GW6471), or PPARγ (GW9661) or PI3 Kinase (LY294002), partially reduced by an inhibitor of protein kinase A (H89) and unaffected by a protein kinase G inhibitor (Biolog C013).

FIG. 8 shows other combinations with naringenin do not have synergy. Human adipocytes were treated with 5 μM naringenin or with naringenin in combination with other botanical compounds to evaluate synergy in inducing UCP1 mRNA expression. A range of other botanical compounds such as Ecalyptol, iicillin and OEA (oleoylethanolamide) and did not synergize with naringenin to increase UCP1 mRNA levels over those of naringenin alone.

FIG. 9 shows pear-shaped woman used 0.5% aminophylline cream on her hips and thighs during weight loss.

FIG. 10 shows a schematic of cellulite, which is the bumpy skin such as on the thighs. See, for example, Skin Smoother within 2 weeks Girth Reduction comes next Ronsard, N. Cellulite. Beauty & Healthy Publishing, New York, 1973.

FIG. 11 shows results of 0.5% Aminophylline Cream used in 12 patients. Smaller thighs, no weight loss, fat redistribution. See Greenway F L et al. Obesity Research. 1995; 3(Suppl. 4): 561S-567S

FIG. 12 shows waist reduction with 0.6% Aminophylline cream. See Caruso M K et al. Diabetes Obes Metab. 2007; 9(3):300-3.

FIG. 13 shows weight loss is not a straight line and settles at a new level after about 6 months.

FIG. 14 shows weight loss due to orange juice extract. See Cardile V et al. Naural Products Research. 2015; 29(23):2256-60.

FIG. 15 is a schematic of the concept of re-engineering fat cells to burn fat.

FIG. 16 shows an image of brown adipose tissue. Brown Adipose Tissue is a specialized fat tissue in the upper back (interscapular) region that keeps hibernating animals warm by producing heat. A unique feature is the expression of Uncoupling Protein 1 in mitochondria.

FIG. 17 shows an image of brown fat tissue in humans. Brown fat tissue in humans helps infants maintain body temperature, and is almost undetectable by adulthood. UCP1 causes fat cells to use glucose and fatty acids as fuel to produce heat. Adults have very little BAT and have diminished response to cold. UCP1 levels get lower with greater body fat (BMI) and increasing age.

FIG. 18 shows BAT is activated by cold exposure. Cold receptors are on fat cells and are in sensory nerve endings in skin. After cold stimulation, the nervous system activates UCP1 expression and heat production in BAT. Cold exposed adults have slightly more UCP1.

FIG. 19 shows an advertisement for a cancer drug called Roscovitine with toxic side effects. So far, drugs that induce UCP1 in white fat cells have side effects. Is there a better way to induce UCP1 and fat oxidation in white adipocytes? For example, better compositions and methods that are less painful than cold exposure, fasting, or toxic side effects like vomiting?

FIG. 20 is a schematic of PPARγ, which is an activator of white fat genes and fat storage.

FIG. 21 shows a schematic of the activity of Roscovitine.

FIG. 22 shows a schematic of a better way to induce UCP1 expression.

FIG. 23 shows images of pre-adipocytes and adipocytes. In our preclinical research lab, we tested orange extract on human abdominal fat cells from liposuction of overweight/obese women.

FIG. 24 shows 7 day treatment with orange extract increased UCP1 expression over 7-fold in human fat cells.

FIG. 25 shows treatment with orange extract and β-carotene increases UCP1 expression over 12-fold.

FIG. 26 shows 7 day treatment induces expression of cold-sensing TrpM8 receptors 4.5-fold.

FIG. 27 shows expression of mitochondrial fat oxidation genes.

FIG. 28 shows that studies in mice with ovaries removed surgically, a model for low estrogen levels, showed that orange extract prevents weight gain, lowers fasting glucose and insulin, and maintains muscle mass.

FIG. 29 shows a schematic of effect of citrus extract and vitamin A on a cell.

FIG. 30 shows change in resting metabolic rate following eight weeks of naringenin treatment. Change from baseline in the five-hour resting metabolic rate following eight weeks of naringenin treatment at 150 mg three times daily.

FIG. 31 shows qRT-PCR in human adipocytes treated with naringenin and inhibitors. qRT-PCR assays for mRNA expression conducted in duplicates following naringenin treatment of hADSC for two days compared to naringenin treatment+inhibitors and untreated hADSC (Control): uncoupling protein 1 (UCP1) and carnitine palmitoyltransferase 1β (CPT1β), The results are shown as mean±SEM. Naringenin increased UCP1 and CPT1β mRNA induction compared to control (p<0.001). PPARα and PPARγ inhibition reduced UCP1 and CPT1β mRNA expression (p<0.001). Each experiment was conducted with three biological replicates and four technical replicates.

FIG. 32 shows anthropometric measurements and vital signs obtained at baseline, week 4 and week 8 clinic visits.

FIG. 33 shows serum concentrations of chemistry panel markers at baseline and after eight weeks.

FIG. 34 shows results of the complete blood count (CBC) obtained at baseline and week 8.

FIG. 35 shows Naringenin extract and β-carotene increase PGC-1α protein levels.

FIG. 36 shows Naringenin extract and β-carotene increase adiponectin protein levels.

FIG. 37 shows Naringenin extract and β-carotene increase PPARα protein levels.

FIG. 38 shows Naringenin extract and β-carotene increase NAMPT protein levels.

FIG. 39 shows a schematic of Naringenin and β-carotene in a cell. RXR: retinoic acid receptor, LXR liver X receptor, CKMT mitochondrial creatine kinase, PKA protein kinase A, PTHR parathyroid hormone receptor, NPR1 natriuretic peptide receptor, B1AR beta-1 adrenergic receptor, TG triglyceride, HSL hormone sensitive lipase, MGLL monoglyceride lipase, PM20D1 peptidase M20 domain containing, ANGPTL4 angiopoietin like 4.

FIG. 40 shows Naringenin extract and β-carotene treatment induces synergistic increases in expression of metabolic adipocyte genes. Panel A shows a graph and panel B shows a Western blot.

FIG. 41 shows graphs and Western blots of induction of proteins (PPARα in panel a, PPARγ in panel b, PGC1α in panel c, and NAMPT in panel d.) occurs in absence of mRNA increases.

FIG. 42 shows general gene ontology analysis in panel a, low expression receptors (panel b) receptors βAR: beta adrenergic receptor; TGR5: bile acid receptor; TRPM8: transient receptor potential melastatin 8; MC1R: melanocortin 1 receptor; ADORA1, ADORA2B: adenosine receptors A1 and A2B; and high expression receptors (panel c) NPR1, NPR2: atrial natriuretic peptide receptors; GPER1: G-protein coupled estrogen receptor 1; PTHR: parathyroid receptor 1; GH: growth hormone receptor; Data are expressed in mean transcript numbers for each gene per total kilobases read.

FIG. 43 (panels a, b, and c) shows graphs of RT-PCR validation of NRBC gene induction

FIG. 44 shows a graph of after 7 days of treatment with vehicle or NR+BC. NPR1, NPR2: atrial natriuretic peptide receptors GPER1: G-protein coupled estrogen receptor 1 PTHR: parathyroid receptor 1 GH: growth hormone receptor Data are expressed in mean transcript numbers for each gene per total kilobases read. * p≤0.02 cAMP: 8-Cpt-cAMP 200 μM; ANP: atrial natriuretic peptide 0.1 μM; PTH: parathyroid hormone (1-34) 1 μM; isoprot: isoproterenol 1 μM; dobut: dobutamine 1 μM; estradiol 1 μM; GH: growth hormone 250 ng/ml; ACTH: adrenocorticotropin hormone 1 μM; CDCA: chenodeoxycholic acid 30 μM; adenosine 1 μM; menthol 100 μM.

FIG. 45 shows lipolysis is higher in adipocytes after nar+BC treatment. Acute agonist-stimulated glycerol release in untreated and NRBC-treated adipocytes. After 7d treatment with vehicle or NRBC, cells were exposed to receptor agonists dissolved in KRB with 1% BSA for 4.5 hours. Supernatants were removed for measurement of glycerol. Data are presented as mean±SEM from experiments using cells from four different donors with BMIs ranging from 27 to 36, each with at least 6 replicates. * p≤0.02 cAMP 8-Cpt-cAMP 200 μM, ANP atrial natriuretic peptide 0.1 μM, PTH parathyroid hormone (1-34) 1 μM, isoprotisoproterenol 1 μM, dobut dobutamine 1 μM, estradiol 1 μM, GH growth hormone 250 ng/ml, ACTH adrenocorticotropin hormone CDCA chenodeoxycholic acid 30 μM, adenosine 1 μM, menthol 100 μM.

### DETAILED DESCRIPTION OF THE INVENTION

### Abbreviations and Definitions

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the invention in any appropriate manner.

The singular forms “a,” “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be non-limiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises,” “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of When the term “about” is used in conjunction with a numerical range, it modifies that range by extending the boundaries above and below the numerical values set forth. For example, the term “about” is used herein to modify a numerical value above and below the stated value by a variance of 20 percent up or down (higher or lower).

The term “consisting essentially of” can refer to a composition, whose only active ingredient is the indicated active ingredient, however, other compounds can be included which are for stabilizing, preserving, etc. the formulation, but are not involved directly in the therapeutic effect of the indicated active ingredient. In some embodiments, the term “consisting essentially of” can refer to components which facilitate the release of the active ingredient. For example, a composition described herein can consist essentially of naringenin and a carotenoid, such as beta carotene, lycopein, or luteine. Such composition can also other compounds which are for stabilizing, preserving, or facilitating the release of the formulation, but are not involved directly in the therapeutic effect of the indicated active ingredient.

The term “consisting” can refer to a composition, which contains the active ingredient and a pharmaceutically acceptable carrier or excipient. For example, a composition described herein can consist of naringenin and a carotenoid, such as beta carotene, lycopein, or luteine, together with a pharmaceutically acceptable carrier or excipient.

Various exemplary embodiments of the invention described herein can comprise compositions and methods for treating a subject afflicted with a metabolic disorder or preventing the onset of a metabolic disorder, such as obesity, insulin resistance, type 2 diabetes, and non-alcoholic steohepatis. Other exemplary embodiments can comprise compositions and methods for converting white fat to brown fat in a subject, and for local fat reduction. For example, embodiments can comprise administering to the subject a composition comprising a therapeutically effective amount of naringenin and at least one carotenoid, such as beta carotene, lycopene, or lutein. In embodiments, the composition can be applied to the skin of a subject as a topical cream or is ingested by the subject as a food.

In embodiments, the composition comprises a therapeutically effective amount of naringenin and a therapeutically effective amount of at least one carotenoid, such as beta carotene, lycopein, or luteine, and can further comprise one or more additional active agents. For example, the one or more additional active agents can comprise an anti-obesity agent, such as phentermine.

Those skilled in the art will recognize, or be able to ascertain, using no more than routine experimentation, numerous equivalents to the specific compositions and methods described herein. Such equivalents are considered to be within the scope of this invention, and are covered by the following claims.

Synergistic Compositions

Aspects of the disclosure are drawn to a composition comprising a therapeutically effective amount of naringenin and a therapeutically effective amount of at least one beta carotenoid. Referring to the examples, such compositions show a synergistic effect on UCP1 expression, for example, and can be useful for preventing or treating a metabolic disorder, local fat reduction, and/or converting white fat to brown fat in a subject.

As used herein, a “therapeutic composition” can refer to a composition comprising one or more active ingredient(s) required to cause an effect when an effective amount of the composition is administered to a subject in need thereof. For example, the effect can be prevention or treatment of a metabolic disorder, local fat reduction, and/or converting white fat to brown fat in a subject.

In embodiments, the composition can comprise a therapeutically effective amount of naringenin. Naringenin is a flavanone, a type of flavonoid, that is flavorless and colorless. It is the predominant flavanone in grapefruit, and is found in a variety of fruits and herbs. Naringenin has the skeleton structure of a flavanone with three hydroxy groups at the 4′, 5, and 7 carbons. It can be found both in the aglycol form, naringenin, or in its glycosidic form, naringin, which has the addition of the disaccharide neohesperidose attached via a glycosidic linkage at carbon 7. Naringenin and its glycoside has been found in a variety of herbs and fruits, including grapefruit, bergamot, sour orange, tart cherries, tomatoes, cocoa, Greek oregano, water mint, drynaria as well as in beans. Ratios of naringenin to naringin vary among sources, as do enantiomeric ratios. The isolation methods of naringenin are well known in the art. See, for example, Wang, Chung-Yi, et al. “Quality changes in high hydrostatic pressure and thermal pasteurized grapefruit juice during cold storage.” Journal of food science and technology 55.12 (2018): 5115-5122.

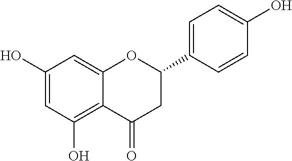 [SMILES: O=C1C[C@@H](c2ccc(O)cc2)Oc2cc(O)cc(O)c21]

### Structure of Naringenin

Embodiments can further comprise a therapeutically effective amount of at least one carotenoid, such as beta-carotene, leutine, or lycopene. Carotenoids are yellow, orange, and red organic pigments that are produced by plants and algae, as well as several bacteria and fungi. [here are over 1,100 known carotenoids which can be categorized into two classes, xanthophylls (which contain oxygen) and carotenes (which are purely hydrocarbons, and contain no oxygen). Carotenoids can be derivatives of tetraterpenes, meaning that they are produced from 8 isoprene molecules and contain 40 carbon atoms. Carotenoids absorb wavelengths ranging from 400-550 nanometers (violet to green light). This causes the compounds to be deeply colored yellow, orange, or red. The structure of carotenoids imparts biological abilities, including photosynthesis, photoprotection, plant coloration, and cell signaling. The structure of the carotenoid is a polyene chain consisting of 9-11 double bonds and, without wishing to be bound by theory, terminating in rings. This structure of conjugated double bonds leads to a high reducing potential, or the ability to transfer electrons throughout the molecule.

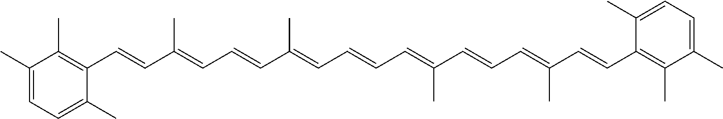

Structure of a Carotenoid: Polyene Tail with Double Bonds, and Terminal Rings (without Wishing to be Bound by Theory)
The isolation methods of carotenoids are well known in the art. See, for example, Vieira, Flavia A., and Sónia PM Ventura. “Efficient Extraction of Carotenoids from Sargassum muticum Using Aqueous Solutions of Tween 20.” Marine drugs 17.5 (2019): 310.

Lutein is a xanthophyll and one of 600 known naturally occurring carotenoids. Lutein is synthesized only by plants and like other xanthophylls is found in high quantities in green leafy vegetables such as spinach, kale and yellow carrots.

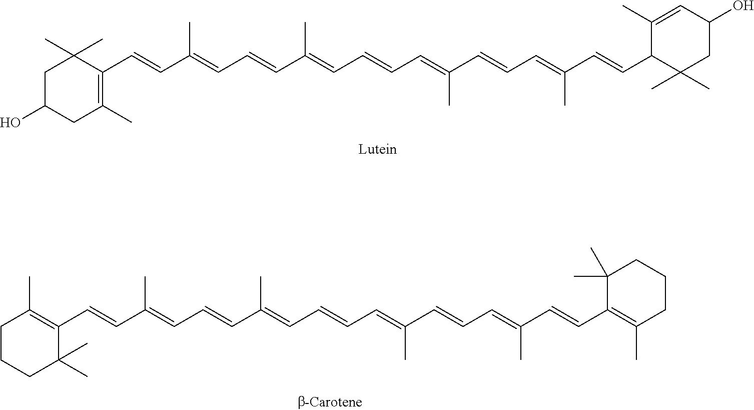

Lycopene is a bright red carotenoid hydrocarbon found in tomatoes and other red fruits and vegetables, such as red carrots, watermelons, gac melons, and papayas, but it is not present in strawberries or cherries. Although lycopene is chemically a carotene, it has no vitamin A activity. Foods that are not red can also contain lycopene, such as asparagus and parsley.

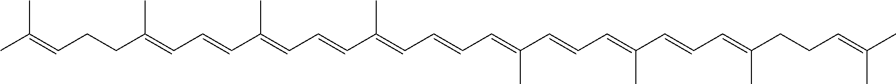 [SMILES: CC(C)=CCC/C(C)=C/C=C/C(C)=C/C=C/C(C)=C/C=C/C=C(C)/C=C/C=C(C)/C=C/C=C(\C)CCC=C(C)C]

### Skeletal Formula of all-Trans Lycopene

β-Carotene is an organic, strongly colored red-orange pigment abundant in plants and fruits. It is a member of the carotenes, which are terpenoids (isoprenoids), synthesized biochemically from eight isoprene units and thus having 40 carbons. Among the carotenes, β-carotene is distinguished by having beta-rings at both ends of the molecule. β-Carotene is biosynthesized from geranylgeranyl pyrophosphate. β-Carotene is the most common form of carotene in plants. In nature, β-carotene is a precursor (inactive form) to vitamin A via the action of beta-carotene 15,15′-monooxygenase.

As described herein, aspects of the disclosure are drawn to compositions comprising therapeutically effective amounts of naringenin and therapeutically effective amounts of a carotenoid. Further, aspects of the disclosure are drawn to methods comprising therapeutically effective amounts of a composition comprising naringenin and a carotenoid. The term “therapeutically effective amount” can refer to those amounts that, when administered to a subject in view of the nature and severity of that subject's disease or condition, will have a therapeutic effect, e.g., an amount which will cure, prevent, inhibit, reduce or at least partially arrest or partially prevent a target disease or condition. In some embodiments, the term “therapeutically effective amount” or “effective amount” can refer to an amount of a therapeutic agent that when administered alone or in combination with an additional therapeutic agent to a cell, tissue, or subject is effective to prevent or ameliorate the disease or condition, such as a metabolic disorder, or the progression of the disease or condition. A therapeutically effective dose further refers to that amount of the therapeutic agent sufficient to result in amelioration of symptoms, e.g., treatment, healing, prevention, reduction or amelioration of the relevant medical condition, such as local fat reduction, or an increase in rate of treatment, healing, prevention or amelioration of such conditions. When applied to an individual active ingredient administered alone, a therapeutically effective dose can refer to that ingredient alone. When applied to a combination, a therapeutically effective dose can refer to combined amounts of the active ingredients that result in the therapeutic effect, whether administered in combination, serially or simultaneously.

The dosage can vary depending upon a number of factors known to those of ordinary skill in the art. For example, the dose(s) can vary depending upon the identity, age, sex, health, weight, size, and condition of the subject or sample being treated, and the nature and extent of the condition. The dosage can further depend on the effect which is desired by the practitioner, pharmacodynamic characteristics of the active ingredient and its mode and route of administration; time of administration of active ingredient; kind of concurrent treatment, frequency of treatment and the desired effect; and rate of excretion. These amounts can be readily determined by the skilled artisan.

In some embodiments, the therapeutically effective amount of β-carotene comprises less than about 1 mg/day, about 1 mg/day, about 2 mg/day, about 3 mg/day, about 4 mg/day, about 5 mg/day, about 6 mg/day, about 7 mg/day, about 8 mg/day, about 9 mg/day, about 10 mg/day, about 11 mg/day, about 12 mg/day, about 13 mg/day, about 14 mg/day, about 15 mg/day, about 16 mg/day, about 17 mg/day, about 18 mg/day, about 19 mg/day, about 20 mg/day, about 21 mg/day, about 22 mg/day, about 23 mg/day, about 24 mg/day, about 25 mg/day, or greater than 25 mg/day. For example, the therapeutically effective amount of β-carotene comprises about 12 mg/day or less than about 12 mg/day.

In some embodiments, the therapeutically effective amount of naringenin is about 10 mg/day, about 50 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1100 mg/day, about 1200 mg/day, about 1300 mg/day, about 1400 mg/day, about 1500 mg/day, about 1600 mg/day, about 1700 mg/day, about 1800 mg/day, about 1900 mg/day, about 2000 mg/day, about 2500 mg/day, about 3000 mg/day, about 3500 mg/day, about 4000 mg/day, about 4500 mg/day, about 5000 mg/day, or greater than about 5000 mg/day. For example, the therapeutically effective amount of naringenin is between about 150 mg/day to about 900 mg/day.

Compounds, for example naringenin or a carotenoid, can be incorporated into pharmaceutical compositions suitable for administration to a subject. Such compositions can comprise naringenin and/or a carotenoid and a pharmaceutically acceptable carrier or excipient. Thus, in some embodiments, the compounds of the invention are present in a pharmaceutical composition.

A pharmaceutically acceptable carrier can comprise one or more solvents, dispersion medias, coatings, antibacterial and antifungal agents, isotonic and absorption delaying agents, and the like, compatible with pharmaceutical administration to a subject. The use of such media and agents for pharmaceutically active substances is well known in the art. Any conventional media or agent that is compatible with the active compound can be used. Supplementary active compounds can also be incorporated into the compositions.

Any of the therapeutic applications or methods of use described herein can be applied to any subject in need of such therapy, including, for example, a mammal such as a mouse, a rat, a dog, a cat, a cow, a horse, a rabbit, a monkey, a pig, a sheep, a goat, or a human. In some embodiments, the subject is a mouse, rat or human. In some embodiments, the subject is a mouse. In some embodiments, the subject is a rat. In some embodiments, the subject is a human.

A pharmaceutical composition of the invention is formulated to be compatible with its intended route of administration. Examples of routes of administration include parenteral, e.g., intravenous, intradermal, subcutaneous, oral (i.e., capsule or medical food), nasal (e.g., inhalation), transdermal (topical, such as a cream), transmucosal, and rectal administration.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). The composition can be sterile and can be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional ingredient from a previously sterile-filtered solution thereof.

Oral compositions can include an inert diluent or an edible carrier. They can be enclosed in gelatin capsules, compressed into tablets, or prepared as a medical food. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

In embodiments, the composition can be prepared as a medical food, dietary item or food ingredient. The term “dietary item” can include any product that undergoes at least one processing or culinary step prior to distribution and is consumed by a subject. Non-limiting examples of processing and culinary steps include mixing, cooking, baking, heating, chopping, chilling, freezing, packaging, canning, bagging, and storing. Non-limiting examples of dietary items include food products, dietary ingredients, medical foods, functional foods, beverages, dietary supplements, vitamins, minerals, and combinations thereof. Unprocessed, raw, or fresh foods, such as fresh fruits and vegetables, are not included herein within this term.

The term “food ingredient” can refer to any edible substance that is combined is with other edible substances, where the final combination is consumed as a food. The term “medical food” herein is defined by statute in the United States of America, Orphan Drug Act, section 5(b) (21 U.S.C. 360ee (b) (3)), which defines “medical food” as “a food which is formulated to be consumed or administered enterally under the supervision of a physician and which is intended for the specific dietary management of a disease or condition for which distinctive nutritional requirements, based on recognized scientific principles, are established by medical evaluation.”

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art.

Many embodiments of the invention are suitable for topical administration to a subject. Non-limiting examples of such embodiments comprise solutions, lotions, creams, ointments, gels, pastes, sprays, liquids, washes, hydrating agents or solutions, and perfusing agents or solutions. Topical doses of a compositions is higher than those doses if administered orally or intravenously, for example, as getting across the skin often requires a higher dose. Such doses can comprises those that are 2 times, 3 times, 4 times, 5 times, 6 times, 7 times, 8 times, 9 times, 10 times, or greater than 10 times the oral dose. Such doses can comprises those that are 2 times, 3 times, 4 times, 5 times, 6 times, 7 times, 8 times, 9 times, 10 times, or greater than 10 times the intravenous dose.

Compositions as described herein can comprise a synergistic combination of naringenin and at least one carotenoid. The term “combination” can refer to a fixed combination in one dosage unit form, or a kit of parts for the combined administration where a compound and a combination partner (e.g., another drug, also referred to as “therapeutic agent” or “co-agent”) can be administered independently at the same time or separately within time intervals, especially where these time intervals allow that the combination partners show a cooperative, e.g., synergistic effect. The terms “co-administration” or “combined administration” or the like as utilized herein can encompass administration of the selected combination partner to a single subject in need thereof (e.g., a patient), and can include treatment regimens in which the agents are not necessarily administered by the same route of administration or at the same time.

The term “pharmaceutical combination” can refer to a product that results from the mixing or combining of more than one active ingredient and includes both fixed and non-fixed combinations of the active ingredients.

The term “fixed combination” can refer to active ingredients, e.g., a compound and a combination partner, that are both administered to a patient simultaneously in the form of a single entity or dosage. The term “non-fixed combination” can refer to the active ingredients, e.g., a compound and a combination partner, that are both administered to a patient as separate entities simultaneously, concurrently or sequentially with no specific time limits, wherein such administration provides therapeutically effective levels of the two compounds in the body of the patient. The latter also applies to cocktail therapy, e.g., the administration of three or more active ingredients.

Embodiments can be administered alone to a subject, in combination with another pharmaceutical drug, as part of treatment regimen, or a component of a kit. In some embodiments, the other pharmaceutical drug is a drug used to a metabolic disorder or contribute to localized fat reduction. For example, such metabolic conditions can be diabetes, metabolic syndrome, or obesity, and such other pharmaceutical drug can be insulin, amylinomimetic agents, alpha-glucosidase inhibitors, biguanides, dopamine agonists, glucagon-like peptides, meglitinides, sodium glucose transporter 2 inhibitors, sulfonylureas, thiazolidinediones, and dipeptidyl peptidase-4 inhibitors. In some embodiments, the drug comprises regular insulin such as but not limited to Humulin or Novolin, insulin aspart such as but not limited to Novolog or FlexPen; insulin glulisine such as but not limited to Apidra; insulin lispro such as but not limited to Humalog; insulin isophane such as but not limited to Humulin N or Novolin N; insulin degludec such as but not limited to Tresiba; insulin detemir such as but not limited to Levemir; insulin glargine such as but not limited to Lantus; insulin glargine such as but not limited to Toujeo; a combination insulin drug such as but not limited to insulin aspart protamine-insulin aspart, insulin lispro protamine-insulin lispro, human isophane insulin-human insulin regular, insulin dedludec-insulin aspart, NovoLog Mix 70/30, Humalog Mix 75/25, Humalog Mix 50/50, Humalin 70/30, Novolin 70/30, or Ryzodeg; pramlintide such as but not limited to SymlinPen; acarbose such as but not limited to Precose; miglitol such as but not limited to Glyset; metformin such as but not limited to Glucophage, Metformin Hydrochloride ER, Glumetza, Riomet, or Fortamet; a metformin-containing drug such as but not limited to metformin-alogliptin, Kazano, metformin-canagliflozin, Invokamet, metformin-dapagliflozin, Xigduo XR, metformin-empagliflozin, Synjardy, metformin-glipizide, metformin-glyburide, Glucovance, metformin-linagliptin, Jentadueto, metformin-pioglitazone, Actoplus, Actoplus Met, Actoplus Met XR, metformin-repaglinide, PrandiMet, metformin-rosiglitazone, Avandamet, metformin-saxagliptin, Kombiglyze XR, metformin-sitagliptin, Janumet, or Janumet XR; bromocriptine such as but not limited to Parlodel; alogliptin such as but not limited to Nesina; alogliptin-pioglitazone such as but not limited to Oseni; linagliptin such as but not limited to Tradjenta, linagliptin-empagliflozin such as but not limited to Glyzami; saxagliptin such as but not limited to Onglyza; sitagliptin such as but not limited to Januvia; sitagliptin and simvastatin such as but not limited to Juvisync; albiglutide such as but not limited to Tanzeum; dulaglutide such as but not limited to Trulicity; exenatide such as but not limited to Byetta; exenatide extended-release such as but not limited to Bydureon; liraglutide such as but not limited to Victoza; nateglinide such as but not limited to Starlix; repaglinide such as but not limited to Prandin; dapagliflozin such as but not limited to Farxiga; canaglifoxin such as but not limited to Invokana; empaglifozin such as but not limited to Jardiance; empagliflozin-linagliptin such as but not limited to Glyxambi; glimepiride such as but not limited to Amaryl; glimepiride-pioglitazone such as but not limited to Duetact; glimepiride-rosiglitazone such as but not limited to Avandaryl; gliclazide, glipizide such as but not limited to Glucotrol; glyburide such as but not limited to DiaBeta, Glynase, or Micronase; chlorpropamide such as but not limited to Diabinese; tolazamide such as but not limited to Tolinase; tolbutamide such as but not limited to Orinase or TolTab; rosiglitazone such as but not limited to Avandia; or pioglitazone such as but not limited to Actos. In some embodiments, the treatment regimen includes administration of one or more pharmaceutical drugs, each administered separately to a subject; behavioral modification such as dietary changes and increased daily exercise; or surgery such as bariatric surgery. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Methods of Treating Metabolic Disorder

Aspects of the disclosure are also drawn towards methods of treating a subject afflicted with a metabolic disorder comprising administering to the subject a therapeutically effective amount of a composition described herein.

The term “treating” can refer to partially or completely alleviating, ameliorating, improving, relieving, delaying onset of, inhibiting progression of, reducing severity of, and/or reducing incidence of one or more symptoms, features, or clinical manifestations of a disease, disorder, and/or condition, such as a metabolic disorder. Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition.

The term “subject” or “patient” can refer to any organism to which aspects of the invention can be administered, e.g., for experimental, diagnostic, prophylactic, and/or therapeutic purposes. Subjects to which compounds described herein can be administered can be mammals, for example primates, (such as humans). For veterinary applications, a wide variety of subjects will be suitable, e.g., livestock such as cattle, sheep, goats, cows, swine, and the like; poultry such as chickens, ducks, geese, turkeys, and the like; and domesticated animals for example, pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted herein or another organism that is alive. The term “living subject” refers to the entire subject or organism and not just a part excised (e.g., a liver or other organ) from the living subject.

The term “metabolic disorder” can refer to any disorder that involves an alteration in the normal metabolism of carbohydrates, lipids, proteins, nucleic acids, or a combination thereof. A metabolic disorder can be associated with a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., type 1 diabetes, type 2 diabetes, gestational diabetes), hyperglycemia, hyperinsulinemia, insulin resistance, metabolic syndrome, and obesity.

The term “metabolic syndrome” can refer to a cluster of metabolic abnormalities including abdominal obesity, insulin resistance, glucose intolerance, diabetes, hypertension and dyslipidemia. These abnormalities are known to be associated with an increased risk of vascular events.

The term “obesity” can refer to a condition in which there is an excess of body fat. The operational definition of obesity is based on the Body Mass Index (BMI), which is calculated as body weight per height in meters squared (kg/m²). “Obesity” can refer to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m². As used herein, the term “obesity” is meant to encompass the definitions of obesity herein.

Obesity-induced or obesity-related co-morbidities include, but are not limited to, diabetes, non-insulin dependent diabetes mellitus—type 2, diabetes associated with obesity, impaired glucose tolerance, impaired fasting glucose, insulin resistance syndrome, dyslipidemia, hypertension, hypertension associated with obesity, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. Co-morbidities can include without limitation: hypertension, hyperlipidemia, dyslipidemia, glucose intolerance, cardiovascular disease, sleep apnea, diabetes mellitus, and other obesity-related conditions.

Treatment of obesity and obesity-related disorders can refer to the administration of the compounds or combinations described herein to reduce or maintain the body weight of an obese subject. One outcome of treatment can be reducing the body weight of an obese subject relative to that subject's body weight immediately before the administration of the compounds or combinations described herein. Another outcome of treatment can be preventing body weight regain of body weight previously lost as a result of diet, exercise, or pharmacotherapy. Another outcome of treatment can be decreasing the occurrence of and/or the severity of obesity-related diseases. The treatment can suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment can also result in an alteration of metabolic rate, such as an increase in metabolic rate, rather than or in addition to an inhibition of the reduction of metabolic rate; and/or in minimization of the metabolic resistance that normally results from weight loss.

Prevention of obesity and obesity-related disorders can refer to the administration of the compounds or combinations described herein to reduce or maintain the body weight of a subject at risk of obesity. One outcome of prevention can be reducing the body weight of a subject at risk of obesity relative to that subject's body weight immediately before the administration of the compounds or combinations described herein. Another outcome of prevention can be preventing body weight regain of body weight previously lost as a result of diet, exercise, or pharmacotherapy. Another outcome of prevention can be preventing obesity from occurring if the treatment is administered prior to the onset of obesity in a subject at risk of obesity. Another outcome of prevention can be decreasing the occurrence and/or severity of obesity-related disorders if the treatment is administered prior to the onset of obesity in a subject at risk of obesity. Moreover, if treatment is commenced in already obese subjects, such treatment can prevent the occurrence, progression or severity of obesity-related disorders, such as, but not limited to, arteriosclerosis, Type 2 diabetes, polycystic ovary disease, cardiovascular diseases, osteoarthritis, dermatological disorders, hypertension, insulin resistance, hypercholesterolemia, hypertriglyceridemia, and cholelithiasis.

The term “diabetes,” as used herein, includes both insulin-dependent diabetes mellitus (i.e., IDDM, also known as type 1 diabetes) and non-insulin-dependent diabetes mellitus (i.e., NIDDM, also known as Type 2 diabetes). Type 1 diabetes, or insulin-dependent diabetes, is the result of an absolute deficiency of insulin, the hormone which regulates glucose utilization. Type 2 diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type 2 diabetics are also obese. The compositions of the disclosure are useful for treating both Type 1 and Type 2 diabetes. The compositions are especially effective for treating Type 2 diabetes. The compositions described herein are also useful for treating and/or preventing gestational diabetes mellitus.

Treatment of diabetes mellitus can refer to the administration of a compound or combination of the invention to treat diabetes. One outcome of treatment can be decreasing the glucose level in a subject with elevated glucose levels. Another outcome of treatment can be decreasing insulin levels in a subject with elevated insulin levels. Another outcome of treatment is decreasing plasma triglycerides in a subject with elevated plasma triglycerides. Another outcome of treatment is decreasing LDL cholesterol in a subject with high LDL cholesterol levels. Another outcome of treatment is increasing HDL cholesterol in a subject with low HDL cholesterol levels. Another outcome of treatment is increasing insulin sensitivity. Another outcome of treatment can be enhancing glucose tolerance in a subject with glucose intolerance. Yet another outcome of treatment can be decreasing insulin resistance in a subject with increased insulin resistance or elevated levels of insulin.

Prevention of diabetes mellitus refers to the administration of a compound or combination described herein to prevent the onset of diabetes in a subject in need thereof.

According to the invention, the term “NASH” or “Non-Alcoholic SteatoHepatitis” refers to a Non-Alcoholic Fatty Liver Disease condition characterized by the concomitant presence of liver steatosis, hepatocyte ballooning and liver inflammation at histological examination, (i.e. NAS>3, with at least 1 point in steatosis, at least 1 point in lobular inflammation and at least 1 point in the hepatocyte ballooning scores) in the absence of excessive alcohol consumption and after excluding other liver diseases like viral hepatitis (HCV, HBV). Embodiments can prevent the progression of NASH, which includes, for example, spider hemangioma, ascites, splenomegaly, hardening of the liver's edge, palm erythema, flapping tremor, liver fibrosis, one or more symptoms of degeneration and hepatocellular carcinoma. Increased nonalcoholic steatohepatitis is also associated with symptoms such as cirrhosis and liver failure, and is associated with liver transplantation.

The term “administration” can refer to introducing a composition or substance, such as a composition comprising naringenin and at least one carotenoid, into a subject. Non-limiting examples of modes of administration are described elsewhere in this disclosure. Any route of administration can be utilized including, for example, parenteral, oral, or transdermal, or any combination thereof.

Methods of Fat Reduction

Aspects of the disclosure are drawn towards methods of weight and fat reduction. For example, “weight and body fat reduction” can refer to the presence of a reduced amount of weight or body fat after administration of a therapeutically effective amount of compounds or compositions described herein. The phrase “reduce body fat” or “reduction of body fat”, for example, can refer to a decrease in the amount of weight in an individual attributable to fat cells.

This can be measured by many known methods, such as Body Mass Index, with skin fold calipers, by DEXA (Dual Energy X-ray Absorptiometry) and/or by hydrostatic weighing. The methods of the invention described herein can reduce body fat by about 5%, by about 10%, or by about 20% or more of the total weight of the individual. For example, this translates into a weight loss of about 2 to 3 pounds per week for an individual. In one embodiment, the amount of weight loss can be about 1% body fat/week. Non-limiting examples of the body fat (adipose tissue) include visceral fat, perirenal fat, mesenteric fat, epididymal fat, and subcutaneous fat.

For example, studies using a 150 mg three times a day of naringenin alone show the loss of 2.3% of body weight in 8 weeks, which can be 4.6% at the 6 month plateau. Without wishing to be bound by theory, a dose of 12 mg/d of beta carotene and 300 mg of naringenin three times a day will result in an average weight loss at the six month plateau of between 5% to 10% body weight loss. Weight loss is 75% fat and 20% to 25% lean tissue but this can vary based on the treatment regimen and/or subject variability. There is a great variability in the response to any weight loss intervention. Thus, there can be some subjects that lose 20% or more.

Aspects of the disclosure are also drawn towards methods of converting white fat to brown fat in a subject. For example, the conversion of white fat to brown fat can be indicated by an increase in metabolic rate, weight loss and improvement in insulin resistance. Also, adiponectin can be measured as an indicator of insulin resistance.

White fat comprises large, white cells that are stored under the skin or around the organs in the belly, arms, buttocks, and thighs. These fat cells are the body's way of storing energy for later use. White fat also plays a large role in the function of hormones such as estrogen, leptin (one of the hormones that stimulates hunger), insulin, cortisol (a stress hormone), and growth hormone. While some white fat is necessary for good health, too much white fat is very harmful. Healthy body fat percentages range depending on your level of fitness or physical activity. Body fat percentage higher than recommended can put you at risk for the health issues, such as type 2 diabetes, coronary artery disease, high blood pressure, stroke, hormone imbalances, pregnancy complications, kidney disease, liver disease, and cancer.

Brown fat is a type of fat primarily found in babies, although adults do still retain a very small amount of brown fat, for example in the neck and shoulders. Brown fat burns fatty acids to keep a subject warm.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Synergy of Beta-Carotene and Naringenin to Increase UCP-1.

Naringenin at 10 micromolar has been demonstrated in our lab to brown white human adipose cells in culture by increasing RNA and protein expression of uncoupling protein-1 (documented an increase in oxygen consumption by Seahorse), adipose tissue glyceride lipase and carnitine palmitoyl transferase-1 (associated with increased lipolysis), and glucose transporter-4 and carbohydrate response element binding protein alpha and beta (increased glucose utilization). Adiponectin RNA expression and adiponectin protein levels (as shown by Western blot analysis) also went up (improves insulin sensitivity). Beta carotene at 1 micromolar has been demonstrated in our lab to increase RNA expression in human adipocytes of uncoupling protein-1 (oxygen consumption), adipose tissue glyceride lipase (lipolysis) and adiponectin (insulin sensitivity). The combination of naringenin at 10 micromolar and beta carotene at 1 micromolar increased UCP-1 and GLUT4 to a significantly greater degree that the combined individual contributions of naringenin and beta carotene alone showing that the combination is synergistic in browning white fat (p<0.01). The browning of white fat can be an effective treatment for obesity and type 2 diabetes, which are major public health problems.

Embodiments herein comprise a treatment for obesity, insulin resistance, type 2 diabetes, metabolic syndrome, and non-alcoholic steohepatitis. The invention can be applied as a topical cream for local fat reduction, however other routes of administration known to the skilled artisan can be utilized as well. For example, the combination of naringenin and beta carotene is food and can be developed as a medical food or a dietary herbal supplement. For example, clinical trials can demonstrate efficacy, but will not require the pre-clinical safety studies associated with a new chemical entity used in drug development.

Naringenin and beta carotene are safe foods and can offer a treatment for childhood obesity, which does not have an effective treatment, in addition to treating adult obesity. Since the development costs will be much less than what can be required for a new drug, the product can be sold at a profit for a much lower price. This can be a big market advantage, since the approved obesity drugs for long-term use are not very successful in the market due to their high prices. The approved medications for the treatment of obesity reduce food intake. In embodiments, the invention can be taken in combination with approved drugs for obesity that reduce food intake only, to safely give greater weight loss and improve insulin sensitivity. Naringenin and beta carotene increase metabolic rate and offer a new approach to the treatment of obesity, and one that obese patients feel is at the root of their obesity problem.

### Example 2

Synergy between Naringenin and β-carotene for increasing Energy Expenditure and Insulin Sensitivity, and Improving Fatty Liver Disease

Naringenin is a citrus flavonoid that acts on the liver to reduce cholesterol, triglycerides and insulin resistance. A growing number of studies in obese rodent models have shown that dietary naringenin increases levels of transcriptional regulators of hepatic fat oxidation including PPARγ, PPARα, and Pgc-1α and downstream target genes for fat oxidation [1-6]. Naringenin decreases activity and levels of sterol regulatory element binding protein 1/2 (SREBP1/2), lipogenic enzymes and cholesterol synthesis in multiple high fat diet and high cholesterol models. In addition, naringenin reduces serum and hepatic levels of triglycerides and cholesterol [7-10]. Direct activation of PPARγ and PPARα by naringenin has been demonstrated using reporter gene assays [11].

The effects of naringenin on adipose tissue in obese rodents have been studied. Naringenin treatment reduces adipocyte hypertrophy, macrophage invasion, inflammation and production of inflammatory cytokines [3, 12-14].

Long-term feeding of naringenin has potent effects in ovariectomized mice, a model for metabolic alterations in post-menopausal women. Naringenin prevented the weight gain caused by ovariectomy and improved fasting glucose, insulin and cholesterol levels [14]. In a later study, ovariectomized mice were fed a high fat diet until body weight was almost twice the baseline level. Dietary naringenin reversed the weight gain and improved glucose metabolism and muscle function [15].

The beneficial effects of naringenin on blood glucose and insulin sensitivity have been shown in obese human subjects administered grapefruit juice three times a day. After 12 weeks, there was a significant reduction in insulin and blood sugar that was associated with weight loss [16]. Research examining the mechanism underlying reduction in blood glucose by naringenin has demonstrated a role for activation of PI3K, IRS1, PPARγ and inhibition of PEPCK in liver and liver cell lines [7, 8, 17, 18]. Enhanced glucose uptake with activation of AMPK after Naringenin treatment was observed in muscle cells [19].

Flavonoids such as naringenin are biotransformed into their metabolites by gut microbiota; however, flavonoids also modulate the composition of the gut microbial community. The formation of citrus flavonoids and the modulation of gut microbiota can both contribute to the health benefits they confer[20]. Examination of fecal metabolomic profiles indicate that naringenin is depleted in mice fed a high fat diet as a result of a stimulation of microbiome mediated flavonoid degrading capacity. In these mice, the addition of naringenin to the high fat diet attenuated weight gain by induction of the major thermogenic factor uncoupling protein 1 (UCP1) in brown adipose tissue [21].

Flavanones have been shown to selectively inhibit 11-beta hydroxysteroid dehydrogenase type 1 (11-beta HSD-1), an enzyme in fat tissue that converts the inactive precursor of cortisone into active cortisol, and naringenin is a flavanone [22]. Inhibitors of 11-beta HSD-1 have the potential to treat obesity and the metabolic syndrome [23, 24]. Cortisol causes thinning of the skin, atrophy, impaired wound healing and 11-beta HSD-1 is increased in ageing human skin. Studies have shown that topical treatment to the skin with inhibitors of 11-beta HSD-1 can accelerate wound healing and improve age-associated impairments in dermal integrity [25].

In pharmacokinetic studies in humans, a 10 μM concentration of naringenin has been shown to be physiologically attainable [26]. In our preclinical lab at PBRC, human subcutaneous adipocyte cell cultures were treated with naringenin at a concentration of 10 μM for seven days. Significant increases in genes that regulate thermogenesis and insulin sensitivity were observed, including the brown adipocyte markers Uncoupling Protein 1(UCP1), Glucose transporter type 4 (GLUT4) and carnitine palmitoyltransferase 1 (CPT-1) in human subcutaneous adipocyte cultures.

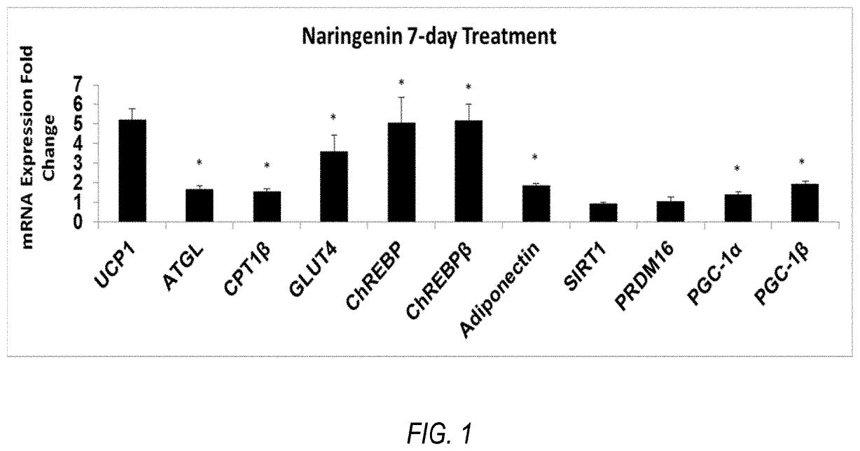

Referring to FIG. 1, Naringenin induces expression of genes for energy expenditure and glucose utilization.

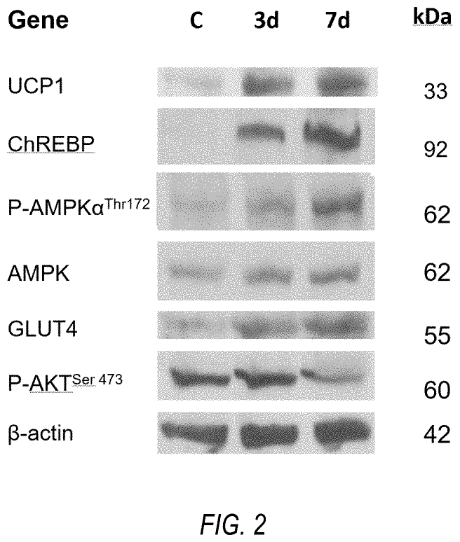

Differentiated human adipocytes were treated with 10 μM naringenin for 7 days. Gene expression was analyzed using Taqman RT-PCR and values are expressed as fold increase over untreated controls (n=5). Corresponding increases in the protein levels of UCP1, GLUT4, and ChREBP were observed. Importantly, naringenin activates AMPK, as indicated by an increase in phosphor-AMPK, the activated form (see FIG. 2). Activated AMPK induces glucose consumption and fat oxidation in adipose tissue [27].

Referring to FIG. 2, human adipocyte cultures were treated with naringenin for 0, 3 or 7d. Total protein was isolated and analyzed by Western Blotting.

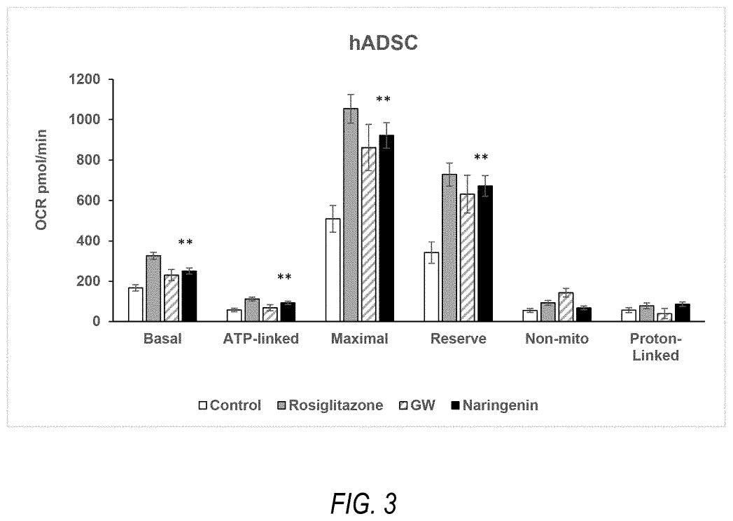

These data indicate that Naringenin induces markers of energy expenditure and adipokines that improve whole body insulin sensitivity in human white fat cells. Our studies of oxygen consumption rate in naringenin-treated adipocytes using a Seahorse XF24 analyzer show that naringenin also significantly increases basal and maximal energy expenditure in human fat cells (FIG. 3). Thus, not only does naringenin increase UCP-1 mRNA and protein levels, but it also increases energy expenditure, confirming that these increases in RNA and protein have a functional correlate.

Referring to FIG. 3, oxygen consumption rate in human adipocytes after Naringenin treatment. Cells were treated for 7d and OCR was measured using a Seahorse XF24. Rosiglitizone and GW7647 (activators of PPARγ and PPARα) were included as positive controls.

Beta-carotene (BC) is the main source of vitamin A in the human diet. BC is converted to retinal by the cytosolic enzyme β-carotene-15,15′-oxygenase (BCO1), which is expressed in mammalian cells. Retinal can then be metabolized to biologically active derivatives, such as by irreversible oxidation to retinoic acid or by reduction to retinol [28]. Retinoic acid isomers are the active BC derivatives that regulate expression of genes involved in energy expenditure and lipid metabolism. All-trans retinoic acid (atRA) can bind any of the three isoforms of retinoic acid receptors (RARs) and 9-cis retinoic acid binds RARs and three retinoic X receptor isoforms (RXRs). When bound to ligand, RAR transcription factors form heterodimers with RXRs and positively regulate genes for uncoupling proteins and lipases by binding RAR response elements in the promoter regions of these target genes. The RXRs are nuclear receptor coactivators for PPARγ and thyroid hormone receptor, among a number of others [29]. Both liver and adipose tissue are major targets of the actions of dietary BC on lipid metabolism.

Adipose tissue is a major storage site for BC and its derivatives, and there is a high correlation between plasma levels and abdominal adipose tissue levels for both men and women. In adipocytes, carotenoids can be found in lipid droplets and cell membranes [30]. Dietary BC supplementation of 150 mg/kg/day in mice reduced adiposity without altering food intake or bodyweight. The effect was abolished in BCO1 knockout mice, indicating that the anti-adiposity effect of BC occurs through its retinoid derivatives [31]. A similar study showed that reduction of lipid storage by BC in adipocytes requires conversion of BC to retinoid derivatives by BCO1 and the mechanism involves repression of adipogenic PPAR γ target genes through RAR activation [32].

At the cellular level, retinoic acid metabolites of BC lower adiposity through a dual mechanism in adipose tissue[33]. In preadipocytes, retinoic acid inhibits the adipogenic program by suppressing expression of Zfp423, a key adipogenic transcription factor that also inhibits thermogenesis [34, 35]. In mature adipocytes, it induces a program of thermogenic gene expression and mitochondrial fat oxidation. Treatment of a murine adipocyte cell line, 3T3-L1, with atRA induced Ucp1, Pgc1β, mitochondrial genes and oxygen consumption [36]. Evidence is accumulating that atRA induces UCP1, PPARα and markers of fat oxidation in white adipocytes through the activation of RAR and PPAR coactivators [37].

Beta-carotene metabolites also stimulate lipid metabolism in liver. In mice injected daily with 100 mg/kg of atRA, a reduction in body weight and adiposity were observed with reductions in liver TG and glycogen content[38]. Retinoic acid induces CPT-1 and lipid oxidation in HEPG2 cells, a human liver-derived cell line[39]. Vitamin A plays a role in liver lipid metabolism and reduced levels in serum have been correlated to fatty liver diseases [40]. Furthermore, retinoids regulate hepatic expression of key apolipoproteins in HDL, a serum protein complex which is linked to protection against coronary artery disease [41, 42].

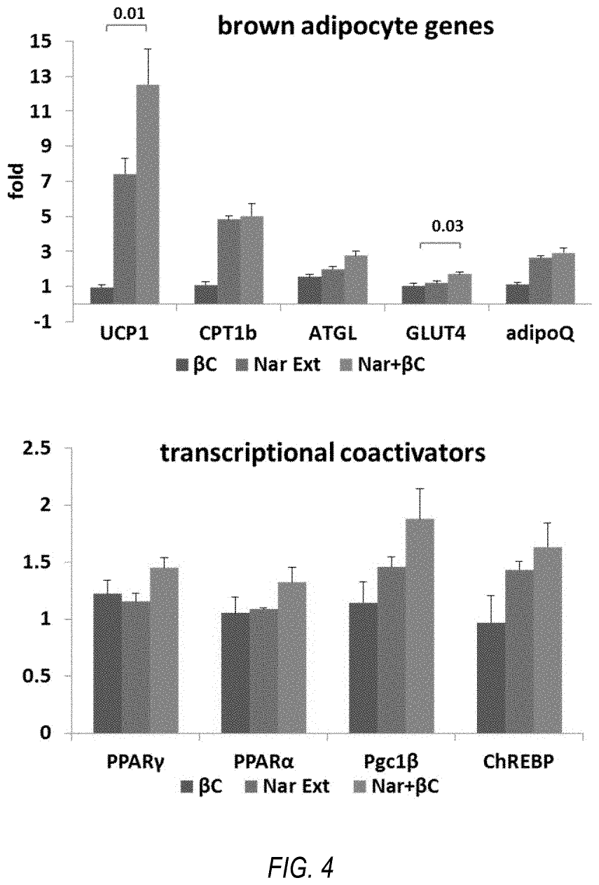

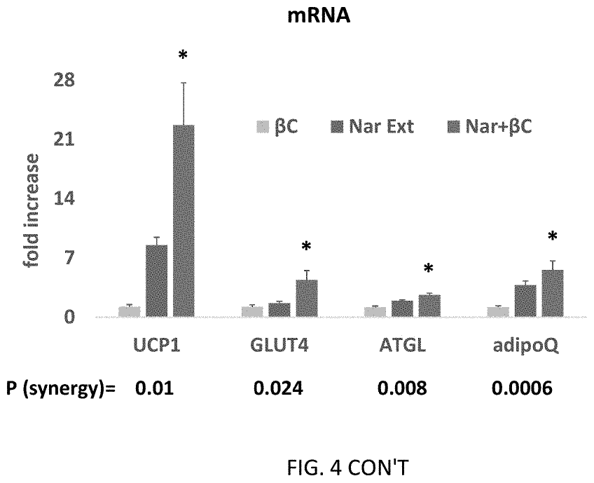

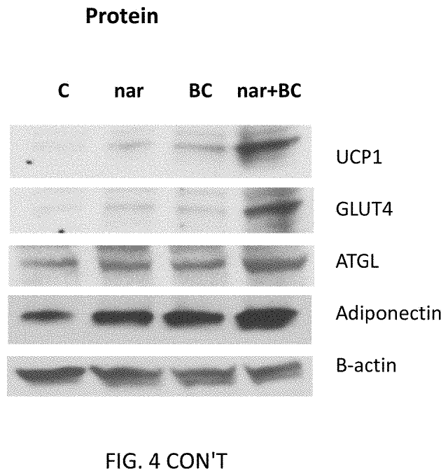

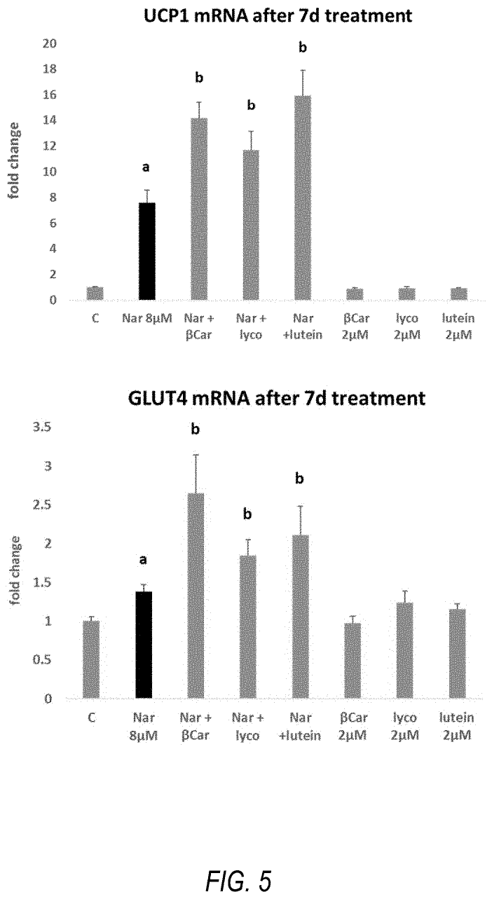

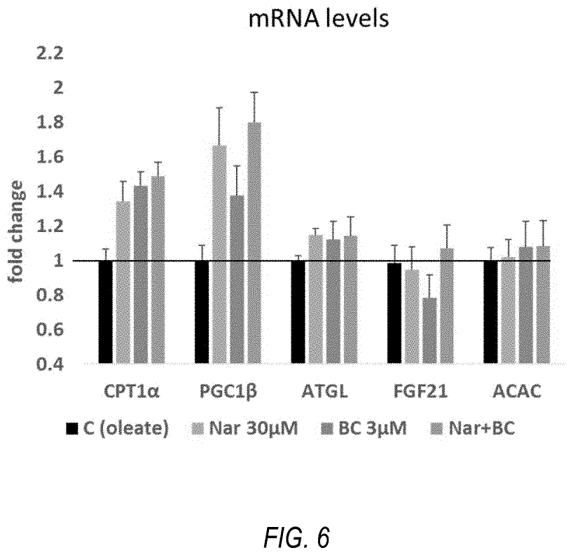

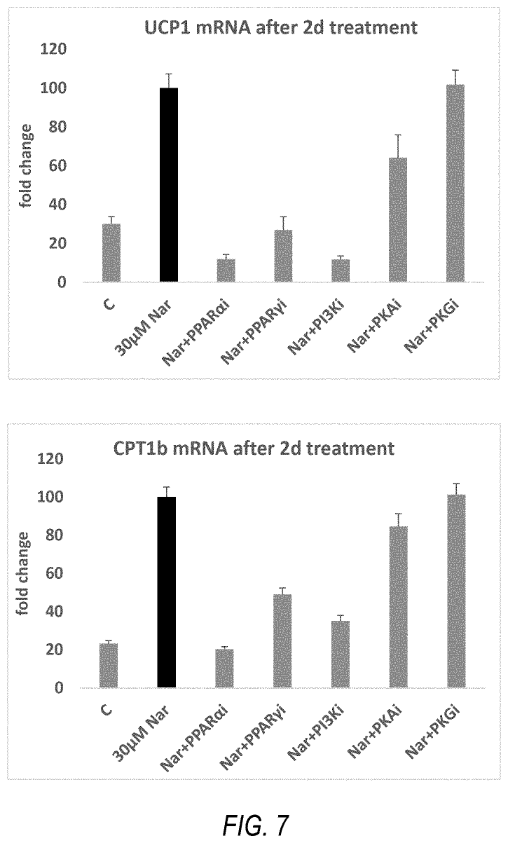

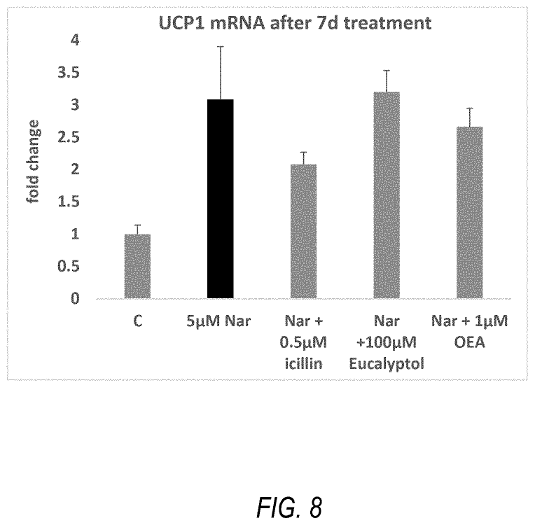

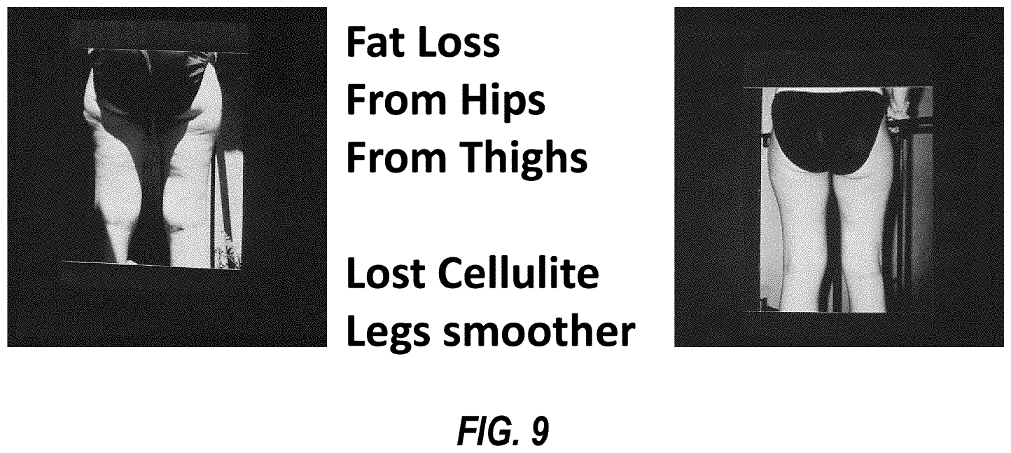

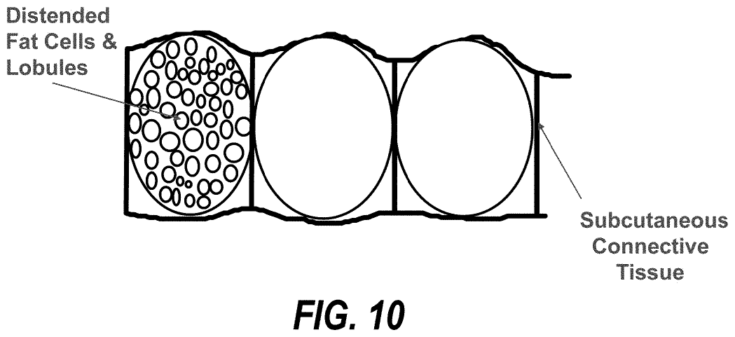

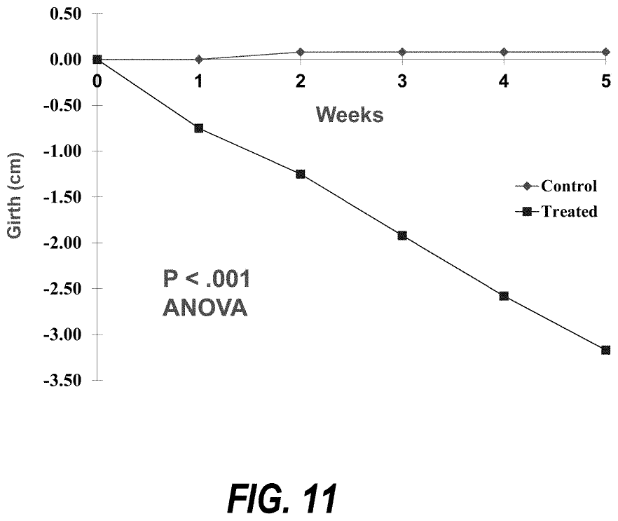

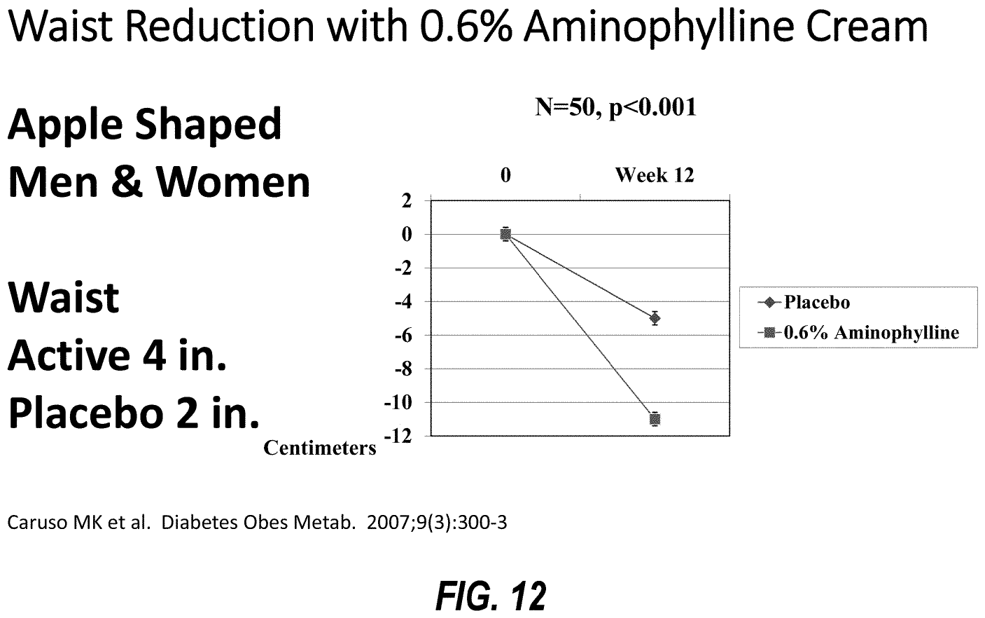

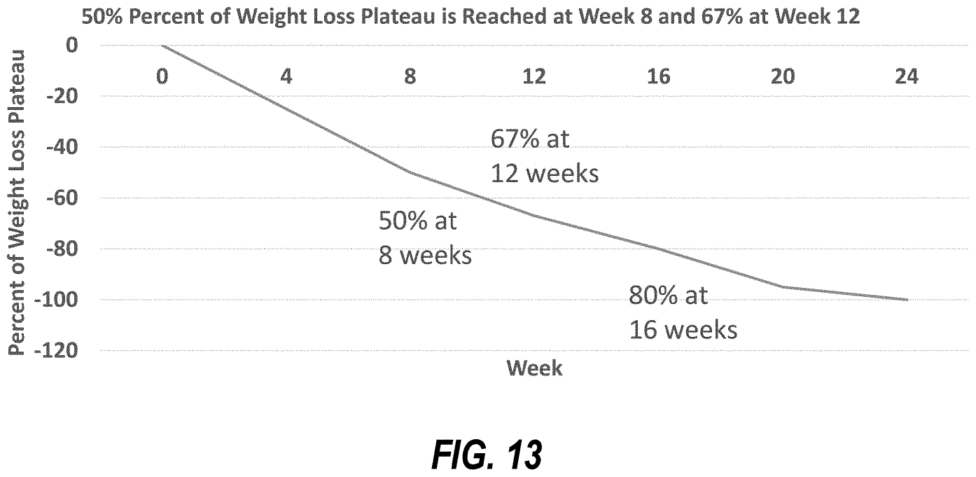

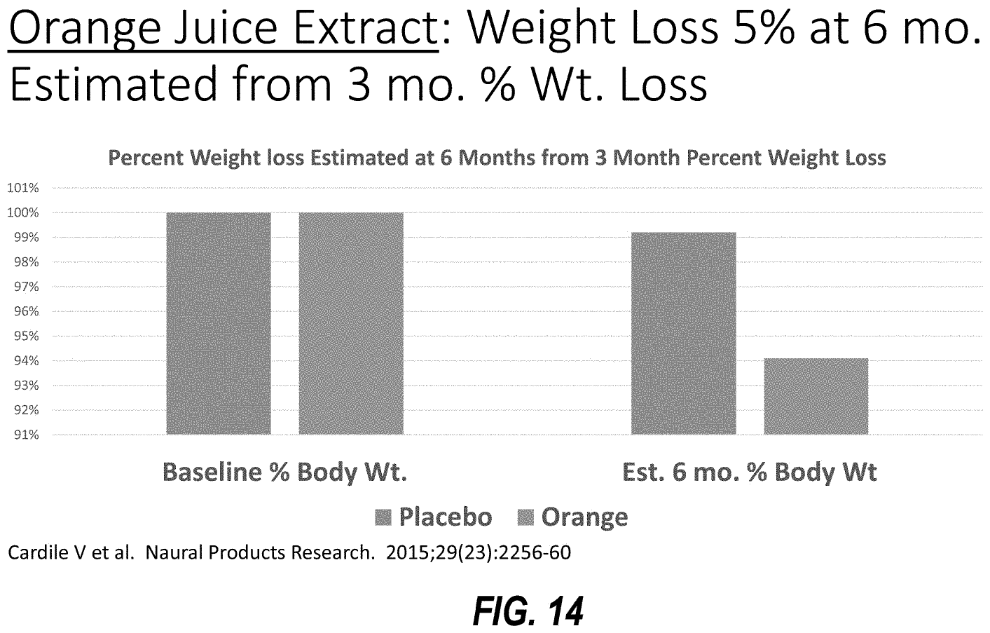

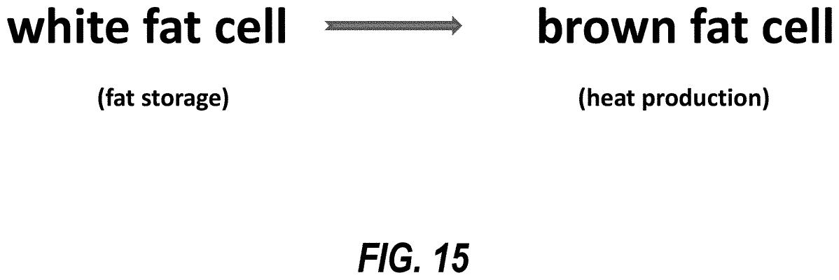

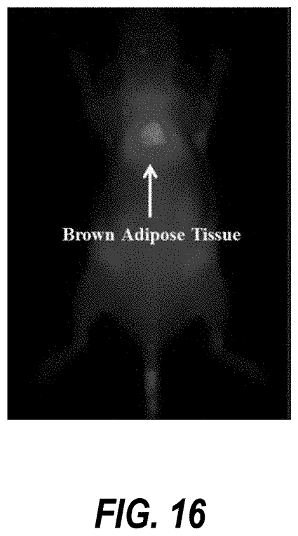

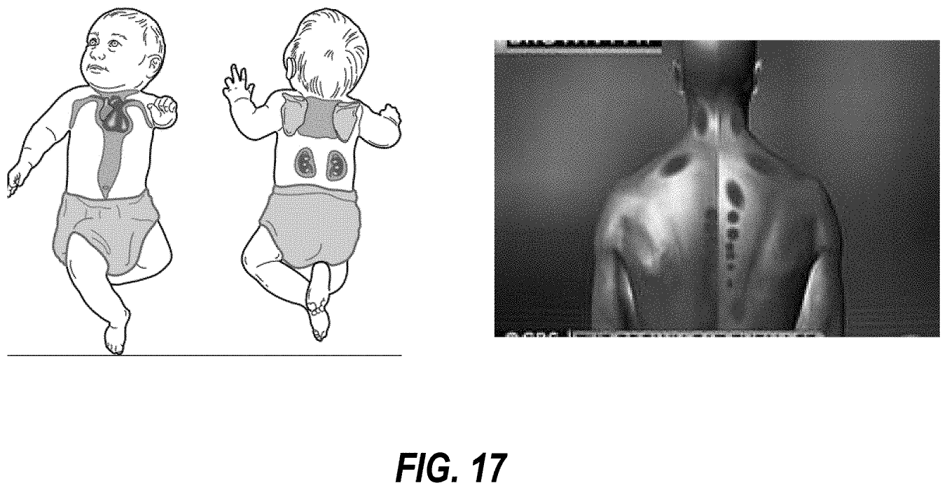

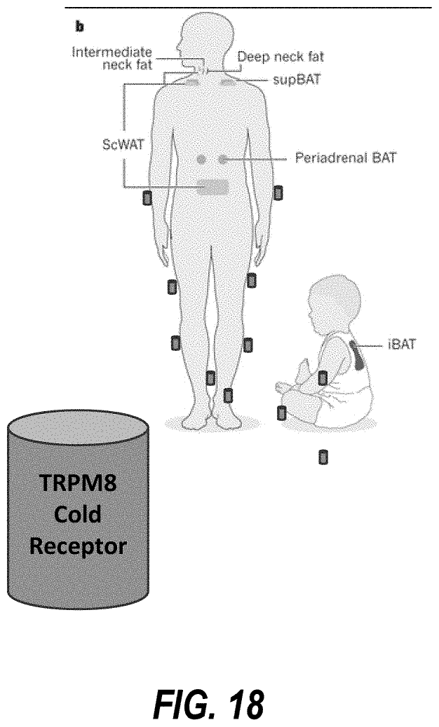

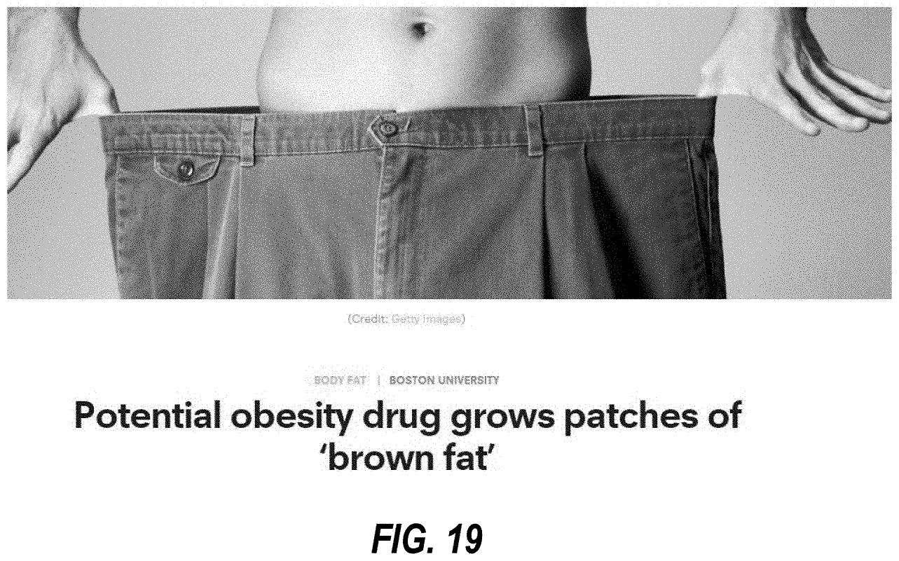

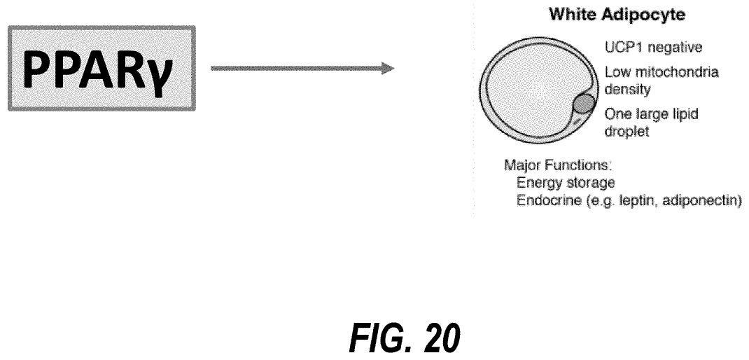

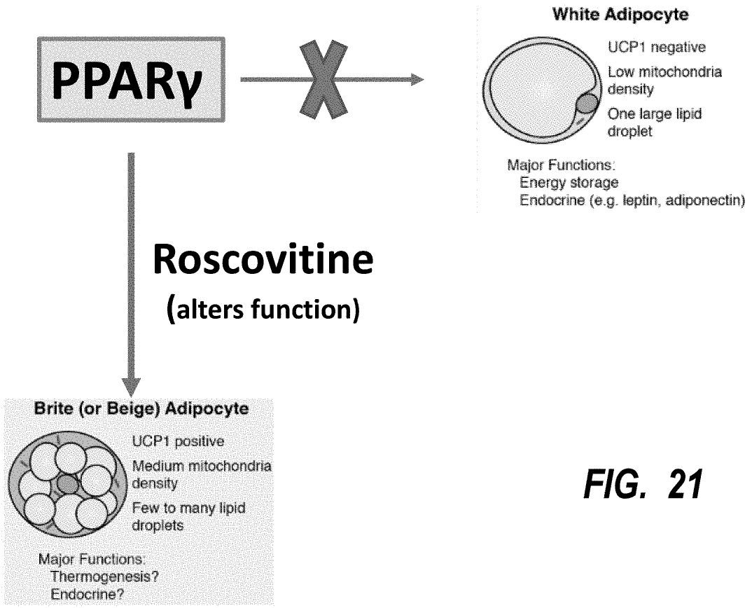

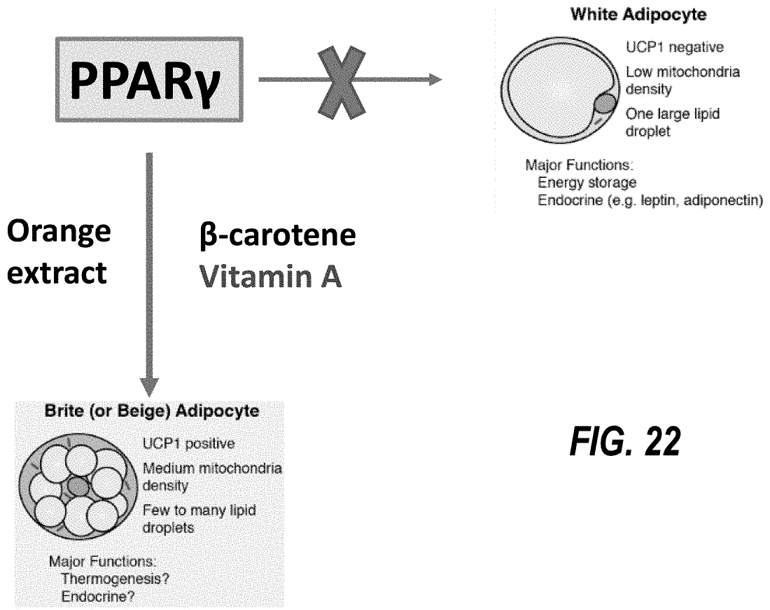

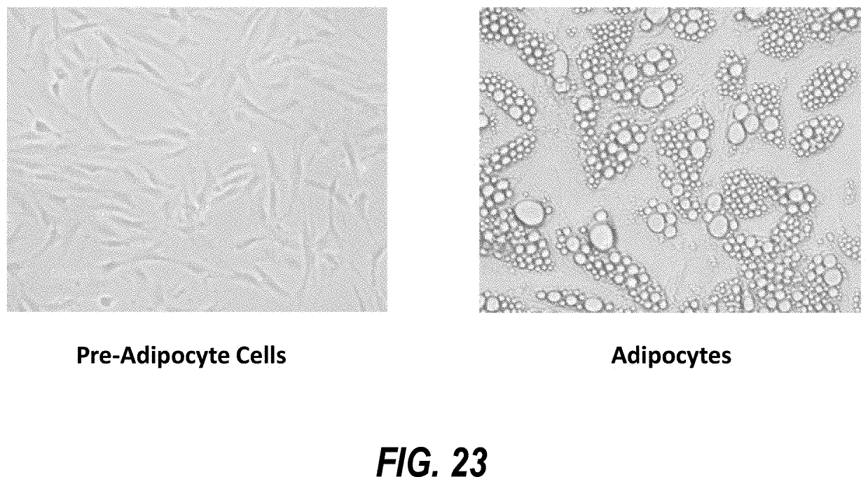

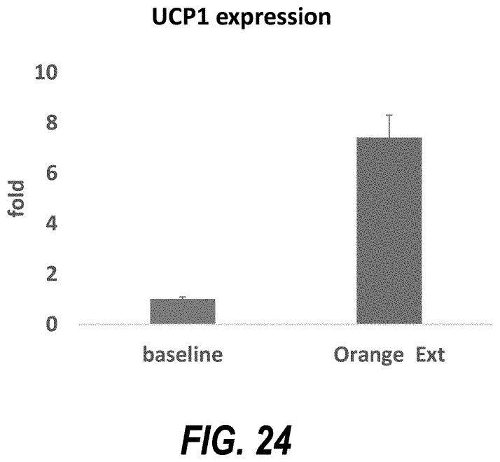

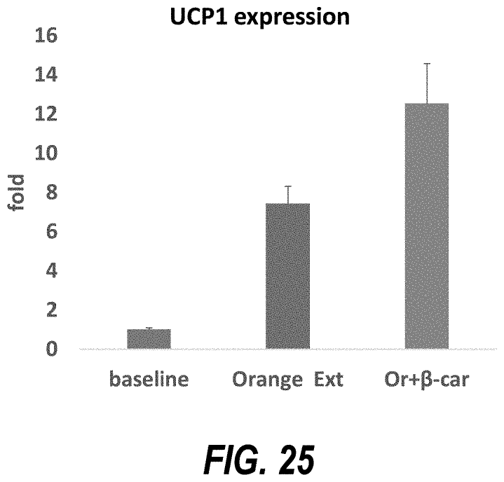

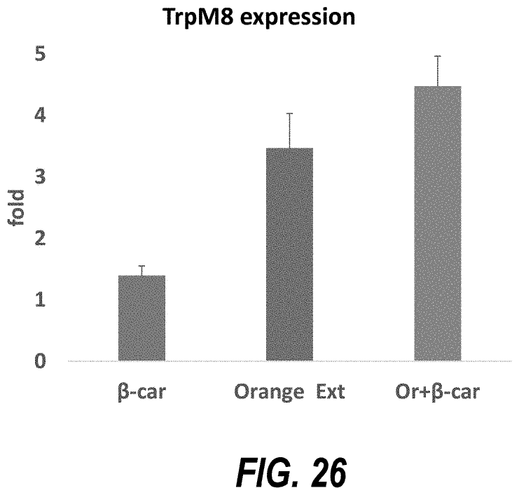

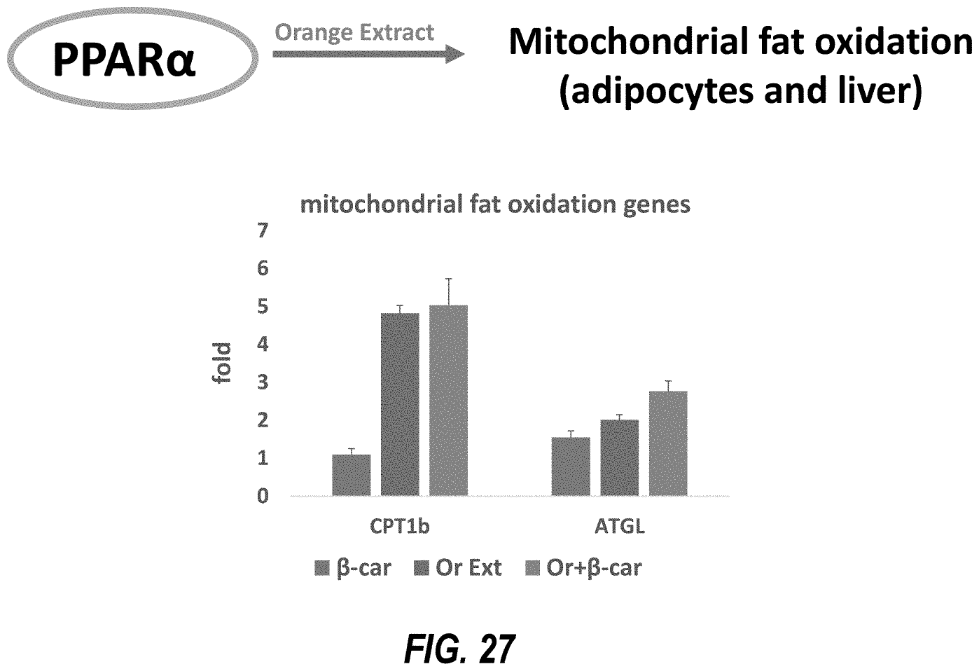

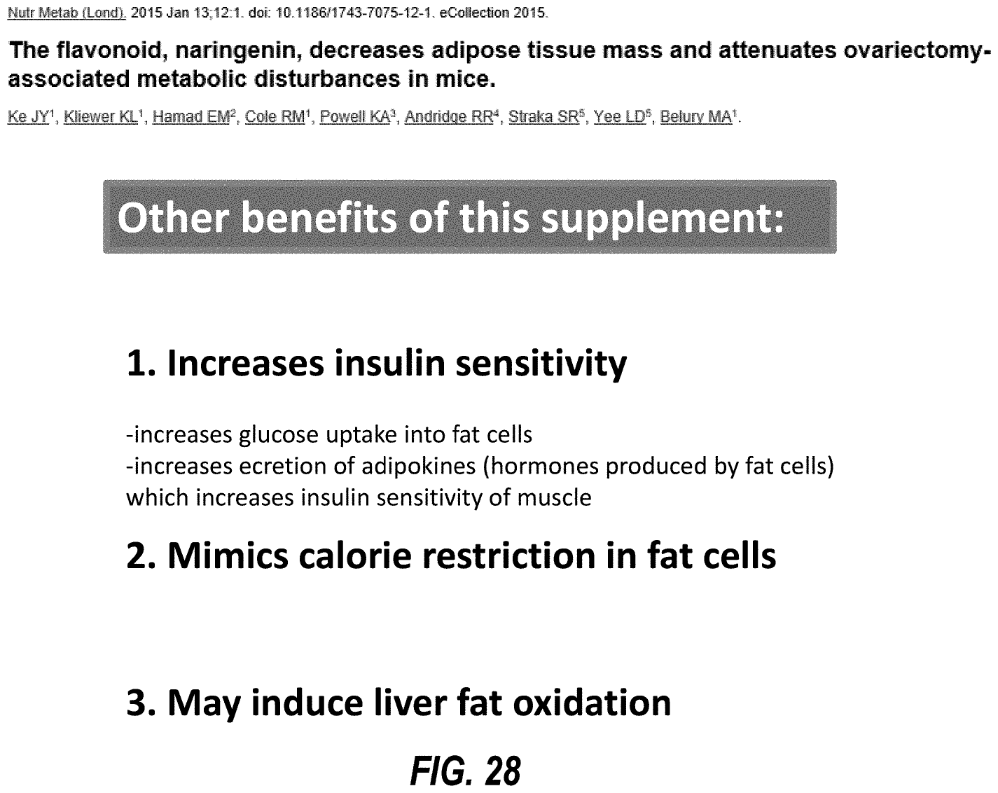

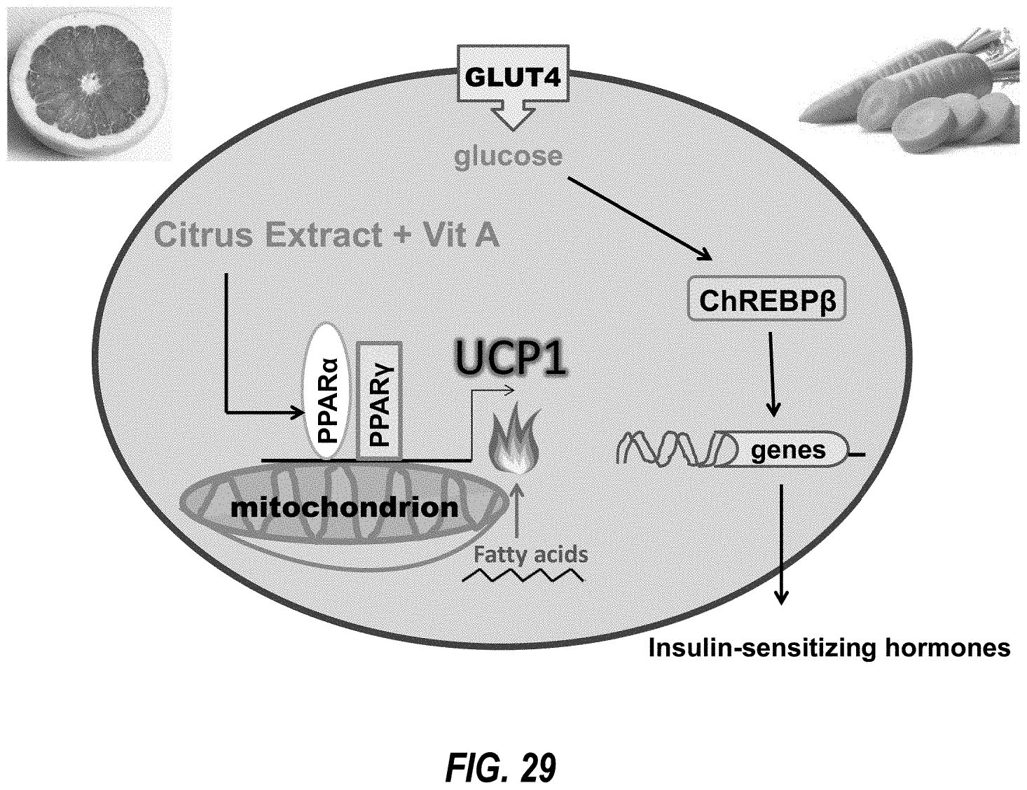

Our laboratory tested a combination of naringenin, a natural dual PPARα/PPARγ activator and BC, an activator of RARs and RXRs through retinoid metabolites, to synergistically enhance fat oxidation and thermogenic energy expenditure genes in human adipocytes. The results showed significant synergy for the elevation of UCP1 and GLUT4 mRNAs in cells exposed to the combination (FIG. 4). The naringenin plus BC combination also induced higher levels of ATGL, adiponectin, PPARα and PPARγ in comparison to single treatment.

Referring to FIG. 4, Naringenin and BC treatment of human subcutaneous adipocytes from obese donors. These results indicate that a supplement composed of naringenin extract and β-carotene has the potential to treat obesity, insulin resistance and fatty liver diseases.

Clinical data and relevance: There have been no clinical trials to investigate the effects of naringenin. We are currently conducting a single dose escalation study to determine the pharmacokinetics of naringenin at the Pennington Biomedical Research Center. For BC, two double-blind, placebo controlled clinical trials have been conducted on obese children using doses of 4 mg/day. In subjects given BC, there was a reduction in BMI z-score, subcutaneous adipose tissue, visceral adipose tissue, waist circumference, increases in circulating adiponectin and BC, and improved insulin sensitivity after six months [43, 44]. Without wishing to be bound by theory, the combination therapy will work in synergy to activate target genes for fat oxidation in both adipose tissue and liver, given the mechanism of naringenin to stimulate PPAR activity and the activation of PPAR transcriptional coactivators by BC metabolites. In addition, the adiponectin promoter has a PPAR response element (PPRE) and higher circulating adiponectin levels can improve insulin sensitivity by acting on multiple target tissues. Without wishing to be bound by theory, the combination will have a stronger effect on weight loss than BC alone.

References Cited in this Example:
Mulvihill, E. E., et al., Naringenin prevents dyslipidemia, apolipoprotein B overproduction, and hyperinsulinemia in LDL receptor-null mice with diet-induced insulin resistance. Diabetes, 2009. 58(10): p. 2198-210.
2. Assini, J. M., et al., Naringenin prevents obesity, hepatic steatosis, and glucose intolerance in male mice independent of fibroblast growth factor 21. Endocrinology, 2015. 156(6): p. 2087-102.
3. Assini, J. M., et al., Naringenin prevents cholesterol-induced systemic inflammation, metabolic dysregulation, and atherosclerosis in Ldlr(−)/(−) mice. J Lipid Res, 2013. 54(3): p. 711-24.
4. Christensen, K. B., et al., Identification of bioactive compounds from flowers of black elder (Sambucus nigra L.) that activate the human peroxisome proliferator-activated receptor (PPAR) gamma. Phytother Res, 2010. 24 Suppl 2: p. S129-32.
5. Cho, K. W., et al., Dietary naringenin increases hepatic peroxisome proliferators-activated receptor alpha protein expression and decreases plasma triglyceride and adiposity in rats. Eur J Nutr, 2011. 50(2): p. 81-8.
6. Huong, D. T., Y. Takahashi, and T. Ide, Activity and mRNA levels of enzymes involved in hepatic fatty acid oxidation in mice fed citrus flavonoids. Nutrition, 2006. 22(5): p. 546-52.
7. Sharma, A. K., et al., Up-regulation of PPARgamma, heat shock protein-27 and -72 by naringin attenuates insulin resistance, beta-cell dysfunction, hepatic steatosis and kidney damage in a rat model of type 2 diabetes. Br J Nut, 2011. 106(11): p. 1713-23.
8. Borradaile, N. M., L. E. de Dreu, and M. W. Huff, Inhibition of net HepG2 cell apolipoprotein B secretion by the citrus flavonoid naringenin involves activation of phosphatidylinositol 3-kinase, independent of insulin receptor substrate-1 phosphorylation. Diabetes, 2003. 52(10): p. 2554-61.
9. Kim, H. J., et al., Naringin alters the cholesterol biosynthesis and antioxidant enzyme activities in LDL receptor-knockout mice under cholesterol fed condition. Life Sci, 2004. 74(13): p. 1621-34.
10. Lee, S. H., et al., Cholesterol-lowering activity of naringenin via inhibition of 3-hydroxy-3-methylglutaryl coenzyme A reductase and acyl coenzyme A: cholesterol acyltransferase in rats. Ann Nutr Metab, 1999. 43(3): p. 173-80.
11. Goldwasser, J., et al., Transcriptional regulation of human and rat hepatic lipid metabolism by the grapefruit flavonoid naringenin: role of PPARalpha, PPARgamma and LXRalpha. PLoS One, 2010. 5(8): p. e12399.
12. Yoshida, H., et al., Naringenin suppresses macrophage infiltration into adipose tissue in an early phase of high-fat diet-induced obesity. Biochem Biophys Res Commun, 2014. 454(1): p. 95-101.
13. Yoshida, H., et al., Citrus flavonoid naringenin inhibits TLR2 expression in adipocytes. J Nutr Biochem, 2013. 24(7): p. 1276-84.
14. Ke, J. Y., et al., The flavonoid, naringenin, decreases adipose tissue mass and attenuates ovariectomy-associated metabolic disturbances in mice. Nutr Metab (Lond), 2015. 12: p. 1.
15. Ke, J. Y., et al., Citrus flavonoid, naringenin, increases locomotor activity and reduces diacylglycerol accumulation in skeletal muscle of obese ovariectomized mice. Mol Nutr Food Res, 2016. 60(2): p. 313-24.
16. Fujioka, K., et al., The effects of grapefruit on weight and insulin resistance: relationship to the metabolic syndrome. J Med Food, 2006. 9(1): p. 49-54.
17. Kannappan, S. and C. V. Anuradha, Naringenin enhances insulin-stimulated tyrosine phosphorylation and improves the cellular actions of insulin in a dietary model of metabolic syndrome. Eur J Nutr, 2010. 49(2): p. 101-9.
18. Park, H. J., et al., Citrus unshiu peel extract ameliorates hyperglycemia and hepatic steatosis by altering inflammation and hepatic glucose-and lipid-regulating enzymes in db/db mice. J Nutr Biochem, 2013. 24(2): p. 419-27.
19. Zygmunt, K., et al., Naringenin, a citrus flavonoid, increases muscle cell glucose uptake via AMPK. Biochem Biophys Res Commun, 2010. 398(2): p. 178-83.
20. Cassidy, A. and A. M. Minihane, The role of metabolism (and the microbiome) in defining the clinical efficacy of dietary flavonoids. Am J Clin Nutr, 2017. 105(1): p. 10-22.
21. Thaiss, C. A., et al., Persistent microbiome alterations modulate the rate of post-dieting weight regain. Nature, 2016.
22. Lee, Y. S., et al., Grapefruit juice and its flavonoids inhibit 11 beta-hydroxysteroid dehydrogenase. Clin Pharmacol Ther, 1996. 59(1): p. 62-71.
23. Anagnostis, P., et al., 11beta-Hydroxysteroid dehydrogenase type 1 inhibitors: novel agents for the treatment of metabolic syndrome and obesity-related disorders? Metabolism, 2013. 62(1): p. 21-33.
24. Schweizer, R. A., et al., A rapid screening assay for inhibitors of 11beta-hydroxysteroid dehydrogenases (11beta-HSD): flavanone selectively inhibits 11beta-HSD1 reductase activity. Mol Cell Endocrinol, 2003. 212(1-2): p. 41-9.
25. Tiganescu, A., et al., 11beta-Hydroxysteroid dehydrogenase blockade prevents age-induced skin structure and function defects. J Clin Invest, 2013. 123(7): p. 3051-60.
26. Kanaze, F. I., et al., Pharmacokinetics of the citrus flavanone aglycones hesperetin and naringenin after single oral administration in human subjects. Eur J Clin Nutr, 2007. 61(4): p. 472-7.
27. Yamauchi, T., et al., Adiponectin stimulates glucose utilization and fatty-acid oxidation by activating AMP-activated protein kinase. Nature Medicine, 2002. 8: p. 1288.
28. Bonet, M. L., et al., Carotenoids and their conversion products in the control of adipocyte function, adiposity and obesity. Arch Biochem Biophys, 2015. 572: p. 112-125.
29. Bonet, M. L., et al., Carotenoids in Adipose Tissue Biology and Obesity. Subcell Biochem, 2016. 79: p. 377-414.
30. El-Sohemy, A., et al., Individual carotenoid concentrations in adipose tissue and plasma as biomarkers of dietary intake. Am J Clin Nutr, 2002. 76(1): p. 172-9.
31. Amengual, J., et al., Beta-carotene reduces body adiposity of mice via BCMO1. PLoS One, 2011. 6(6): p. e20644.
32. Lobo, G. P., et al., Beta,beta-carotene decreases peroxisome proliferator receptor gamma activity and reduces lipid storage capacity of adipocytes in a beta, beta-carotene oxygenase 1-dependent manner. J Biol Chem, 2010. 285(36): p. 27891-9.
33. Noy, N., The one-two punch: Retinoic acid suppresses obesity both by promoting energy expenditure and by inhibiting adipogenesis. Adipocyte, 2013. 2(3): p. 184-7.
34. Wang, B., et al., Retinoic acid inhibits white adipogenesis by disrupting GADD45A-mediated Zfp4423 DNA demethylation. J Mol Cell Biol, 2017. 9(4): p. 338-349.
35. Shao, M., et al., Zfp4423 Maintains White Adipocyte Identity through Suppression of the Beige Cell Thermogenic Gene Program. Cell Metab, 2016. 23(6): p. 1167-1184.
36. Tourniaire, F., et al., All-trans retinoic acid induces oxidative phosphorylation and mitochondria biogenesis in adipocytes. J Lipid Res, 2015. 56(6): p. 1100-9.
37. Mercader, J., et al., Remodeling of white adipose tissue after retinoic acid administration in mice. Endocrinology, 2006. 147(11): p. 5325-32.
38. Amengual, J., et al., Retinoic acid treatment enhances lipid oxidation and inhibits lipid biosynthesis capacities in the liver of mice. Cell Physiol Biochem, 2010. 25(6): p. 657-66.
39. Amengual, J., et al., Induction of carnitine palmitoyl transferase 1 and fatty acid oxidation by retinoic acid in HepG2 cells. Int J Biochem Cell Biol, 2012. 44(11): p. 2019-27.
40. Saeed, A., et al., Disturbed Vitamin A Metabolism in Non-Alcoholic Fatty Liver Disease (NAFLD). Nutrients, 2017. 10(1).
41. Vu-Dac, N., et al., Transcriptional regulation of apolipoprotein A-I gene expression by the nuclear receptor RORalpha. J Biol Chem, 1997. 272(36): p. 22401-4.
42. Vu-Dac, N., et al., Retinoids increase human apolipoprotein A-11 expression through activation of the retinoid X receptor but not the retinoic acid receptor. Mol Cell Biol, 1996. 16(7): p. 3350-60.
43. Canas, J. A., et al., Insulin resistance and adiposity in relation to serum beta-carotene levels. J Pediatr, 2012. 161(1): p. 58-64 el-2.
44. Canas, J. A., et al., Effects of Mixed Carotenoids on Adipokines and Abdominal Adiposity in Children: A Pilot Study. J Clin Endocrinol Metab, 2017. 102(6): p. 1983-1990.

### Example 3

Losing Fat You Don't want Using Food Components

People distribute body fat two ways: (i) chest and abdomen, (ii) hips and thighs.

To lose fat where it is concentrated, rub cream embodiments where on wants to lose fat. For example, (i) hips and thighs, (ii) lose cellulite—bumpy appearance; (iii) breasts and abdomen; (iv) abdomen.

Why is it so hard to lose weight? (i) the body controls weight and blood pressure; (ii) the control point can be unhealthy; (iii) safe foods can be used to control weight; (iv) the concept is food as medicine.

Citrus Fruit: Grapefruit

The Grape Fruit diet—1930's

½ grapefruit 3 times/day gave weight loss and helped prevent diabetes

Referring to Fujioka K et al. J Med Food; 2006; 9(1):49-54

Citrus Fruit: Oranges

The Moro orange—purple inside

food and marmalade

study with juice

more actives in the whole orange

we use whole sweet oranges

Weight Loss Medications can Give 5% More Body Weight Loss than a Placebo

Prescription obesity drugs 3% to 7.5% greater weight loss than placebo

Moro orange juice powder 3.4% at 3 months, and 5.1% at 6 months

Whole orange extract—more active ingredients better weight loss

See Coulter A A et al. Drugs. 2018; 78(11): 1113-32

Obesity in Children

14% to 20% of children 2 to 18 years are obese

A pill extract of fruits and vegetables

Over 6 months children lost weight and helped to prevent diabetes

Weight loss due to beta-carotene (Vitamin A).

Second study used mixed carotenoids which contained beta-carotene (Vitamin A).

See Canas J A et al. J Pediatr. 2012; 161:58-64, Canas J A et al. J Clin Endocrinol Metab. 2017; 102:1983-90

### Summary

Lose fat where you want to lose it

Cream now made from food components.

Food components can act like drugs, but safer

Orange juice extract gives more weight than many obesity drugs

Whole orange and beta-carotene (vitamin A) give greater weight loss without drug side effects

### Example 4

Our studies in primary human adipocytes show that naringenin, a citrus flavonoid, increases oxygen consumption rate and gene expression of UCP1, GLUT4, and CPT-1(3. We investigated the safety of naringenin, its effects on metabolic rate, and blood glucose and insulin responses in a single female subject with diabetes. The subject ingested 150 mg naringenin from an extract of whole oranges standardized to 28% naringenin three times/day for eight weeks, and maintained her usual food intake. Body weight, resting metabolic rate, respiratory quotient, and blood chemistry panel including glucose, insulin and safety markers were measured at baseline and after eight weeks. Adverse events were evaluated every two weeks. We also examined the involvement of PPARα, PPARγ, PKA, and PKG in the response of human adipocytes to naringenin treatment. Compared to baseline, body weight decreased by 2.3 kg. The metabolic rate peaked at 3.5% above baseline at one hour but there was no change in the respiratory quotient. Compared to baseline, insulin decreased by 18% but the change in glucose was not clinically significant. Other blood safety markers were within their reference ranges, and there were no adverse events. UCP1 and CPT1β mRNA expression was reduced by inhibitors of PPARγ and PPARγ, but there was no effect of PKA or PKG inhibition. We conclude that naringenin supplementation is safe in humans, reduces body weight and insulin resistance, and increases metabolic rate by PPARα and PPARγ activation. The effects of naringenin on energy expenditure and insulin sensitivity will be tested in a randomized controlled clinical trial.

### Introduction

In recent years, polyphenols, such as flavonoids, have emerged as a class of natural products shown to have anti-obesity and insulin sensitizing effects.¹ Naringenin is a flavonoid found mainly in citrus fruits and tomato.^{2, 3}Naringenin exemplifies the term “phytopharmaceutical,” which can refer to its property for alleviating the effects of disorders such as the metabolic syndrome.¹ In obese humans, ½ grapefruit (49 mg naringenin) three times daily for 8 or 12 weeks reduced body weight and waist circumference compared to the placebo group.^{4, 5} Rodent studies show that naringenin reduces diet-induced weight gain and improves glucose and lipid metabolism.⁶⁻⁹ In mice fed a high-fat diet supplemented with naringenin, increases in energy expenditure and activation of brown fat have been demonstrated.⁷⁻¹⁰ Our in vitro studies in differentiated human subcutaneous adipose-derived stem cells from overweight and obese female donors show that naringenin increases gene expression of uncoupling protein 1 (UCP1), carnitine palmitoyltransferase 1 beta (CPT1β), glucose transporter type 4 (GLUT4), carbohydrate responsive element binding protein (ChREBP), and peroxi some proliferator-activated receptor gamma coactivator 1-α/β, (PGC-1α/β).⁶ The regulation of these genes are important determinants of thermogenesis, whole body insulin sensitivity, and glucose homeostasis.^{11, 12}

Flavonoids occur naturally as glycosides which means that they are bound to different sugars. Hydrolysis of the sugar moiety by colonic bacteria releases the aglycone naringenin. Therefore, the aglycone form rarely occurs in significant amounts in natural foods.¹³ Exploring the therapeutic potential of naringenin in humans has been hindered by previous studies demonstrating that following ingestion of citrus juices or fruits, the circulating concentrations of naringenin are low. Pharmacokinetic studies of orange juice and fruit have produced serum concentrations of <1 μm whereas cell culture and animal studies have determined that 1-200 μm is needed to elicit a physiologic response. The aglycone release by colonic microbiota is the rate-limiting step in the absorption of naringenin.¹⁴ We have previously shown that an aqueous and ethanolic extract of whole sweet oranges (Citrus Sinensis) containing naringenin, the free aglycone form, which can be readily absorbed from the small intestine is present in human serum at concentrations sufficient to elicit a physiologic response.¹⁵ Without wishing to be bound by theory, naringenin supplementation for eight weeks can increase resting metabolic rate (RMR) and insulin sensitivity in humans. We also determined the mechanisms by which naringenin mediates thermogenesis and glucose metabolism in differentiated human adipose-derived stem cells (hADSC). This study determined the safety of multiple dosing of naringenin, and its effects on energy expenditure and glucose metabolism in a single subject with untreated diabetes.

Subject

A 53 year old African American female subject was recruited. The subject was a nonsmoker, had a self-reported history of diabetes, was not taking prescription medications, and did not regularly consume citrus fruits. The subject met the inclusion criteria of fasting blood glucose between 126 and 200 mg/dL. She had been prescribed metformin but had discontinued the medication due to gastrointestinal intolerance. Exclusion criteria consisted of known allergy to citrus fruit. The study was approved by the Pennington Biomedical Research Center (PBRC) Institutional Review Board. The participant provided written informed consent. Procedures were in accordance with PBRC's ethical standards.

Methods

We conducted an eight-week case study. The subject completed three visits to the clinic. Visits were performed in the morning after an overnight fast for at least eight hours where only water was permitted. At the baseline visit, upon arrival, weight and vital signs (blood pressure and heart rate) were measured. The subject completed a medical questionnaire and had a physical exam. Blood was collected for a chemistry panel (glucose, insulin, creatinine, potassium, uric acid, albumin, calcium, magnesium, creatine phosphokinase, alanine aminotransferase, alkaline phosphatase, iron, total cholesterol high density lipoprotein cholesterol [HDL-C], low density lipoprotein cholesterol [LDL-C], and triglycerides) and a complete blood count (CBC). Resting metabolic rate (RMR) was measured over five hours at baseline and at the end of eight weeks using the ventilated hood to collect expirated gases. The test required the subject to lie quietly in bed after ingesting a capsule containing 150 mg naringenin. To evaluate the subject's metabolic rate, oxygen consumed and the carbon dioxide given off were measured during the last thirty minutes of each hour. Following the baseline testing, the subject was given 100 capsules each containing 150 mg naringenin with instructions to take one orally three times daily.

At weeks two and six, the subject received a phone call from the study coordinator and was asked about her progress and compliance. Changes in medications and adverse events were also evaluated. She was encouraged to comply with the study protocol. At week four, the subject returned to the clinic and her weight, blood pressure, and heart rate were measured. Any remaining capsules were collected and compliance was assessed. Naringenin (100 capsules) for the next four weeks was dispensed. Week eight marked the subject's last visit and involved repeating the baseline testing. At this visit, the subject returned any remaining capsules, and compliance was assessed.

Whole Orange Extract

Whole sweet oranges (Citrus Sinensis) were subjected to an aqueous and ethanolic extraction process, dried, milled, and provided in a powder form by Green Chem/Gencor Lifestage Solutions (Irvine, Calif.). The quantification of naringenin in the extract, and the safety and pharmacokinetics of the extract in humans are previously described.¹⁵ The extract contained 28% naringenin. Therefore each capsule prepared by the PBRC pharmacist contained 536 mg of the extract.

Human Subcutaneous Adipocyte Cell Culture

Abdominal adipose tissue stem cells from three female subjects (BMI: 27, 32, and 36 kg/m²) were obtained. Cells were seeded into culture plates and differentiated into mature adipocytes as previously described.⁶

The whole orange extract was dissolved in DMSO at 15 mM (based on 28% naringenin content) and added to cells at a dilution of 1:500 to achieve a final concentration of 30 μM. The inhibitors added to the naringenin-treated cells included: peroxisome proliferator-activated receptor alpha (PPARα) antagonist GW6471 and peroxisome proliferator-activated receptor gamma (PPARγ) antagonist GW9662 (Cayman Chemical, Ann Arbor, Mich.) at 10 μM concentration, protein kinase A (PKA) inhibitor H89 (Millipore Sigma, Burlington, Mass.) at 20 μM concentration, and protein kinase G (PKG) inhibitor Rp-8-pCPT-ck (Biolog Life Science Institute, AXXORA, LLC, Farmingdale, N.Y.) at 50 μM concentration. GW6471 and GW9662 were dissolved in DMSO at 1000×, H89 was dissolved in PBS at 500×, and Rp-8-pCPT-cGMPS was dissolved in PBS at 100× prior to dilution in cell culture medium. Following a two-day treatment, the cells were harvested with Trizol reagent. RNeasy Mini Kit (Qiagen, Germantown, Md.) was used to isolate total RNA following manufacturer's protocol. The expression of UCP1, CPT1β, and GLUT4 was quantified with one-step reverse transcriptase PCR using the reverse PCR primer to prime cDNA synthesis as previously described.⁶

Statistical Analysis of mRNA Expression

A general linear model was used to perform analysis of variance (ANOVA). The primary outcomes which were differences from the control and inhibitors were analyzed after Welch's test of homogeneity of variances. The assumption of normality was assessed using the Shapiro-Wilks test. Significance was set at p<0.05. Outcomes are summarized as means±SEM. Analyses were performed using SAS 9.4 (SAS Institute, Cary N. C.).

Results

Case Study

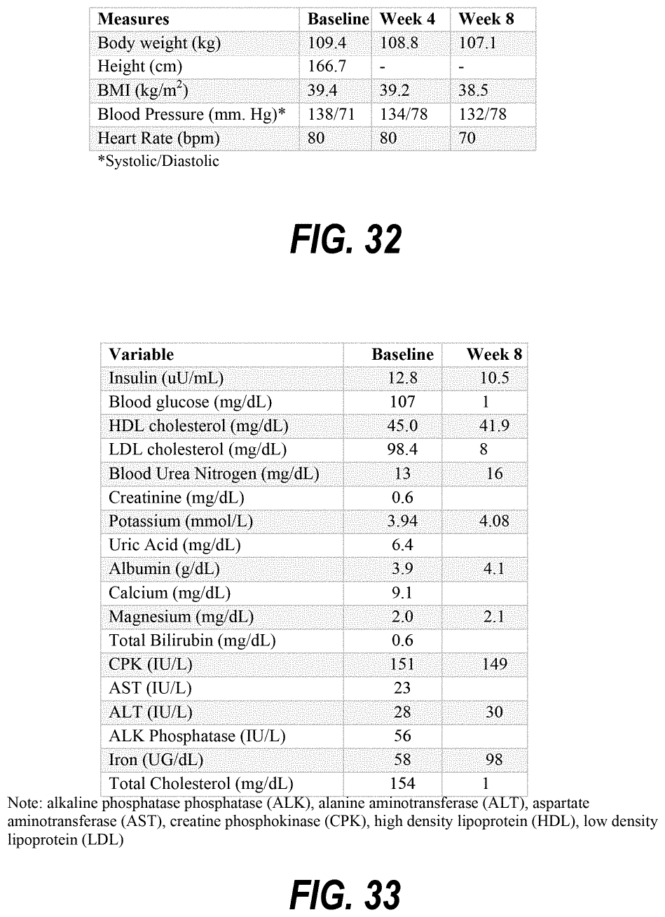

Weight and vital signs measured at baseline, week 4, and week 8 are shown in FIG. 32. Body weight decreased by 2.3 kg over the eight-week period. Body weight during a menstrual cycle cannot vary more than 1.5 kg, so the weight reduction of 2.3 kg is greater than the variability at a stable weight. Insulin decreased by 2.3 uU/mL (18%), and this change was greater than the 4.7% coefficient of variation (CV) of the assay. Serum glucose increased by 2 mg/dL (1.9%), which falls just outside the CV of the assay (1.6%). The homeostatic model assessment of insulin resistance (HOMA-IR) reduced from 3.4 at baseline to 2.8 (17.6%) at the end of the study.

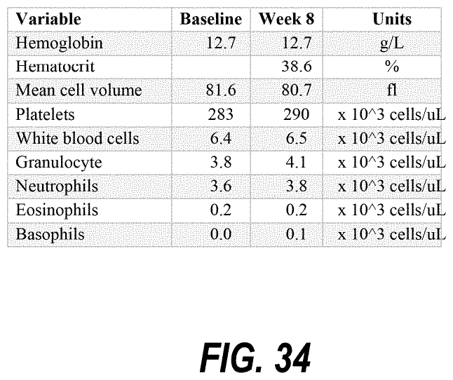

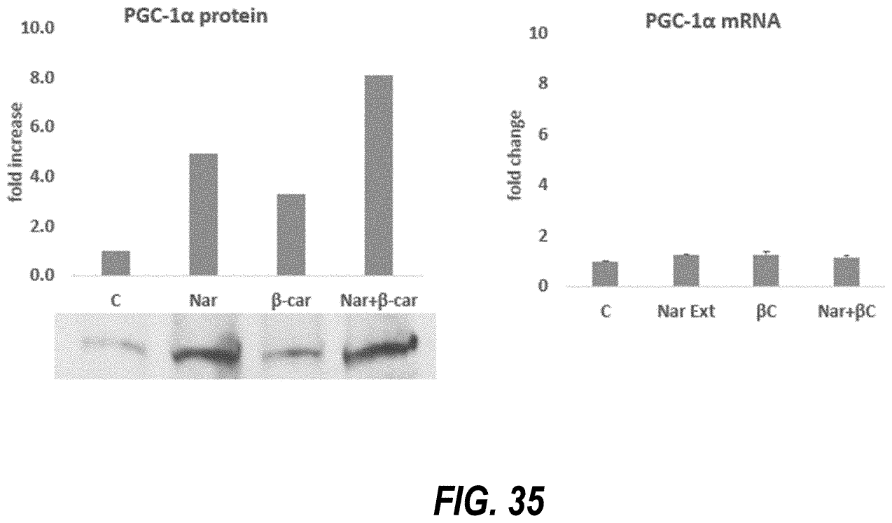

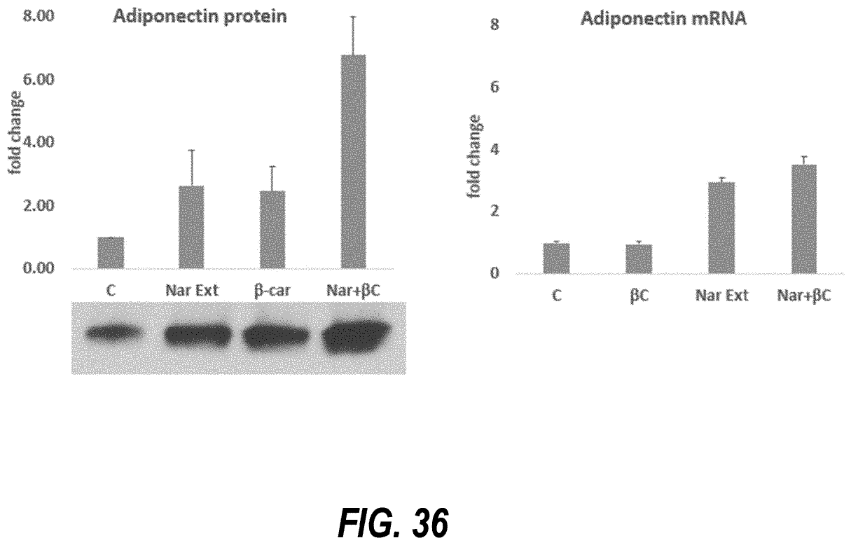

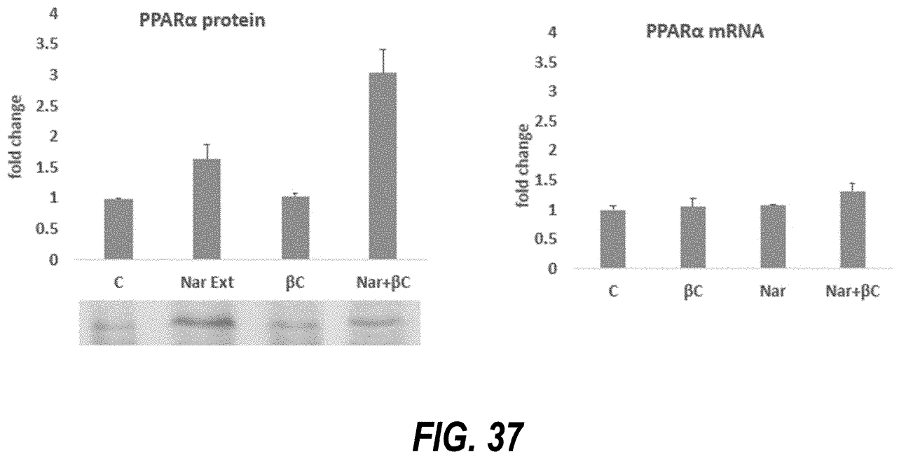

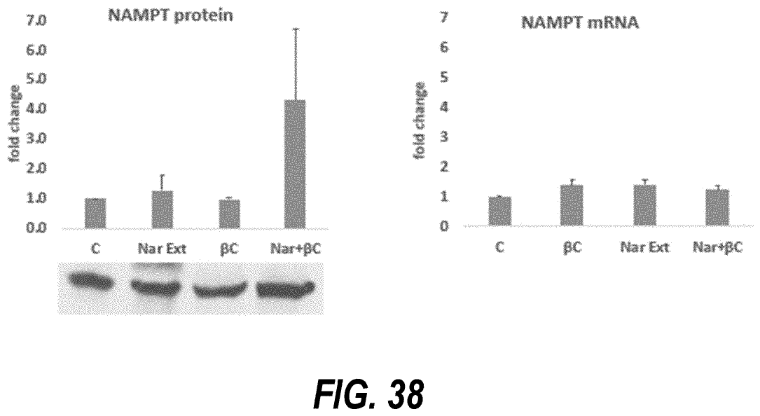

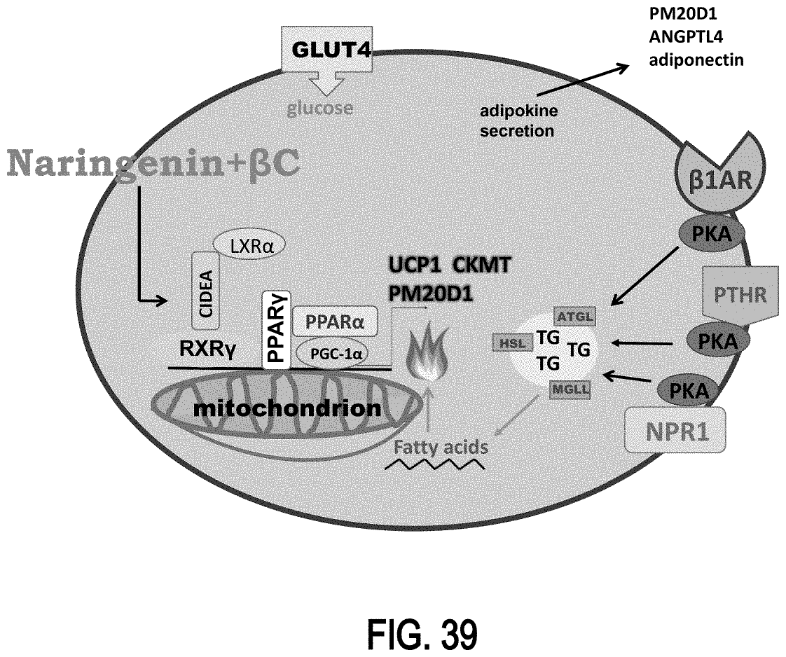

High density lipoprotein cholesterol decreased by 3.1 mg/dL (6.8%) which is greater than the assay CV of 1.6%. With the fall in HDL-C, there was a reciprocal change in triglycerides. The low triglyceride baseline value of 53 mg/dL led to an increase by 32% to 70 mg/d which is within the recommended value of <150 mg/dL Low density lipoprotein cholesterol decreased by 10.3 mg/dL, but this was a calculated value using the Friedewald equation and does not have a CV. The total cholesterol decreased by 10 mg/dL (6.5%) which is greater than the CV (1.2%) of the assay. The serum values of the blood markers assessed at baseline and after eight weeks of naringenin treatment are shown in FIG. 33 and FIG. 34.

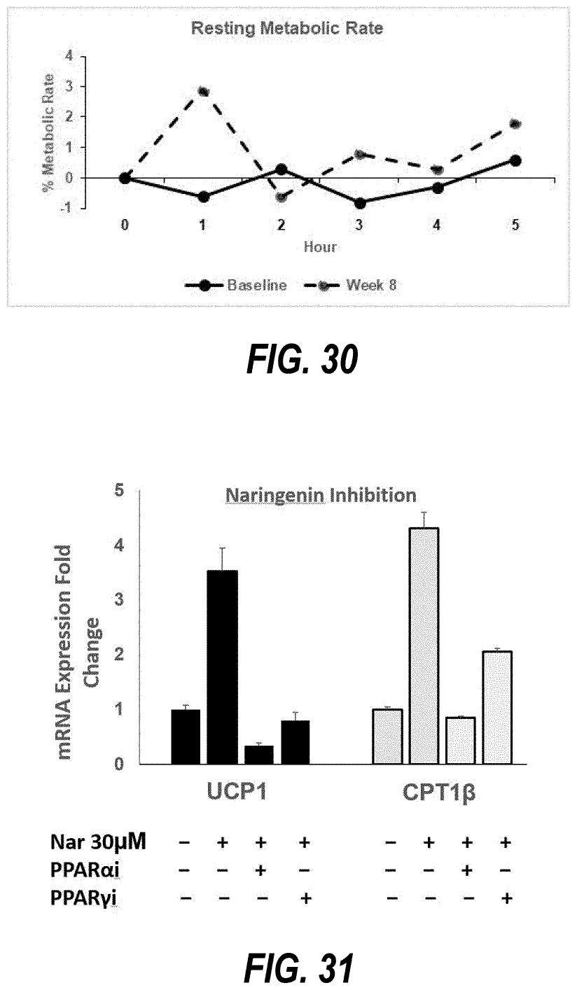

The RMR peaked at 3.5% above baseline at one hour (FIG. 30) and the maximal respiratory quotient increase was 1.2% above baseline at one hour. The CV of the metabolic rate for the metabolic cart used in this case study was 2.5% and the CV for the respiratory quotient was 1.5% to 2%. Thus, the metabolic rate increase was greater than the variability of the assay while the respiratory quotient did not change. No adverse events were reported. There were no clinically significant changes in blood safety markers (FIG. 33).

Gene Expression in Human Adipocytes

The mRNA expression of UCP1 and CPT1β increased by more than three-fold compared to control. The addition of PPARα and PPARγ inhibitors reduced the induction of UCP1 and CPT1β (p<0.001) over the course of the two-day treatment period (FIG. 2). PKA and PKG inhibition did not affect the mRNA expression of UCP1 or CPT1β. We have previously demonstrated an increase in GLUT4 mRNA expression over a seven-day treatment of human adipocytes with naringenin.⁶ However, over a two-day period there was no increase in GLUT4 mRNA expression with naringenin treatment which precluded determination of the effect of the inhibitors.

### Discussion

This is the first study in humans to investigate the effect of naringenin supplementation for eight weeks on energy expenditure and glucose metabolism. Body weight decreased by 2.3 kg over the eight week period. Serum total cholesterol and insulin concentrations reduced; however, HDL-C also decreased. The change in glucose was not clinically significant. However, there was a reduction in HOMA-IR by 17.6%. The RMR increased above the baseline value at one hour but there was no change in the respiratory quotient. In human adipocytes treated with naringenin at physiologically attainable doses.¹⁵ UCP1 and CPT1β mRNA expression were downregulated following inhibition of PPARγ and PPARγ, but were not affected by PKA or PKG inhibition.

Reduction in body weight occurs with a reduction in energy intake or an increase in energy expenditure or both. The majority of the studies of naringenin supplementation for 12 weeks in obese mouse models of metabolic dysfunction found a reduction in body weight without a decrease in food intake as measured in standard caging, and improvements in insulin sensitivity and lipid metabolism.^{7, 16, 17} When measured in metabolic cage studies, naringenin supplementation in mice results in a small but significant increase in energy intake.^{18, 19} However, energy expenditure increases, which can explain the reduction in body weight.^{7, 16-19} Consistent with the rodent studies, the subject in the study maintained her usual food intake, but her body weight reduced and metabolic rate increased with eight weeks of naringenin supplementation. Although there was no change in serum glucose concentrations, the reduction in fasting insulin and HOMA-IR indicates improvements in insulin sensitivity.

A reduction in HOMA-IR of 0.13 is associated with a diet-induced weight loss of one kg.²⁰ Therefore in our study, the 2.3 kg weight loss can predict a 0.3 reduction in HOMA-IR. We showed a 0.6 reduction in HOMA-IR without any dietary restriction which indicates that naringenin acts largely through factors independent of weight loss. Our in vitro data provide evidence of the effects of naringenin on upregulation of ChREBPβ and GLUT4 expression in human adipocytes, which in rodent models is associated with regulation of whole body glucose homestasis.^{6, 11, 21} In humans, expression of ChREBP in white adipose tissue correlates with insulin sensitivity. ^{11,22,23} In our case study, the subject lost weight and there was a perceptible change in her metabolic rate. Without wishing to be bound by theory, the improvement in insulin sensitivity can be attributed to, alone or in combination, the effects of naringenin on body weight, metabolic rate, and upregulation of target genes.

In mice placed on a weight cycling protocol, weight regain was accompanied by a significant reduction in energy expenditure. With a high fat diet, metabolomics studies showed that naringenin, its metabolite apigenin, and bile acids were depleted and did not return to normal levels during the weight loss despite recovery of other metabolic derangements. Administration of naringenin to mice during the high fat diet attenuated weight gain, increased energy expenditure and upregulated the gene expression of UCP1, the key regulator of thermogenesis in brown adipose tissue.¹⁰ The upregulation of UCP1 gene expression has also been shown in white adipose tissue of mice treated with naringenin, but the results are not consistent.^{17, 18}

The mechanisms by which naringenin exerts its effects on energy expenditure, lipid metabolism and insulin sensitivity are not completely understood. In hADSC and primary human white adipose tissue, we have previously shown a robust increase in the genes involved in thermogenesis and glucose metabolism with naringenin treatment. Naringenin stimulated mRNA expression of UCP1, GLUT4, PGC-1α/β (the nuclear receptor co-activators involved in thermogenesis), adipose triglyceride lipase (ATGL) and CPT1β (key enzymes necessary for fat oxidation), and adiponectin (insulin-sensitizing adipokine), in addition to increasing oxygen consumption rate.⁶

In rodents, the browning of adipose tissue is largely stimulated by sympathetic activation of the β3 adrenergic receptor (AR) that activates a signaling pathway involving cyclic adenine monphosphate (cAMP) and PKA.²⁴ However, human adipocytes predominantly express β1- and β2-ARs which can also have a role in thermogenesic activity.^{25, 26} Although β3 AR agonist treatment has been shown to increase metabolic activity in humans,²⁷ direct evidence of substantial contribution of the β3-AR to the thermogenic program is currently lacking.²⁸⁻³⁰ Bypassing ARs by treating with forskolin, a direct activator of the cAMP/PKA pathway, increases the expression of UCP1 in hADSC.³¹ Natriuretic peptides can act through PKG in human adipocytes to phosphorylate the same targets as the β-ARs do when acting through PKA to stimulate energy expenditure.³² Additionally, PPAR ligands have been shown to promote the conversion of human white adipocytes to the brown-like phenotype, decrease body fat, and increase expression of the genes required for fat oxidation. ^{31, 33, 34} Thus, PPAR activators have relevance to human physiology.

To identify the signaling pathways activated by naringenin in human adipocytes, we investigated the effect of PPARα and PPARγ inhibition as well as PKA and PKG inhibition. Consistent with evidence to indicate that naringenin is a activator of PPARα and PPARγ,³⁵ inhibition of these nuclear receptor proteins reduced mRNA expression of UCP1 and CPT1β. Inhibition of PKA or PKG did not have a significant effect on naringenin-stimulated induction of UCP1 or CPT1β mRNA, indicating that naringenin cannot act through the adrenergic signaling pathway. Mirabegron, a β3 AR agonist produces a marked increase in metabolic rate in humans but is accompanied by cardiovascular side effects.^{27, 36} Naringenin supplementation for eight weeks did not induce any adverse events and can be a safe way to stimulate energy expenditure, but the effects warrant investigation in a randomized controlled trial.

In epidemiologic studies, high flavanone intake from oranges and grapefruits is associated with a cardioprotective effect, especially, a reduction in the risk of ischemic stroke³⁷⁻³⁹ and human intervention trials show that grapefruit consumption improves body composition, insulin sensitivity, blood pressure and circulating lipids.^{4, 5} Grapefruit has been shown to increase the bioavailability of orally administered drugs. Without wishing to be bound by theory, naringenin being polyphenolic and high in electrons, can inhibit cytochrome P450 enzymes and enhance the bioavailability of medications including statins. However, unlike in the rodent and in vitro studies, the results of in vivo studies in humans indicate that naringenin is not the main inhibitory compound in grapefruit.^{40, 41} The clinically active constituents responsible for the inhibition are the furanocoumarin derivatives in grapefruit.⁴² While grapefruit contains a high content of furanocoumarin derivatives, sweet oranges contain a very small amount of these derivatives.⁴³ Therefore, we used an extract of sweet oranges which is low in the inhibitors of cytochrome P450 enzymes.

This study can provide that naringenin can cause an increase in metabolic rate. In conclusion, naringenin increases metabolic rate and improves insulin sensitivity in a human subject with untreated diabetes, without a change in food intake. Our in vitro studies indicate that naringenin can act through PPARα and PPARγ agonism in humans.

References cited in this example
1. Mulvihill E E, Burke A C, Huff M W: Citrus Flavonoids as Regulators of Lipoprotein Metabolism and Atherosclerosis. Annu Rev Nutr. 2016; 36:275-299.
2. Erdman J W, Jr., Balentine D, Arab L, et al.: Flavonoids and heart health: proceedings of the ILSI North America Flavonoids Workshop, May 31-Jun. 1, 2005, Washington, D.C. J Nutr. 2007; 137(3 Suppl 1):718S-737S.
3. Kawaii S, Tomono Y, Katase E, Ogawa K, Yano M: Quantitation of flavonoid constituents in citrus fruits. J Agric Food Chem. 1999; 47(9):3565-3571.
4. Fujioka K, Greenway F, Sheard J, Ying Y: The effects of grapefruit on weight and insulin resistance: relationship to the metabolic syndrome. J Med Food. 2006; 9(1):49-54.
5. Dow C A, Going S B, Chow H H, Patil B S, Thomson C A: The effects of daily consumption of grapefruit on body weight, lipids, and blood pressure in healthy, overweight adults. Metabolism. 2012; 61(7): 1026-1035.
6. Rebello C J, Greenway F L, Lau F H, et al.: Naringenin Promotes Thermogenic Gene Expression in Human White Adipose Tissue. Obesity (Silver Spring). 2018.
7. Mulvihill E E, Allister E M, Sutherland B G, et al.: Naringenin prevents dyslipidemia, apolipoprotein B overproduction, and hyperinsulinemia in LDL receptor-null mice with diet-induced insulin resistance. Diabetes. 2009; 58(10):2198-2210.
8. Mulvihill E E, Assini J M, Sutherland B G, et al.: Naringenin decreases progression of atherosclerosis by improving dyslipidemia in high-fat-fed low-density lipoprotein receptor-null mice. Arterioscler Thromb Vasc Biol. 2010; 30(4):742-748.
9. Mulvihill E E, Huff M W: Antiatherogenic properties of flavonoids: implications for cardiovascular health. Can J Cardiol. 2010; 26 Suppl A:17A-21A.
10. Thaiss C A, Itav S, Rothschild D, et al.: Persistent microbiome alterations modulate the rate of post-dieting weight regain. Nature. 2016.
11. Herman M A, Peroni O D, Villoria J, et al.: A novel ChREBP isoform in adipose tissue regulates systemic glucose metabolism. Nature. 2012; 484(7394):333-338.
12. Shepherd P R, Kahn B B: Glucose transporters and insulin action—implications for insulin resistance and diabetes mellitus. N Engl J Med. 1999; 341(4):248-257.
13. Peterson J J, Dwyer J T, Beecher G R, et al.: Flavanones in oranges, tangerines (mandarins), tangors, and tangelos: a compilation and review of the data from the analytical literature. J Food Comp Anal. 2006; 19:S66-73.
14. Manach C, Morand C, Gil-Izquierdo A, Bouteloup-Demange C, Remesy C: Bioavailability in humans of the flavanones hesperidin and narirutin after the ingestion of two doses of orange juice. Eur J Clin Nutr. 2003; 57(2):235-242.
15. Rebello C J, Beyl R A, Lertora J L L, et al.: Safety and Pharmacokinetics of Naringenin: A Randomized, Controlled, Single Ascending Dose, Clinical Trial. Diabetes Obes Metab. 2019; In Press.
16. Assini J M, Mulvihill E E, Sutherland B G, et al.: Naringenin prevents cholesterol-induced systemic inflammation, metabolic dysregulation, and atherosclerosis in Ldlr(−)/(−) mice. J Lipid Res. 2013; 54(3):711-724.
17. Assini J M, Mulvihill E E, Burke A C, et al.: Naringenin prevents obesity, hepatic steatosis, and glucose intolerance in male mice independent of fibroblast growth factor 21. Endocrinology. 2015; 156(6):2087-2102.
18. Burke A C, Sutherland B G, Telford D E, et al.: Intervention with citrus flavonoids reverses obesity and improves metabolic syndrome and atherosclerosis in obese Ldlr(−/−) mice. J Lipid Res. 2018; 59(9):1714-1728.
19. Burke A C, Telford D E, Edwards J Y, Sutherland B G, Sawyez C G, Huff M W: Naringenin Supplementation to a Chow Diet Enhances Energy Expenditure and Fatty Acid Oxidation, and Reduces Adiposity in Lean, Pair-Fed Ldlr(−/−) Mice. Mol Nutr Food Res. 2019; 63(6):e1800833.
20. Badri N W, Flatt S W, Barkai H S, Pakiz B, Heath D D, Rock C L: Insulin Resistance Improves More in Women than In Men in Association with a Weight Loss Intervention. J Obes Weight Loss Ther. 2018; 8(1).
21. Abdul-Wahed A, Guilmeau S, Postic C: Sweet Sixteenth for ChREBP: Established Roles and Future Goals. Cell Metab. 2017; 26(2):324-341.
22. Eissing L, Scherer T, Todter K, et al.: De novo lipogenesis in human fat and liver is linked to ChREBP-beta and metabolic health. Nat Commun. 2013; 4:1528.
23. Kursawe R, Caprio S, Giannini C, et al.: Decreased transcription of ChREBP-alpha/beta isoforms in abdominal subcutaneous adipose tissue of obese adolescents with prediabetes or early type 2 diabetes: associations with insulin resistance and hyperglycemia. Diabetes. 2013; 62(3):837-844.
24. Fedorenko A, Lishko P V, Kirichok Y: Mechanism of fatty-acid-dependent UCP1 uncoupling in brown fat mitochondria. Cell. 2012; 151(2):400-413.
25. Parysow O, Mollerach A M, Jager V, Racioppi S, San Roman J, Gerbaudo V H: Low-dose oral propranolol could reduce brown adipose tissue F-18 FDG uptake in patients undergoing PET scans. Clin Nucl Med. 2007; 32(5):351-357.
26. Soderlund V, Larsson S A, Jacobsson H: Reduction of FDG uptake in brown adipose tissue in clinical patients by a single dose of propranolol. European journal of nuclear medicine and molecular imaging. 2007; 34(7):1018-1022.
27. Cypess A M, Weiner L S, Roberts-Toler C, et al.: Activation of human brown adipose tissue by a beta3-adrenergic receptor agonist. Cell Metab. 2015; 21(1):33-38.
28. Schiffelers S L, Blaak E E, Saris W H, van Baak M A: In vivo beta3-adrenergic stimulation of human thermogenesis and lipid use. Clin Pharmacol Ther. 2000; 67(5):558-566.
29. Vosselman M J, van der Lans A A, Brans B, et al.: Systemic beta-adrenergic stimulation of thermogenesis is not accompanied by brown adipose tissue activity in humans. Diabetes. 2012; 61(12):3106-3113.
30. Redman L M, de Jonge L, Fang X, et al.: Lack of an effect of a novel beta3-adrenoceptor agonist, TAK-677, on energy metabolism in obese individuals: a double-blind, placebo-controlled randomized study. J Clin Endocrinol Metab. 2007; 92(2):527-531.
31. Bogacka I, Ukropcova B, McNeil M, Gimble J M, Smith S R: Structural and functional consequences of mitochondrial biogenesis in human adipocytes in vitro. J Clin Endocrinol Metab. 2005; 90(12):6650-6656.
32. Sengenes C, Bouloumie A, Hauner H, et al.: Involvement of a cGMP-dependent pathway in the natriuretic peptide-mediated hormone-sensitive lipase phosphorylation in human adipocytes. J Biol Chem. 2003; 278(49):48617-48626.
33. Barquissau V, Beuzelin D, Pisani D F, et al.: White-to-brite conversion in human adipocytes promotes metabolic reprogramming towards fatty acid anabolic and catabolic pathways. Mol Metab. 2016; 5(5):352-365.
34. Ribet C, Montastier E, Valle C, et al.: Peroxisome proliferator-activated receptor-alpha control of lipid and glucose metabolism in human white adipocytes. Endocrinology. 2010; 151(1):123-133.
35. Goldwasser J, Cohen P Y, Yang E, Balaguer P, Yarmush M L, Nahmias Y: Transcriptional regulation of human and rat hepatic lipid metabolism by the grapefruit flavonoid naringenin: role of PPARalpha, PPARgamma and LXRalpha. PLoS One. 2010; 5(8):e12399.
36. Hainer V: Beta3-adrenoreceptor agonist mirabegron—a potential antiobesity drug? Expert Opin Pharmacother. 2016; 17(16):2125-2127.
37. Joshipura K J, Ascherio A, Manson J E, et al.: Fruit and vegetable intake in relation to risk of ischemic stroke. JAMA. 1999; 282(13):1233-1239.
38. Cassidy A, Rimm E B, O'Reilly E J, et al.: Dietary flavonoids and risk of stroke in women. Stroke. 2012; 43(4):946-951.
39. Yamada T, Hayasaka S, Shibata Y, et al.: Frequency of citrus fruit intake is associated with the incidence of cardiovascular disease: the Jichi Medical School cohort study. J Epidemiol. 2011; 21(3):169-175.
40. Edwards D J, Bernier S M: Naringin and naringenin are not the primary CYP3A inhibitors in grapefruit juice. Life Sci. 1996; 59(13):1025-1030.
41. Bailey D G, Kreeft J H, Munoz C, Freeman D J, Bend J R: Grapefruit juice-felodipine interaction: effect of naringin and 6′,7′-dihydroxybergamottin in humans. Clin Pharmacol Ther. 1998; 64(3):248-256.
42. Guo L Q, Fukuda K, Ohta T, Yamazoe Y: Role of furanocoumarin derivatives on grapefruit juice-mediated inhibition of human CYP3A activity. Drug Metab Dispos. 2000; 28(7):766-771.
43. Saita T, Fujito H, Mori M: Screening of furanocoumarin derivatives in citrus fruits by enzyme-linked immunosorbent assay. Biol Pharm Bull. 2004; 27(7):974-977.

### Example 5

Synergistic Regulation of Protein Levels in Human Adipocyte by Naringenin Extract and Beta Carotene.

Naringenin (8 μM) and βCarotene (2 μM) increase adiponectin, PGC1α and NAMPT at the protein level synergistically after treatment for 7 days.

PGC-1α

The PGC-1α protein has a very short half-life (0.5 hours) and is rapidly degraded in the proteasome after ubiquitination. Most studies of PGC-1α protein stability have been conducted in mice and in murine cells, and show that protein levels closely follow mRNA levels. Our data shows differential regulation between protein mRNA. Without wishing to be bound by theory, the observed increases in PGC-1α protein induced by narengenin extract and β-carotene are due to inhibition of protein degradation by a post-translational modification such as phosphorylation. Phosphorylation at certain residues can slow degradation (JBC 285 p40192 2010, Azar, Ubiquitin-proteosome dependent degradation of PGC1a). Degradation rates can be influenced by fatty acids or serum factors.

Adiponectin

Mechanism for synergy between naringenin extract and β-carotene to increase adiponectin protein: Activation of both PPARγ and AMPK can increase adiponectin protein levels in 3T3 adipocytes (naringenin extract increases P-AMPK and our data using a PPARγ inhibitor indicates that PPARγ activity by naringenin is required for the mRNA induction). In other studies, β-carotene levels have been positively correlated with adiponectin levels in plasma.

Western Blot methods

Cells treated with 80 μM naringenin, 2 μM β-carotene or untreated controls for seven days were lysed in RIPA buffer containing a cocktail of protease and phosphatase. TGX SDS-PAGE gels (7.5%™, BioRad) were used to separate 50 μg of solubilized protein per sample. Following transfer, nitrocellulose membranes were probed overnight at 4° C. with primary monoclonal antibodies against adiponectin, NAMPT, and PPARγ (Santa Cruz), PGC-1α monoclonal (4C1.3, EMDmillipore) and β-Actin (A5316, Sigma). HRP-linked anti-rabbit (12-348, Sigma) and anti-mouse (AP130P, Sigma) were used to detect specific antibody-antigen complexes. Proteins were visualized by chemiluminescence (Western Lightning Plus-ECL, PerkinElmer, Waltham, Mass.). Bands were quantified using ImageJ software and values shown are normalized to β-actin.

### Example 6

Naringenin and BC Coordinately Regulate Lipolysis and Thermogenesis in Human Adipocytes (or Lipolysis, EE and Weight Loss)

Upregulation of Lipolysis and Thermogenesis by Naringenin and BC in Human Adipocytes Results in Weight Loss

### Introduction

Obesity has tripled worldwide in less than 50 years and is rising in adolescents and children (WHO 2020). Accumulation of fat mass is a risk factor for other chronic diseases including type 2 diabetes and cardiovascular disease (Stefan, 2020). FDA approved obesity drugs can act on the CNS to decrease appetite. These drugs act can exhibit a range of off-target side effects (Coulter et al., 2018). There is a need for the development of obesity treatments that are safe for long-term use and can act on peripheral target tissues to elevate energy expenditure, fat oxidation and insulin sensitivity.

Peroxisome proliferator activator receptors (PPARs) are ligand-activated nuclear receptors enriched in metabolic tissues, and many genes controlling fat oxidation and insulin sensitivity have PPAR regulatory elements (PPRE). Synthetic PPARα and PPARγ agonists are activators of thermogenic genes and energy expenditure and have been an area of pharmaceutical research for the treatment of metabolic diseases. PPARγ is a molecule with multiple ligand binding sites and layers of regulation by phosphorylation and acetylation (Choi et al., 2014; Qiang et al., 2012). Depending on the type of ligand and its conformation after ligand binding, PPARγ can stimulate adipogenesis, insulin sensitivity or thermogenic genes (Choi et al., 2011). TZDs are a class of synthetic PPARγ activators approved by the FDA that can increase insulin sensitivity but can exhibit the undesirable effects of adipogenesis and weight gain (DePaoli et al., 2014). A number of pharmacological PPAR activators are being investigated as treatments for diabetes and dyslipidemia in clinical trials, however, many trials have been discontinued due to side effects outweighing limited drug efficacy (Hong et al., 2018).

Natural compounds from plants and foods have been used for thousands of years in cultures to treat metabolic disorders, and a number of them act as PPAR ligands (Rigano et al., 2017). Naringenin (NR), a polyphenol found in sweet oranges, activates transcriptional activity of PPARα and PPARγ and thermogenic energy expenditure in human subcutaneous adipocytes from individuals with obesity (Goldwasser et al., 2010; Rebello et al., 2019). We evaluated the pharmacokinetics of NR from an extract of whole oranges (Citrus Sinensis) in a single, ascending dose, randomized clinical trial. NR was present in serum at concentrations sufficient to elicit a physiologic response following ingestion of the lowest dose of 150 mg and no adverse events were observed at any dose (Rebello et al., 2020). In a case study of an individual with obesity and untreated diabetes, ingestion of 150 mg of naringenin three times a day for eight weeks reduced body weight by 2.5%, improved fasting insulin concentrations, and produced a measurable increase in energy expenditure (Murugesan et al., 2020).

We screened flavonoids and vitamins in combination with NR for the enhancement of the transformation of human white adipocytes into thermogenically active cells. Treatment with the carotenoid β-carotene (BC) and NR (NRBC) synergistically elevated expression of uncoupling protein 1 (UCP1) and additional genes for mitochondrial uncoupling and energy-dissipating futile cycles in comparison to NR alone. Mitochondrial energy expenditure is driven by lipolysis, and we show that NRBC-treated adipocytes can have elevated expression of lipolytic receptors, protein kinase A (PKA), lipases and agonist-stimulated lipolysis. We conducted a case study of an individual with obesity to evaluate the effects of eight weeks of NRBC supplementation on energy expenditure and glucose metabolism. Without wishing to be bound by theory, alterations in adipocyte physiology can contribute to weight loss, insulin sensitivity and increases in diet-induced thermogenesis.

NR and BC can Synergistically Induce Expression of Genes for Thermogenesis and Insulin Sensitivity

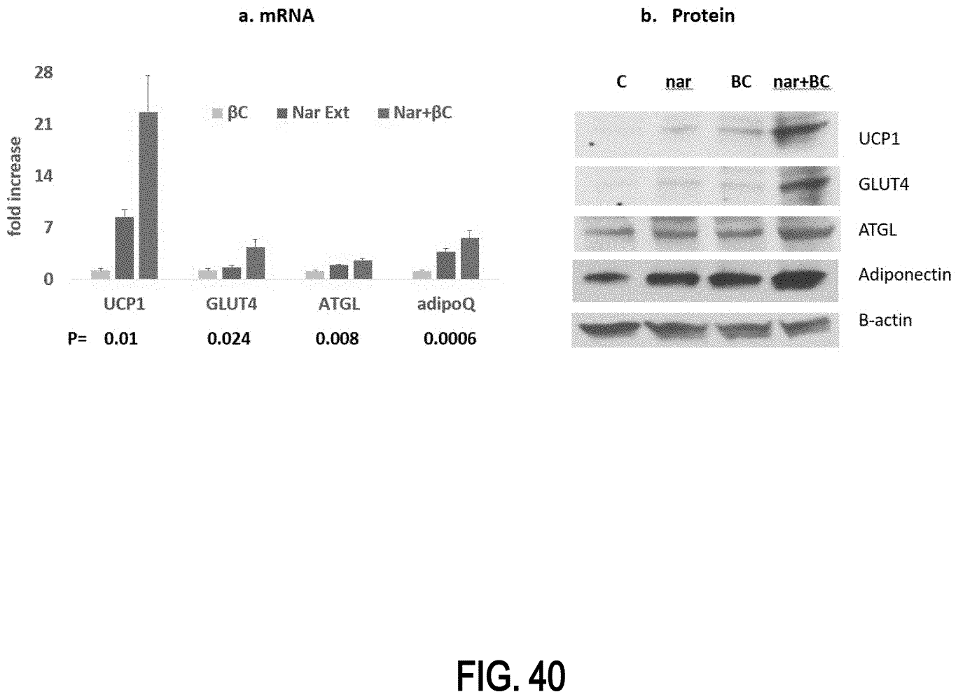

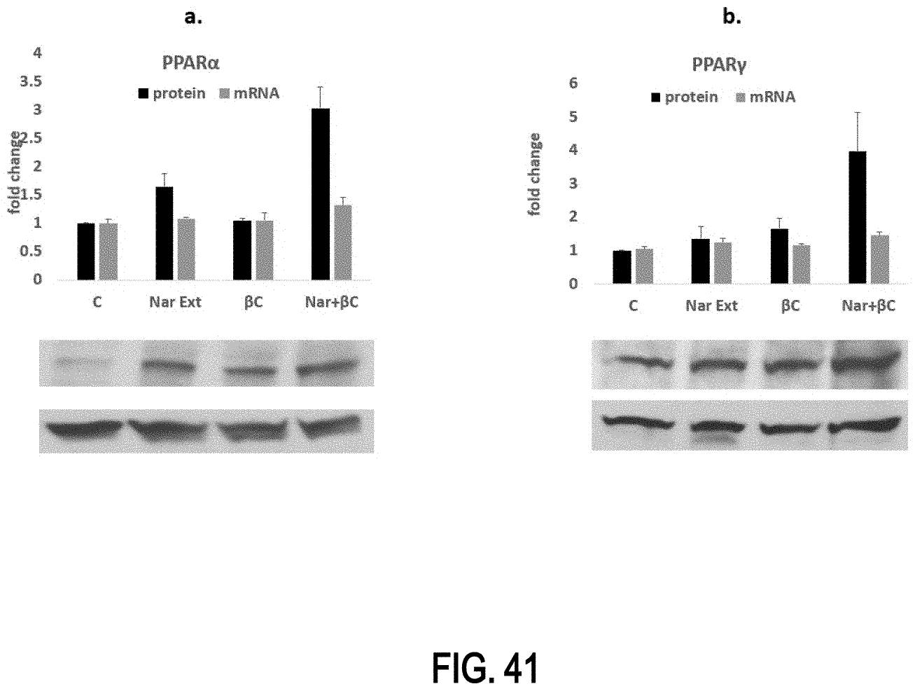

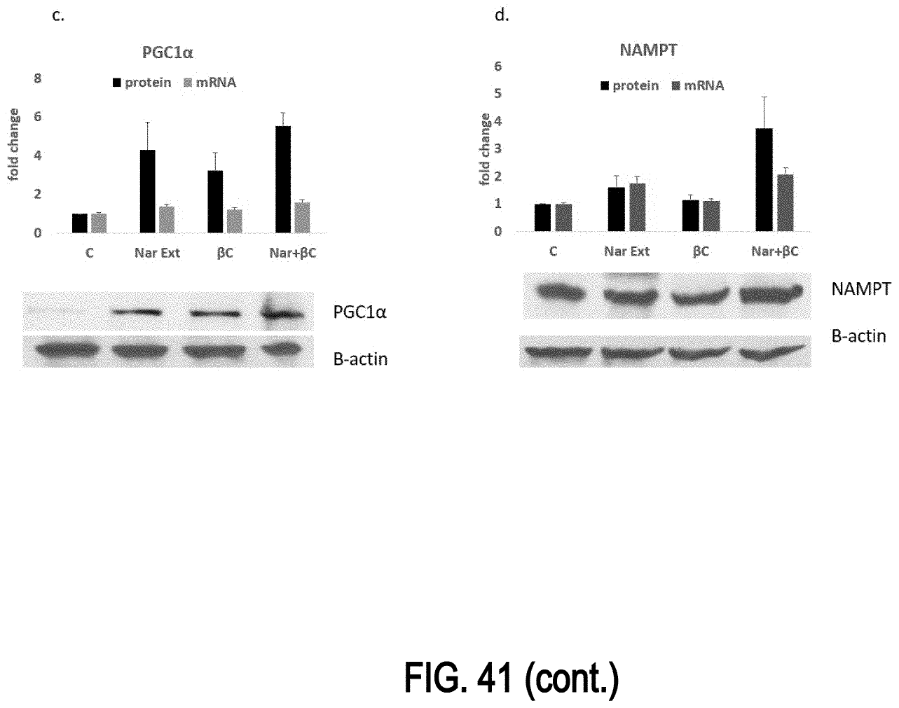

Carotenoids are vitamin A precursors found in foods and their consumption can cause reduction in adiposity and insulin resistance in pediatric clinical trials (Canas et al., 2012; Canas et al., 2017). After ingestion, they are metabolized into retinoid ligands for retinoic acid receptors (RAR) and retinoic X receptors (RXR) which are localized in the cell nucleus. RXRs form heterodimers with PPARs in transcriptional complexes known to target genes for energy expenditure and fat oxidation (Bonet et al., 2015). NR is a PPAR ligand, and we validated the effects of NR, BC and NRBC on gene expression in human adipocytes from five donors who were overweight or had obesity. A synergistic increase in mRNA levels for UCP1, CPT1b, ATGL and adiponectin occurred with NRBC in comparison to the individual ligands, and protein levels also trended higher for the combination treatment (FIG. 40). There was no change in the mRNA levels of PGC-1α, PPARα, PPARγ or NAMPT, but protein levels increased. Without wishing to be bound by theory, NRBC can regulate proteins through alternative, post-transcriptional mechanisms (FIG. 41). Two other carotenoids tested in combination with NR, lycopene and lutein, had a synergistic effect on gene expression that was similar to BC, and we chose BC for further analysis because of its safety and stability.

Whole Transcriptome Sequence Analysis of Changes in Gene Expression by NRBC

Metabolic Pathway Analysis

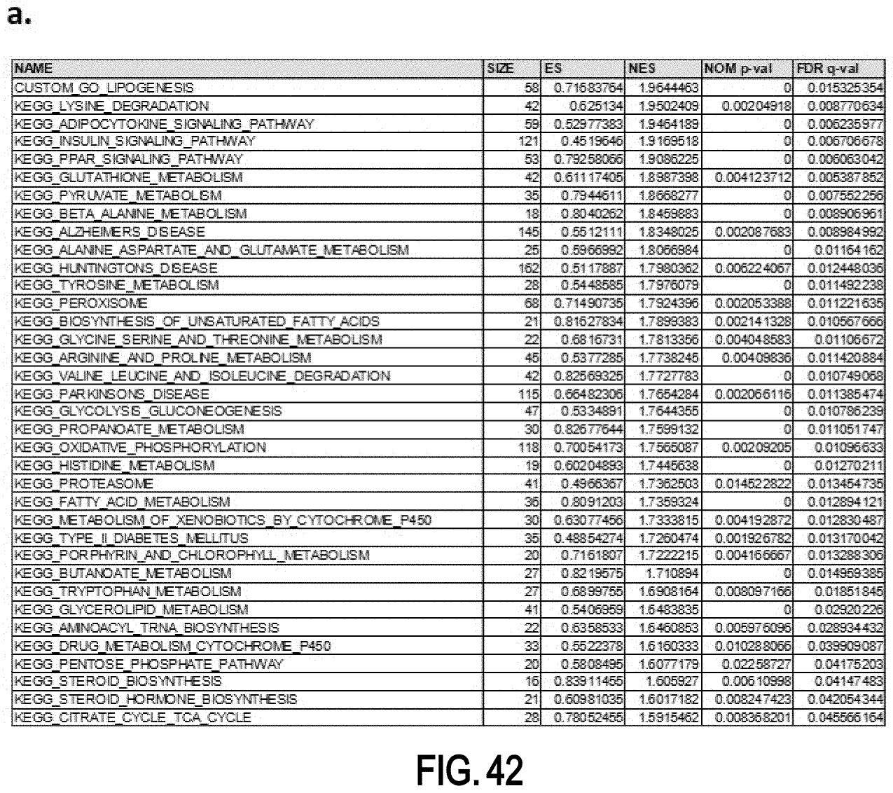

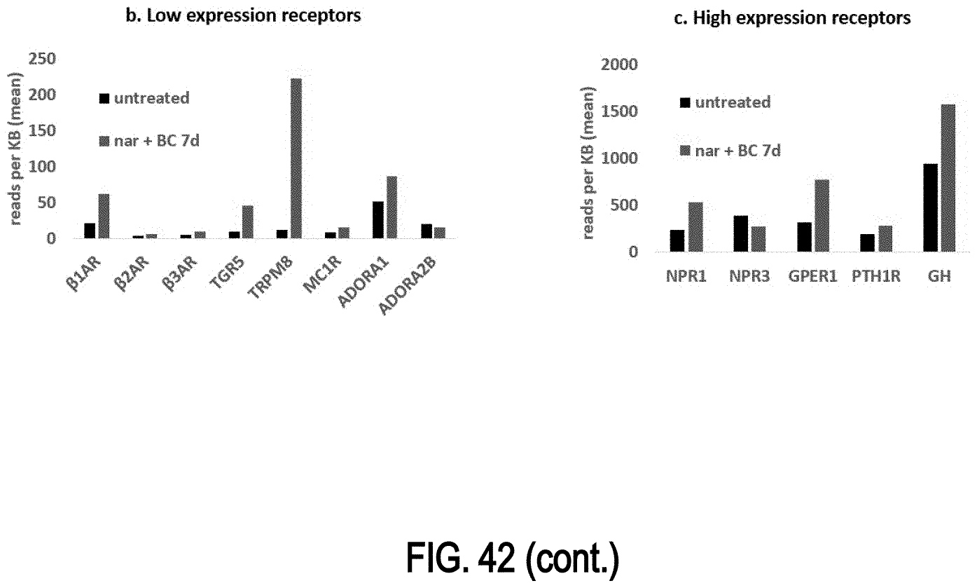

To expand our understanding of human adipocyte remodeling, we conducted transcriptome sequencing to evaluate NRBC-stimulated changes in the expressed genes. Adipocyte cultures from two female donors who had a body mass index (BMI) of 27 and 36 were treated with cell medium (vehicle control) or NRBC for seven days. Gene ontology analysis of the results showed that numerous genes for lipid, carbohydrate and amino acid metabolism were increased (FIG. 42 panel a).

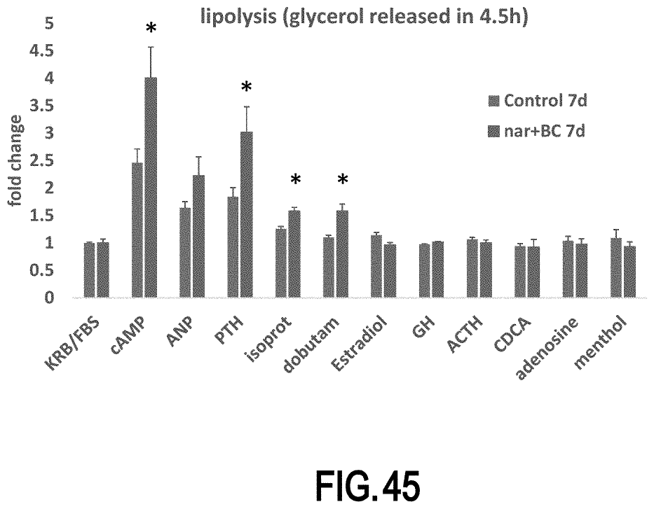

NRBC induced CIDEA, CITED, perilipins, PDK4, GK, AQP7, ELOVL3, ACSL5 and multiple FABPs (FIG. 45). See also, (Barquissau et al., 2016; Loft et al., 2015)

Adipose tissue is an endocrine organ that secretes proteins, hormones and bioactive lipids with beneficial paracrine effects on whole-body fat and glucose metabolism. NRBC treatment increased the expression of a number of these including ANGPTL4, NAD-producing enzymes NAMPT and NAPRT, GDF11, FNDC4, EPHX1 and EPHX2, and NMB (Fruhbeck et al., 2020; Katsimpardi et al., 2020; Ladenheim, 2010; McQueen et al., 2017; Vasan et al., 2019; Yamaguchi et al., 2019). Autocrine factors induced by NRBC included S100B, a stimulator of sympathetic innervation (Zeng et al., 2019) and CXCL14, a chemoattractant of thermogenesis-promoting M2 macrophages (Cereijo et al., 2018) (FIG. 45).

Induction of Thermogenic Genes for Non-UCP1 Uncoupling and Substrate Cycling

Several UCP1-independent mechanisms that can drive thermogenic energy expenditure have been identified in studies of BAT from cold exposed UCP1 knockout mice (reviewed in (Chang et al., 2019)). NRBC induced significant increases in a number of these genes in human adipocytes, and the changes were validated by qRT-PCR. PM20D1 was induced approximately 15-fold in adipocytes treated with NRBC. PM20D1 is an enzyme that regulates synthesis and degradation of N-acyl amino acids, and these molecules can uncouple mitochondria and increase energy expenditure (Long et al., 2016). PM20D1 is a secreted protein, and the actions of acyl amino acids in multiple tissues increase whole body energy expenditure and lower blood glucose in mice (Long et al., 2018). Human genetic studies have shown that the PM20D1 gene can be regulated by a PPRE and a high expression variant has been linked to neuroprotection against Alzheimer's disease (Benson et al., 2019; Sanchez-Mut et al., 2018).

Mitochondrial creatine kinases (CKMT) CKMT1A, CKMT1B and CKMT2 can be expressed in BAT in humans (Muller et al., 2016), and the three isozymes were induced by NRBC in white adipocytes. CKMTs enhance uncoupled energy expenditure by facilitating the availability of ADP and phosphate as substrates for ATP synthesis. Furthermore, NRBC upregulated levels of genes that can conduct futile cycling of triglycerides, PDK4 and PCK1, also a thermogenic process (Barquissau et al., 2016). These changes indicate that adipocytes have a higher capacity for both coupled and uncoupled respiration after NRBC exposure.

NRBC Increased Levels of Lipolysis-Linked Receptors, PKA and Triglyceride Lipases

The transformation of white adipocytes from fat storing to fat burning cells requires lipolysis to generate fatty acids for fuel along with substantial expression of thermogenic machinery in mitochondria (Yehuda-Shnaidman et al., 2010). The classical rodent model in which white adipose tissue plasticity was first characterized used cold exposure to drive sympathetic release of adrenergic agonists from nerve terminals, causing mitobiogenesis, lipolysis and UCP1 expression for thermoregulation (Young et al., 1984). These effects can be replicated by pharmacological treatment with agonists selective for the abundantly expressed β3 adrenergic receptor (β3AR) in rodent adipose tissue, and treatment of obese rodents with a β3AR agonist reduced fat mass (Collins et al., 1997; Himms-Hagen et al., 1994). Unfortunately, human adipocytes have low levels of βARs and clinical trials have shown that systemic administration of β3AR agonists can have little efficacy for weight loss and causes off-target cardiovascular adverse events (Cypess et al., 2015; Larsen et al., 2002; Redman et al., 2007). Without wishing to be bound by theory, a strategy is to upregulate expression of lipolytic receptors to heighten adipose tissue sensitivity to natural levels of lipolytic and thermogenic agonists. Accordingly, carotenoids promote expression of lipolytic receptors that participate in the human response to sympathetic stimuli such as cold (Bahouth et al., 1998; Karperien et al., 1999).

NRBC treatment significantly increased expression of eleven receptors which without wishing to be bound by theory can drive lipolysis through various mechanisms. We determined receptor abundances by comparing the number of transcript reads for each receptor (FIG. 42 panels b and c). The βSARs were the lowest compared to other lipolytic receptors; however, the three βARs increased over 2-fold after NRBC exposure. The β1AR was about 5-fold more abundant than the β2AR and β3AR, consistent with recent reports that β1AR is the predominant modulator of sympathetic stimulation in human adipocytes (Mattsson et al., 2011; Riis-Vestergaard et al., 2020).

Additional receptors that were expressed at low levels and significantly induced by NRBC were TGR5, TRPM8, ADORA1 and MC1R (FIG. 42 panel b). Ligands for TGR5 and TRPM8 induce UCP1 expression in human brown adipocytes and white adipocytes, respectively (Broeders et al., 2015; Rossato et al., 2014). Although NRBC did not alter ADORA2 levels, GAS2L2, a protein that increases the lipolytic activity of ADORA2, was upregulated over 20-fold (Wu et al., 2013). Not everything is known about the function of MC1R in human adipocytes, but its ligand ACTH is a key thermogenic hormone in mice (Hoch et al., 2007; Schnabl et al., 2018). MRAP, an accessory protein for melanocortin receptors that enhances ACTH-stimulated lipolysis in mice, was also elevated (Chan et al., 2009; Zhang et al., 2018).

NRBC upregulated a number of receptors expressed at high abundance including NPR1, PTH1R, GPER1 and GHR (FIG. 42 panel c). A ligand for NPR1 is atrial natriuretic peptide (ANP) and it is released from atrial myocytes in response to increases in blood pressure and environmental stimuli such as exercise and cold exposure (Carper et al., 2020; Moro et al., 2004). ANP acts additively with adrenergic agonists to stimulate lipolysis and brown adipocyte characteristics in human white adipocytes. The ratio of stimulatory NPR1 to the clearance receptor NPR3 can determine the extent of the response (Bordicchia et al., 2012). Transcript sequence data showed that the NPR1/NPR3 ratio improved four-fold in NRBC-treated adipocytes (FIG. 42 panel c).

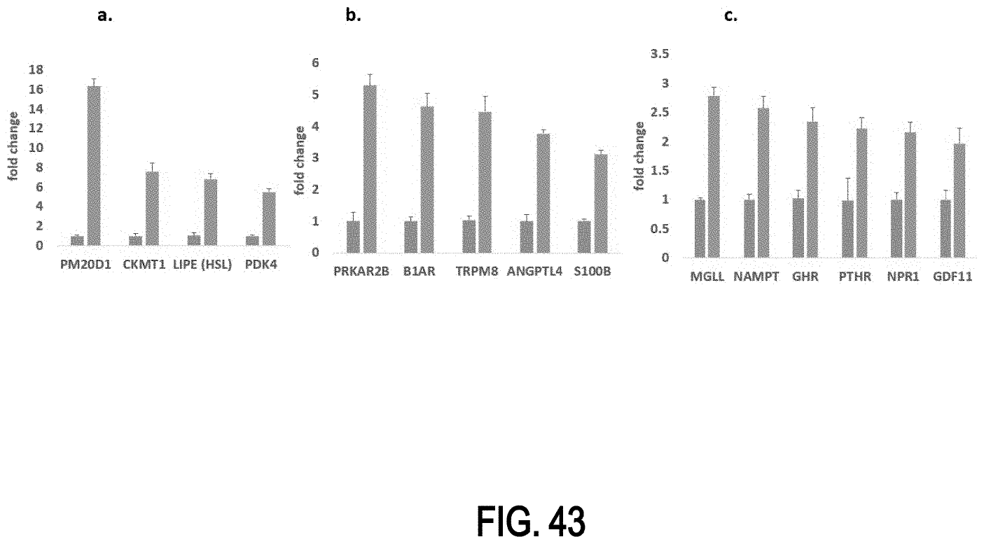

PTHR levels were upregulated over two-fold by NRBC (FIG. 43 panel c). Without wishing to be bound by theory, parathyroid hormone (PTH) plays a role in the human cold response. PTH stimulates thermogenic gene expression in adipocytes, and a rise in circulating concentrations of PTH occurs after cold exposure and correlates with a whole-body preference for lipids to fuel energy expenditure (Hedesan et al., 2019; Kovanicova et al., 2020).

GHR was the most abundant receptor upregulated by NRBC, and without wishing to be bound by theory, GH triggers lipolysis by unique mechanisms in human adipocytes (Balaz et al., 2015) (Sharma et al., 2019). Estradiol, the ligand for GPER1, regulates mitochondrial function in many tissues, but little is known about the role of GPER1 in adipocytes (Klinge, 2020; Prossnitz and Barton, 2014). Ligands for eleven receptors were chosen for functional evaluation based on their reported capability to stimulate lipolysis or energy expenditure in BAT or WAT.

The βARs, PTHR, MC1R and ADORA2 are Gs-coupled and signal through cAMP production and subsequent protein kinase A (PKA) activation to stimulate downstream lipase activity. We observed that NRBC significantly increased expression of PRKAR2B, the key PKA regulatory subunit linked to stimulated lipolysis, insulin sensitivity and resistance to weight gain in humans (Mantovani et al., 2009). Two additional catalytic and regulatory PKA subunits, PRKACA and PRKAR2A, were also elevated. The three lipases required for complete hydrolysis of fatty acids from the glycerol backbone of triglycerides, PNPLA2 (ATGL), LIPE (hormone sensitive lipase) and MGLL (monoglyceride lipase), also increased significantly (FIG. 43 panel a and 43 panel c). NPR1 is not a Gs coupled receptor and the signaling mechanism involves PKG activation, but there was no change in PKG expression.

Upregulation of Agonist-Stimulated Lipolysis:

Thermogenic energy expenditure is driven by lipolysis, so next we studied whether lipolysis levels in NRBC-treated adipocytes reflected the increases in receptor abundance. First, adipocyte cultures from four donors who were overweight or had obesity were treated with adipocyte medium (untreated control cells) or NRBC for 7 days. On the day of the acute lipolysis assay, cell cultures were rinsed with KRB to remove serum and exposed to the individual receptor agonists in KRB. Lipolysis was measured as glycerol released into the cell supernatant over 4.5 hour of agonist exposure. Non-hydrolyzable cAMP was also evaluated in each experiment as a positive control to measure the maximum capacity for PKA-stimulated lipolysis.

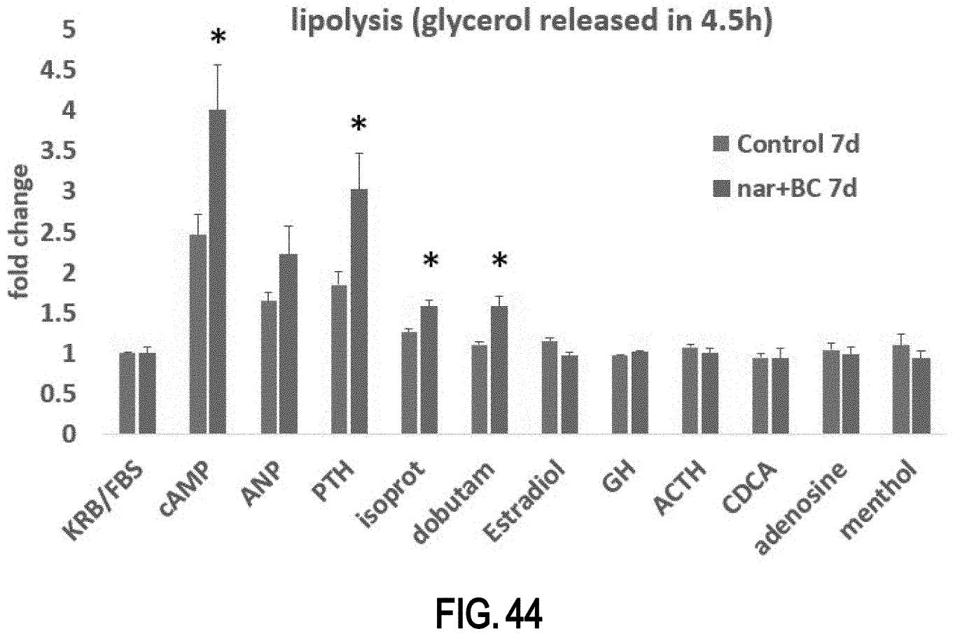

PTH and ANP stimulated the highest levels of lipolysis of receptor ligands (FIG. 44). PTH boosted lipolysis by the greatest magnitude in cultures exposed to NRBC relative to control cultures (FIG. 44). ANP-stimulated lipolysis trended higher in NRBC-treated cells, but the effect was variable among adipocytes from the four donors.

The collective activity of the βARs was measured using isoproterenol, a non-selective agonist of the three βARs, to evaluate the overall potential for increased sensitivity to sympathetic stimulation. Since the β1AR was 5-fold more abundant than the other βARs, the β1AR-selective ligand dobutamine was also tested. Isoproterenol- and dobutamine-stimulated lipolysis were significantly boosted in NRBC-treated cells, and dobutamine activity was comparable to isoproterenol (FIG. 44). Agonists of the other low expression receptors GPER, GHR, MC1R, ADORA1/ADORA2B, TGR5 and TRPM8 did not stimulate detectable increases in lipolysis over basal levels.

Cyclic AMP, which bypasses receptors to activate PKA, stimulated greater lipolysis activity than any individual receptor ligand. In NRBC-treated adipocytes, cAMP-stimulated lipolysis was significantly higher than levels in control cells, consistent with the upregulation of PKA regulatory and catalytic subunits. A mechanism to enhanced cAMP-stimulated lipolysis can be important because individuals with obesity have reduced TG lipolysis and removal rates and higher lipid storage rates. TG removal rate positively correlates with cAMP-stimulated lipolysis. (Amer et al., 2011). In a comprehensive 10-year follow-up study, subjects that had higher cAMP- or catecholamine-stimulated lipolysis in subcutaneous fat were weight stable over time (Amer et al., 2018). The subjects who gained weight over time had lower expression of five genes at baseline, and PRKAR2B and HSL were among them. Exercise is the only intervention previously known to improve the reduction in lipolysis that occurs with weight gain or aging (Nicklas et al., 1997)(You et al., 2004).

Upregulation of receptors and agonist-stimulated lipolysis after NRBC treatment indicates that adipose tissue in an individual taking NRBC is more responsive to stimuli such as cold exposure, food intake and exercise. To validate whether NRBC administration translates into weight loss and whole body energy expenditure in vivo, we next conducted a case study of an individual who was administered NRBC daily for eight weeks.

Increases in Energy Expenditure, Insulin Sensitivity and Weight Loss in an Individual with Obesity

Obesity is increasing in younger populations, and safe therapeutics are needed that can be taken over a lifetime (Skinner et al., 2018). This study indicates that NRBC acts by increasing the responsiveness of adipose tissue to natural receptor ligands that stimulate lipolysis, thermogenic energy expenditure and mobilization of adipokines. A case study of an individual with obesity and diabetes given NRBC for eight weeks.

### References Cited in this Example

Arner, P., Andersson, D. P., Backdahl, J., Dahlman, I., and Ryden, M. (2018). Weight Gain and Impaired Glucose Metabolism in Women Are Predicted by Inefficient Subcutaneous Fat Cell Lipolysis. Cell Metab 28, 45-54 e43.
Arner, P., Bernard, S., Salehpour, M., Possnert, G., Liebl, J., Steier, P., Buchholz, B. A., Eriksson, M., Arner, E., Hauner, H., et al. (2011). Dynamics of human adipose lipid turnover in health and metabolic disease. Nature 478, 110-113.
Bahouth, S. W., Beauchamp, M. J., and Park, E. A. (1998). Identification of a retinoic acid response domain involved in the activation of the beta 1-adrenergic receptor gene by retinoic acid in F9 teratocarcinoma cells. Biochem Pharmacol 55, 215-225.
Balaz, M., Ukropcova, B., Kurdiova, T., Vlcek, M., Surova, M., Krumpolec, P., Vanuga, P., Gasperikova, D., Klimes, I., Payer, J., et al. (2015). Improved adipose tissue metabolism after 5-year growth hormone replacement therapy in growth hormone deficient adults: The role of zinc-alpha2-glycoprotein. Adipocyte 4, 113-122.
Barquissau, V., Beuzelin, D., Pisani, D. F., Beranger, G. E., Mairal, A., Montagner, A., Roussel, B., Tavernier, G., Marques, M. A., Moro, C., et al. (2016). White-to-brite conversion in human adipocytes promotes metabolic reprogramming towards fatty acid anabolic and catabolic pathways. Mol Metab 5, 352-365.
Benson, K. K., Hu, W., Weller, A. H., Bennett, A. H., Chen, E. R., Khetarpal, S. A., Yoshino, S., Bone, W. P., Wang, L., Rabinowitz, J. D., et al. (2019). Natural human genetic variation determines basal and inducible expression of PM20D1, an obesity-associated gene. Proc Natl Acad Sci USA 116, 23232-23242.
Bonet, M. L., Canas, J. A., Ribot, J., and Palou, A. (2015). Carotenoids and their conversion products in the control of adipocyte function, adiposity and obesity. Arch Biochem Biophys 572, 112-125.
Bordicchia, M., Liu, D., Amri, E. Z., Ailhaud, G., Dessi-Fulgheri, P., Zhang, C., Takahashi, N., Sarzani, R., and Collins, S. (2012). Cardiac natriuretic peptides act via p38 MAPK to induce the brown fat thermogenic program in mouse and human adipocytes. J Clin Invest 122, 1022-1036.
Broeders, E. P., Nascimento, E. B., Havekes, B., Brans, B., Roumans, K. H., Tailleux, A., Schaart, G., Kouach, M., Charton, J., Deprez, B., et al. (2015). The Bile Acid Chenodeoxycholic Acid Increases Human Brown Adipose Tissue Activity. Cell Metab 22, 418-426.
Canas, J. A., Damaso, L., Altomare, A., Killen, K., Hossain, J., and Balagopal, P. B. (2012). Insulin resistance and adiposity in relation to serum beta-carotene levels. J Pediatr 161, 58-64 e51-52.
Canas, J. A., Lochrie, A., McGowan, A. G., Hossain, J., Schettino, C., and Balagopal, P. B. (2017). Effects of Mixed Carotenoids on Adipokines and Abdominal Adiposity in Children: A Pilot Study. J Clin Endocrinol Metab 102, 1983-1990.
Carper, D., Coue, M., Nascimento, E. B. M., Barquissau, V., Lagarde, D., Pestourie, C., Laurens, C., Petit, J. V., Soty, M., Monbrun, L., et al. (2020). Atrial Natriuretic Peptide Orchestrates a Coordinated Physiological Response to Fuel Non-shivering Thermogenesis. Cell Rep 32, 108075.
Cereijo, R., Gavalda-Navarro, A., Cairo, M., Quesada-Lopez, T., Villarroya, J., Moron-Ros, S., Sanchez-Infantes, D., Peyrou, M., Iglesias, R., Mampel, T., et al. (2018). CXCL14, a Brown Adipokine that Mediates Brown-Fat-to-Macrophage Communication in Thermogenic Adaptation. Cell Metab 28, 750-763 e756.
Chan, L. F., Webb, T. R., Chung, T. T., Meimaridou, E., Cooray, S. N., Guasti, L., Chapple, J. P., Egertova, M., Elphick, M. R., Cheetham, M. E., et al. (2009). MRAP and MRAP2 are bidirectional regulators of the melanocortin receptor family. Proc Natl Acad Sci USA 106, 6146-6151.
Chang, S. H., Song, N.J., Choi, J. H., Yun, U. J., and Park, K. W. (2019). Mechanisms underlying UCP1 dependent and independent adipocyte thermogenesis. Obes Rev 20, 241-251.
Choi, J. H., Banks, A. S., Kamenecka, T. M., Busby, S. A., Chalmers, M. J., Kumar, N., Kuruvilla, D. S., Shin, Y., He, Y., Bruning, J. B., et al. (2011). Antidiabetic actions of a non-agonist PPARgamma ligand blocking Cdk5-mediated phosphorylation. Nature 477, 477-481.
Collins, S., Daniel, K. W., Petro, A. E., and Surwit, R. S. (1997). Strain-specific response to beta 3-adrenergic receptor agonist treatment of diet-induced obesity in mice. Endocrinology 138, 405-413.
Coulter, A. A., Rebello, C. J., and Greenway, F. L. (2018). Centrally Acting Agents for Obesity: Past, Present, and Future. Drugs 78, 1113-1132.
Cypess, A. M., Weiner, L. S., Roberts-Toler, C., Franquet Elia, E., Kessler, S. H., Kahn, P. A., English, J., Chatman, K., Trauger, S. A., Doria, A., et al. (2015). Activation of human brown adipose tissue by a beta3-adrenergic receptor agonist. Cell Metab 21, 33-38.
DePaoli, A. M., Higgins, L. S., Henry, R. R., Mantzoros, C., Dunn, F. L., and Group, I. N. T. S. (2014). Can a selective PPARgamma modulator improve glycemic control in patients with type 2 diabetes with fewer side effects compared with pioglitazone? Diabetes Care 37, 1918-1923.
Fruhbeck, G., Fernandez-Quintana, B., Paniagua, M., Hernandez-Pardos, A. W., Valenti, V., Moncada, R., Catalan, V., Becerril, S., Gomez-Ambrosi, J., Portincasa, P., et al. (2020). FNDC4, a novel adipokine that reduces lipogenesis and promotes fat browning in human visceral adipocytes. Metabolism 108, 154261.
Goldwasser, J., Cohen, P. Y., Yang, E., Balaguer, P., Yarmush, M. L., and Nahmias, Y. (2010). Transcriptional regulation of human and rat hepatic lipid metabolism by the grapefruit flavonoid naringenin: role of PPARalpha, PPARgamma and LXRalpha. PLoS One 5, e12399.
Hedesan, O. C., Fenzl, A., Digruber, A., Spirk, K., Baumgartner-Parzer, S., Bilban, M., Kenner, L., Vierhapper, M., Elbe-Burger, A., and Kiefer, F. W. (2019). Parathyroid hormone induces a browning program in human white adipocytes. Int J Obes (Lond) 43, 1319-1324.
Himms-Hagen, J., Cui, J., Danforth, E., Jr., Taatjes, D. J., Lang, S. S., Waters, B. L., and Claus, T. H. (1994). Effect of CL-316,243, a thermogenic beta 3-agonist, on energy balance and brown and white adipose tissues in rats. Am J Physiol 266, R1371-1382.
Hoch, M., Eberle, A. N., Wagner, U., Bussmann, C., Peters, T., and Peterli, R. (2007). Expression and localization of melanocortin-1 receptor in human adipose tissues of severely obese patients. Obesity (Silver Spring) 15, 40-49.
Hong, F., Xu, P., and Zhai, Y. (2018). The Opportunities and Challenges of Peroxisome Proliferator-Activated Receptors Ligands in Clinical Drug Discovery and Development. Int J Mol Sci 19.
Karperien, M., Farih-Sips, H., Hendriks, J. A., Lanske, B., Papapoulos, S. E., Abou-Samra, A. B., Lowik, C. W., and Defize, L. H. (1999). Identification of a retinoic acid-inducible element in the murine PTH/PTHrP (parathyroid hormone/parathyroid hormone-related peptide) receptor gene. Mol Endocrinol 13, 1183-1196.
Katsimpardi, L., Kuperwasser, N., Camus, C., Moigneu, C., Chiche, A., Tolle, V., Li, H., Kokovay, E., and Lledo, P. M. (2020). Systemic GDF11 stimulates the secretion of adiponectin and induces a calorie restriction-like phenotype in aged mice. Aging Cell 19, e13038.
Klinge, C. M. (2020). Estrogenic control of mitochondrial function. Redox Biol 31, 101435.
Kovanicova, Z., Kurdiova, T., Balaz, M., Stefanicka, P., Varga, L., Kulterer, O. C., Betz, M. J., Haug, A. R., Burger, I. A., Kiefer, F. W., et al. (2020). Cold Exposure Distinctively Modulates Parathyroid and Thyroid Hormones in Cold-Acclimatized and Non-Acclimatized Humans. Endocrinology 161.
Ladenheim, E. E. (2010). To B or not to B: Fat is the question. J Physiol 588, 1393-1394.
Larsen, T. M., Toubro, S., van Baak, M. A., Gottesdiener, K. M., Larson, P., Saris, W. H., and Astrup, A. (2002). Effect of a 28-d treatment with L-796568, a novel beta(3)-adrenergic receptor agonist, on energy expenditure and body composition in obese men. Am J Clin Nutr 76, 780-788.
Loft, A., Forss, I., Siersbaek, M. S., Schmidt, S. F., Larsen, A. S., Madsen, J. G., Pisani, D. F., Nielsen, R., Aagaard, M. M., Mathison, A., et al. (2015). Browning of human adipocytes requires KLF11 and reprogramming of PPARgamma superenhancers. Genes Dev 29, 7-22.
Long, J. Z., Roche, A. M., Berdan, C. A., Louie, S. M., Roberts, A. J., Svensson, K. J., Dou, F. Y., Bateman, L. A., Mina, A. I., Deng, Z., et al. (2018). Ablation of PM20D1 reveals N-acyl amino acid control of metabolism and nociception. Proc Natl Acad Sci USA 115, E6937-E6945.
Long, J. Z., Svensson, K. J., Bateman, L. A., Lin, H., Kamenecka, T., Lokurkar, I. A., Lou, J., Rao, R. R., Chang, M. R., Jedrychowski, M. P., et al. (2016). The Secreted Enzyme PM20D1 Regulates Lipidated Amino Acid Uncouplers of Mitochondria. Cell 166, 424-435.
Mantovani, G., Bondioni, S., Alberti, L., Gilardini, L., Invitti, C., Corbetta, S., Zappa, M. A., Ferrero, S., Lania, A. G., Bosari, S., et al. (2009). Protein kinase A regulatory subunits in human adipose tissue: decreased R2B expression and activity in adipocytes from obese subjects. Diabetes 58, 620-626.
Mattsson, C. L., Csikasz, R. I., Chernogubova, E., Yamamoto, D. L., Hogberg, H. T., Amri, E. Z., Hutchinson, D. S., and Bengtsson, T. (2011). beta(1)-Adrenergic receptors increase UCP1 in human MADS brown adipocytes and rescue cold-acclimated beta(3)-adrenergic receptor-knockout mice via nonshivering thermogenesis. Am J Physiol Endocrinol Metab 301, E1108-1118.
McQueen, A. E., Kanamaluru, D., Yan, K., Gray, N. E., Wu, L., Li, M. L., Chang, A., Hasan, A., Stifler, D., Koliwad, S. K., et al. (2017). The C-terminal fibrinogen-like domain of angiopoietin-like 4 stimulates adipose tissue lipolysis and promotes energy expenditure. J Biol Chem 292, 16122-16134.
Moro, C., Crampes, F., Sengenes, C., De Glisezinski, I., Galitzky, J., Thalamas, C., Lafontan, M., and Berlan, M. (2004). Atrial natriuretic peptide contributes to physiological control of lipid mobilization in humans. FASEB J 18, 908-910.
Muller, S., Balaz, M., Stefanicka, P., Varga, L., Amri, E. Z., Ukropec, J., Wollscheid, B., and Wolfrum, C. (2016). Proteomic Analysis of Human Brown Adipose Tissue Reveals Utilization of Coupled and Uncoupled Energy Expenditure Pathways. Sci Rep 6, 30030.
Murugesan, N., Woodard, K., Ramaraju, R., Greenway, F. L., Coulter, A. A., and Rebello, C. J. (2020). Naringenin Increases Insulin Sensitivity and Metabolic Rate: A Case Study. J Med Food 23, 343-348.
Nicklas, B. J., Rogus, E. M., and Goldberg, A. P. (1997). Exercise blunts declines in lipolysis and fat oxidation after dietary-induced weight loss in obese older women. Am J Physiol 273, E149-155.
Prossnitz, E. R., and Barton, M. (2014). Estrogen biology: new insights into GPER function and clinical opportunities. Mol Cell Endocrinol 389, 71-83.
Qiang, L., Wang, L., Kon, N., Zhao, W., Lee, S., Zhang, Y., Rosenbaum, M., Zhao, Y., Gu, W., Farmer, S. R., et al. (2012). Brown remodeling of white adipose tissue by SirTl-dependent deacetylation of Ppargamma. Cell 150, 620-632.
Rebello, C. J., Beyl, R. A., Lertora, J. J. L., Greenway, F. L., Ravussin, E., Ribnicky, D. M., Poulev, A., Kennedy, B. J., Castro, H. F., Campagna, S. R., et al. (2020). Safety and pharmacokinetics of naringenin: A randomized, controlled, single-ascending-dose clinical trial. Diabetes Obes Metab 22, 91-98.
Rebello, C. J., Greenway, F. L., Lau, F. H., Lin, Y., Stephens, J. M., Johnson, W. D., and Coulter, A. A. (2019). Naringenin Promotes Thermogenic Gene Expression in Human White Adipose Tissue. Obesity (Silver Spring) 27, 103-111.
Redman, L. M., de Jonge, L., Fang, X., Gamlin, B., Recker, D., Greenway, F. L., Smith, S. R., and Ravussin, E. (2007). Lack of an effect of a novel beta3-adrenoceptor agonist, TAK-677, on energy metabolism in obese individuals: a double-blind, placebo-controlled randomized study. J Clin Endocrinol Metab 92, 527-531.
Rigano, D., Sirignano, C., and Taglialatela-Scafati, 0. (2017). The potential of natural products for targeting PPARalpha. Acta Pharm Sin B 7, 427-438.
Riis-Vestergaard, M. J., Richelsen, B., Bruun, J. M., Li, W., Hansen, J. B., and Pedersen, S. B. (2020). Beta-1 and Not Beta-3 Adrenergic Receptors May Be the Primary Regulator of Human Brown Adipocyte Metabolism. J Clin Endocrinol Metab 105.
Rossato, M., Granzotto, M., Macchi, V., Porzionato, A., Petrelli, L., Calcagno, A., Vencato, J., De Stefani, D., Silvestrin, V., Rizzuto, R., et al. (2014). Human white adipocytes express the cold receptor TRPM8 which activation induces UCP1 expression, mitochondrial activation and heat production. Mol Cell Endocrinol 383, 137-146.
Sanchez-Mut, J. V., Heyn, H., Silva, B. A., Dixsaut, L., Garcia-Esparcia, P., Vidal, E., Sayols, S., Glauser, L., Monteagudo-Sanchez, A., Perez-Tur, J., et al. (2018). PM20D1 is a quantitative trait locus associated with Alzheimer's disease. Nat Med 24, 598-603.
Schnabl, K., Westermeier, J., Li, Y., and Klingenspor, M. (2018). Opposing Actions of Adrenocorticotropic Hormone and Glucocorticoids on UCP1-Mediated Respiration in Brown Adipocytes. Front Physiol 9, 1931.
Sharma, V. M., Vestergaard, E. T., Jessen, N., Kolind-Thomsen, P., Nellemann, B., Nielsen, T. S., Vendelbo, M. H., Moller, N., Sharma, R., Lee, K. Y., et al. (2019). Growth hormone acts along the PPARgamma-FSP27 axis to stimulate lipolysis in human adipocytes. Am J Physiol Endocrinol Metab 316, E34-E42.
Skinner, A. C., Ravanbakht, S. N., Skelton, J. A., Perrin, E. M., and Armstrong, S. C. (2018). Prevalence of Obesity and Severe Obesity in US Children, 1999-2016. Pediatrics 141.
Stefan, N. (2020). Causes, consequences, and treatment of metabolically unhealthy fat distribution. Lancet Diabetes Endocrinol 8, 616-627.
Vasan, S. K., Noordam, R., Gowri, M. S., Neville, M. J., Karpe, F., and Christodoulides, C. (2019). The proposed systemic thermogenic metabolites succinate and 12,13-diHOME are inversely associated with adiposity and related metabolic traits: evidence from a large human cross-sectional study. Diabetologia 62, 2079-2087.
Wu, Y. C., Lai, H. L., Chang, W. C., Lin, J. T., Liu, Y. J., and Chern, Y. (2013). A novel Galphas-binding protein, Gas-2 like 2, facilitates the signaling of the A2A adenosine receptor. Biochim Biophys Acta 1833, 3145-3154.
Yamaguchi, S., Franczyk, M. P., Chondronikola, M., Qi, N., Gunawardana, S. C., Stromsdorfer, K. L., Porter, L. C., Wozniak, D. F., Sasaki, Y., Rensing, N., et al. (2019). Adipose tissue NAD(+) biosynthesis is required for regulating adaptive thermogenesis and whole-body energy homeostasis in mice. Proc Natl Acad Sci USA 116, 23822-23828.
Yehuda-Shnaidman, E., Buehrer, B., Pi, J., Kumar, N., and Collins, S. (2010). Acute stimulation of white adipocyte respiration by PKA-induced lipolysis. Diabetes 59, 2474-2483.
You, T., Berman, D. M., Ryan, A. S., and Nicklas, B. J. (2004). Effects of hypocaloric diet and exercise training on inflammation and adipocyte lipolysis in obese postmenopausal women. J Clin Endocrinol Metab 89, 1739-1746.
Young, P., Arch, J. R., and Ashwell, M. (1984). Brown adipose tissue in the parametrial fat pad of the mouse. FEBS Lett 167, 10-14.
Zeng, X., Ye, M., Resch, J. M., Jedrychowski, M. P., Hu, B., Lowell, B. B., Ginty, D. D., and Spiegelman, B. M. (2019). Innervation of thermogenic adipose tissue via a calsyntenin 3beta-S100b axis. Nature 569, 229-235.
Zhang, X., Saarinen, A. M., Campbell, L. E., De Filippis, E. A., and Liu, J. (2018). Regulation of Lipolytic Response and Energy Balance by Melanocortin 2 Receptor Accessory Protein (MRAP) in Adipocytes. Diabetes 67, 222-234.

### Example 7

### Case Study to Evaluate the Safety and Effect on Energy Expenditure of Naringenin and Beta Carotene

Objectives: To validate the safety and the effect on energy expenditure of an extract of sweet oranges given as one capsule three times a day, with beta carotene one capsule per day, over 8 weeks.

### Background

A project to validate new transcription factors for increasing energy expenditure in primary human adipocytes treated with the synergistic combination of naringenin and beta-carotene was awarded. The mRNA sequencing and bioinformatics analyses identified receptors as being upregulated, as well as well as upregulation of lipases and the thermogenic machinery of adipocyte mitochondria. This finding indicates an increase in the sensitivity of adipose tissue to the natural release of sympathetic norepinephrine and other hormones without the side effects caused by administration of systemic adrenergic agonists.

Systemic administration of adrenergic agonists in humans increases energy expenditure but can have adverse cardiovascular outcomes because of off target effects. Without wishing to be bound by theory, increases in multiple Gs-linked receptors in adipocytes can increase lipolysis and energy expenditure, but this effect will be specific to adipose tissue, because there were no adverse events in our pharmacokinetic clinical trial of an extract of sweet oranges containing naringenin¹ or in the prior case report testing the effect of naringenin alone.² Beta-carotene is the precursor for vitamin A in the human diet. It is an essential nutrient with a clear safety profile.

We can validate the effects of naringenin+beta-carotene in a human model as the beta adrenergic receptors in rodents work differently and do not translate to similar effects in humans. A human case study in the same person that participated in the case study of an extract of sweet oranges containing naringenin without beta-carotene can confirm a greater stimulation of energy expenditure from the higher dose of naringenin combined with beta carotene. The higher dose was shown to be safe in our safety and pharmacokinetic study of naringenin. It can also advance science by validating the discovery of a new pathway of B1 receptor agonism, and provide a hitherto unknown treatment for obesity and insulin resistance. The data in a human case study can advance this research.

A case study in the same patient who was included in the prior case study of naringenin alone can be conducted. The subject will be treated for 8 weeks with the combination of naringenin+beta carotene. We can conduct the following assessments before the treatment and at the end of eight weeks.
RMR for 4 hours (measured for 2 hours after taking the two supplements and for the second 2 hours after drinking 795 kcal (12 ounces) of Boost nutritional supplement
24-hour ambulatory blood pressure
3-hour OGTT with glucose and insulin (0, 1, 2, 3 hrs measurements)
Blood drawn for CBC, Chemistry 15 panel, and proteomics analysis

Inclusion and Exclusion Criteria

The inclusion and exclusion criteria are listed herein.

Inclusion Criteria:
Fasting blood glucose<200 mg/dL
On no medication for diabetes, weight loss, or that alters metabolic rate.

Exclusion Criteria:
Known allergy to citrus fruit.
Vulnerable populations like children, people unable to give consent, prisoners and females who are pregnant

Number of Subjects

This study can include one subject and can be published in a case report.

In a non-limiting example, the subject has type 2 diabetes. Subject had an unpleasant reaction to the metformin prescribed by her physician. Subject now takes no medication for diabetes.

Study Timelines

The study will occur over a period of eight weeks.

Procedures Involved

An exemplary, non-limiting example of the study can follow the schedule of events in Table 1:

| Test/Visit | Baseline a | Baseline b | Baseline c | Week 2 | Week 4 | Week 6 | Week 8 a | Week 8 b |
|---|---|---|---|---|---|---|---|---|
| Informed Consent | X | | | | | | | |
| Medical Questionnaire | X | | | | | | | |
| Brief Physical Exam | X | | | | | | | |
| Chemistry-15 & CBC/Proteomics | X | | | | | | | X |
| Proteomics | | | X | | | | | |
| 3 hour Oral Glucose Tolerance | X | | | | | | X | |
| Test* | | | | | | | | |
| 24 hour Blood Pressure monitor | X | | | | | | X | |
| 5-hour RMR** | | X | | | | | | X |
| Distribution of Supplements | | X | | | X | | | |
| Collection for pill counts | | | | | X | | X | |
| Adverse Events (phone) | | | | X | | X | | |
| Adverse Events | | X | | | X | | X | X |
| Medications | X | | | X | X | X | X | X |
| Height | X | | | | | | | |
| Weight, BP, Pulse Rate | X | | | | X | | X | |

Visits Baseline b and 8 b will occur the next day after visits Baseline a and 8 a respectively. Baseline visit 8 c will occur the day after 8 b
*With glucose and insulin
**Supplement after one hour and nutritional drink 2 hours later

Baseline Visit a: Approximately 4 Hours. Subject Will Fast from 9.00 p.m. The Prior Night Except for Water.

The subject will arrive to the clinic after fasting except for water from 9 p.m. the prior night. The subject will sign an informed consent after reading the consent form, having the study explained and after the questions are answered to her satisfaction. The subject will then complete a medical questionnaire, have a brief physical exam and measurements of height, weight, blood pressure and pulse rate will be completed. She will have blood drawn from and arm vein for glucose, insulin, a chemistry panel, complete blood count (CBC) and proteomics analysis. She will have a 3-hour glucose tolerance test with blood drawn for insulin and glucose at, 1, 2, and 3 hours. She will be fitted with a 24-hour blood pressure monitor.

Baseline Visit b: Approximately 6 Hours. Subject Will Fast from 9.00 p.m. The Prior Night Except for Water.

The subject will come to this visit fasting from 9.00 p.m. the prior night. She will return her 24-hour blood pressure monitor. She will have a 5-hour testing of her metabolic rate. Following the baseline testing two naringenin capsules containing 310 mg of naringenin in a 1 g extract of sweet orange (Citrus sinensis) and capsules containing 6 mg beta-carotene will be administered. After two hours, she will be asked to drink a nutrition supplement (Boost) and have her metabolic rate measured for another 2 hours. She will then be given a month's supply of naringenin 310 mg in 1 g of sweet orange extract capsules (200 capsules) with instructions to take two orally three times a day and a month's supply of beta carotene 6 mg (two capsules) to be taken twice daily (120 capsules).

Baseline c: Approximately Half-Hour. Subject Will Fast from 9.00 p.m. The Prior Night Except for Water

The subject will come to this visit fasting from 9.00 p.m. the prior night. Blood will be drawn for proteomics analysis.

Phone Visits at Week 2 and Week 6: Approximately 5 to 15 Minutes

The subject will receive a phone call at Week 2 and Week 6. The subject will be asked open-ended questions about how she is doing to determine whether any adverse events have occurred and to encourage her compliance with the instructions to take the naringenin capsules three times a day and the beta carotene capsules daily.

Week 4 Visit: Approximately 30 Minutes

The subject will come to the clinic at Week 4. She will have weight, blood pressure, and pulse rate recorded. The bottle with any remaining capsules will be collected and another month's supply of -naringenin and capsules dispensed. She will be asked about any adverse events using open-ended questions about how she is doing.

Week 8a Visit: Approximately 4 Hours. Subject Will Fast from 9.00 p. m. the Prior Night Except for Water.

The subject will arrive to the clinic after fasting except for water from 9.00 p.m. the prior night. Weight, blood pressure and pulse rate will be measured. She will have blood drawn from and an arm vein for glucose, insulin, a chemistry panel, a complete blood count (CBC) and proteomics analysis. She will have a 3-hour glucose tolerance test with blood drawn for insulin and glucose at 1, 2 and 3 hours. She will be fitted with a 24-hour blood pressure monitor.

Week 8b Visit: Approximately 6 Hours: Subject Will Fast from 9.00 p. m. the Prior Night Except for Water.

The subject will come to this visit fasting from 9.00 p.m. the prior night. She will return her 24-hour blood pressure monitor. She will have a 5-hour testing of her metabolic rate. Following the baseline testing, two naringenin capsules containing 310 mg of naringenin in a 1 g extract of sweet orange (Citrus sinensis) and two capsules containing 6 mg beta-carotene will be administered. After two hours of metabolic rate measurements she will be asked to drink the nutritional supplement (Boost) and have her metabolic rate measured for another 2 hours. She will return her bottle of naringenin capsules and beta carotene capsules with any remaining capsules collected for pill counts to determine compliance. She will be asked about adverse events. This will complete the study.

Procedures

Questionnaire, brief physical exam, questioning about adverse events and recording of height, weight, blood pressure and pulse rate: These procedures have no known risks and done as safety measures with weight and blood pressure also being measures of efficacy.

Blood testing: The insulin, CBC, oral glucose tolerance test and Chemistry panel are safety measures, because the subject has diabetes. The proteomics analysis will be conducted to identify the proteins regulated in response to the intervention. Drawing blood from an arm vein can involve the discomfort of a needle being placed in an arm vein and the possibility of bruising. In rare instances, fainting or infection can result. Trained technicians using aseptic technique with access to chairs that lie flat will minimize these risks.

Metabolic rate: The subject will lie quietly in bed for an hour and metabolic rate will be measured in the last 30 minutes of the first hour. She will then be given two 155 mg naringenin capsules and two 3 mg beta carotene capsules orally. She will have her metabolic rate measured during the last 30 minutes of each hour for 2 hours. The purpose of the metabolic rate testing is to determine if naringenin and beta carotene increase metabolic rate acutely or after 8 weeks. After 2 hours the subject will drink a 795 Kcal nutritional supplement (12 ounces Boost) metabolic rate will be measure during the last 30 minutes of the next 2 hours. An increase in metabolic rate can be associated with weight loss so weight will be measured monthly. The metabolic rate measurement involves having a plastic hood over ones face while the oxygen consumed and the carbon dioxide given off are measured. There are no known risks to measurement of metabolic rate.

Citrus Sinensis Extract and Beta Carotene: Citrus sinensis is sweet orange and a common food. There is no known risk to consuming an extract of sweet orange unless the person consuming the extract has a known allergy to citrus fruit. Beta carotene is a precursor of vitamin A and the amounts being given are within the range of beta carotene indicated for vitamin supplementation 6 to 15 mg per day. There are no risks known that are associated with this amount of beta carotene.

Glucose Tolerance Test: The glucose given with the oral glucose tolerance test is sweet and gives some people an upset feeling in their stomach. The test also can raise blood sugar, but is a medical test that is safe.

24-hour Ambulatory Blood Pressure Monitoring: A blood pressure cuff is attached to the upper arm on one side of the body and a tube runs from it to a small pack attached to the waist. The blood pressure monitor automatically inflates and takes the blood pressure every thirty minutes during the day and every hour during the night. The person goes about their normal activities and the monitor is worn the entire 24 hours except during bathing. There are no risks associated with the 24 hour blood pressure monitor testing.

Provisions to Monitor the Data to Ensure the Safety of Subjects

This is a minimal risk study. Naringenin is a component of a food (sweet orange), and the extract has no known risk in people who have no allergy to citrus fruit. The amount of blood taken during this study is about two tablespoonsful, an amount within the range of a minimal risk study. Despite this apparent safety, the subject will be monitored with measures of blood pressure, pulse rate, safety blood testing and recording of adverse events. The coordinator and principal investigator will discuss any adverse events to ensure safety of the subject.

Withdrawal of Subjects

Subjects can be withdrawn from the research without their consent, if in the determination of the principal investigator, remaining in the study is not in the best medical interest of the subject. The subject can withdraw from the research study at any time. If subject chooses to withdraw or not participate in the study, any health benefits to which the subject is entitled will not be affected in any manner.

Risks of Subjects

The risks of drawing blood include the discomfort of a needle going into an arm vein, bruising and rarely fainting or infection. Trained technicians using aseptic technique will minimize these risks. There are no known risks to the beta carotene or the extract of sweet orange which is a food, unless the subject has an allergy to citrus fruit.

Benefits to Subjects

Without wishing to be bound by theory, benefits that an individual subject can experience is knowledge about their metabolic rate and weight loss.

### References Cited in this Example

1. Rebello C J, Beyl R A, Lertora J J L, et al. Safety and pharmacokinetics of naringenin: A randomized, controlled, single-ascending-dose clinical trial. Diabetes Obes Metab 2020; 22(1):91-98.
2. Murugesan N, Woodard K, Ramaraju R, Greenway F L, Coulter A A, Rebello C J. Naringenin Increases Insulin Sensitivity and Metabolic Rate: A Case Study. J Med Food. 2019.

### Example 8

Fold Induction of Genes Upregulated by NRBC after 7d Treatment

| hgnc_symbol | description | fold | padj | meanCtrl | meanTreat |
|---|---|---|---|---|---|
| | brown/beige genes induced | | | | |
| | by PPAR activators | | | | |
| CIDEA | cell death inducing DFFA like | 12.4903419 | 4.04E−06 | 1.30922523 | 16.3526708 |
| | effector a [Source:HGNC | | | | |
| | Symbol;Acc:HGNC:1976] | | | | |
| CITED1 | Cbp/p300 interacting | 4.32598093 | 1.37E−11 | 13.4608798 | 58.2315093 |
| | transactivator with Glu/Asp rich | | | | |
| | carboxy-terminal domain 1 | | | | |
| | [Source:HGNC | | | | |
| | Symbol;Acc:HGNC:1986] | | | | |
| P11N4 | perilipin 4 [Source:HGNC | 4.06277867 | 4.16E−49 | 4473.79765 | 18176.0497 |
| | Symbol;Acc:HGNC:29393] | | | | |
| P11N5 | perilipin 5 [Source:HGNC | 3.03984082 | 1.39E−11 | 35.2367494 | 107.114109 |
| | Symbol;Acc:HGNC:33196] | | | | |
| P11N2 | perilipin 2 [Source:HGNC | 1.82666128 | 2.56E−13 | 18189.4449 | 33225.9547 |
| | Symbol;Acc:HGNC:248] | | | | |
| ELOVL3 | ELOVL fatty acid elongase 3 | 2.3909164 | 5.38E−07 | 67.5698082 | 161.553763 |
| | [Source:HGNC | | | | |
| | Symbol;Acc:HGNC:18047] | | | | |
| ELOVL6 | ELOVL fatty acid elongase 6 | 2.32550006 | 5.71E−13 | 3856.52166 | 8968.34133 |
| | [Source:HGNC | | | | |
| | Symbol;Acc:HGNC:15829] | | | | |
| ELOVL5 | ELOVL fatty acid elongase 5 | 2.21626314 | 2.46E−26 | 5238.49135 | 11609.8753 |
| | [Source:HGNC | | | | |
| | Symbol;Acc:HGNC:21308] | | | | |
| PDK4 | pyruvate dehydrogenase kinase | 4.24456627 | 1.02E−80 | 538.134027 | 2284.14554 |
| | 4 [Source:HGNC | | | | |
| | Symbol; Acc:HGNC:8812] | | | | |
| GK | glycerol kinase [Source:HGNC | 2.43089463 | 0.00542276 | 10.5204253 | 25.5740452 |
| | Symbol;Acc:HGNC:4289] | | | | |
| FABP7 | fatty acid binding protein 7 | 5.98345205 | 2.32E−09 | 39.802127 | 238.154118 |
| | [Source:HGNC | | | | |
| | Symbol;Acc:HGNC:3562] | | | | |
| FABP4 | fatty acid binding protein 4 | 5.55930631 | 2.42E−89 | 12514.8686 | 69573.9878 |
| | [Source:HGNC | | | | |
| | Symbol;Acc:HGNC:3559] | | | | |
| FABP3 | fatty acid binding protein 3 | 2.54386969 | 2.65E−14 | 59.690539 | 151.844953 |
| | [Source:HGNC | | | | |
| | Symbol;Acc:HGNC:3557] | | | | |
| FABP5 | fatty acid binding protein 5 | 1.92454569 | 1.10E−05 | 658.693715 | 1267.68615 |
| | [Source:HGNC | | | | |
| | Symbol;Acc:HGNC:3560] | | | | |
| | adipokines | | | | |
| CXCL14 | C-X-C motif chemokine ligand 14 | 1.70759662 | 0.06125503 | 24.1131991 | 41.1756174 |
| | [Source:HGNC | | | | |
| | Symbol;Acc:HGNC:10640] | | | | |
| FNDC4 | fibronectin type III domain | 1.71049735 | 3.55E−08 | 522.327999 | 893.440658 |
| | containing 4 [Source:HGNC | | | | |
| | Symbol;Acc:HGNC:20239] | | | | |
| ADIPOQ | adiponectin, C1Q and collagen | 4.28909127 | 1.55E−32 | 1304.94062 | 5597.00944 |
| | domain containing | | | | |
| | [Source:HGNC | | | | |
| | Symbol;Acc:HGNC:13633]\“” | | | | |
| CYP4F11 | cytochrome P450 family 4 | 14.0050733 | 2.99E−05 | 0.46416116 | 6.50061113 |
| | subfamily F member 11 | | | | |
| | [Source:HGNC | | | | |
| | Symbol;Acc:HGNC:13265] | | | | |
| EPHX1 | epoxide hydrolase 1 | 1.65415145 | 3.15E−06 | 1136.86493 | 1880.54678 |
| | [Source:HGNC | | | | |
| | Symbol;Acc:HGNC:3401] | | | | |
| EPHX2 | epoxide hydrolase 2 | 1.60153311 | 0.00062126 | 74.5710934 | 119.428076 |
| | [Source:HGNC | | | | |
| | SymbohAcc:HGNC:3402] | | | | |
| GDF11 | growth differentiation factor 11 | 2.22810965 | 2.56E−24 | 385.487202 | 858.907752 |
| | [Source:HGNC | | | | |
| | Symbol;Acc:HGNC:4216] | | | | |
| NAMPT | nicotinamide | 2.06598264 | 3.44E−23 | 601.135532 | 1241.93557 |
| | phosphoribosyltransferase | | | | |
| | [Source:HGNC | | | | |
| | Symbol;Acc:HGNC:30092] | | | | |
| NAPRT | nicotinate | 2.37464591 | 3.10E−22 | 82.7022309 | 196.388514 |
| | phosphoribosyltransferase | | | | |
| | [Source:HGNC | | | | |
| | Symbol;Acc:HGNC:30450] | | | | |
| ANG PTL4 | angiopoietin like 4 | 3.0897186 | 1.42E−34 | 641.344982 | 1981.57552 |
| | [Source:HGNC | | | | |
| | Symbol;Acc:HGNC:16039] | | | | |
| NMB | neuromedin B [Source:HGNC | 2.70311814 | 1.94E−30 | 1227.78425 | 3318.84587 |
| | Symbol;Acc:HGNC:7842] | | | | |
| POMC | proopiomelanocortin | 1.65768078 | 6.02E−06 | 55.2508406 | 91.5882568 |
| | [Source:HGNC | | | | |
| | Symbol;Acc:HGNC:9201] | | | | |
| S100B | S100 calcium binding protein B | 2.47177887 | 5.04E−11 | 108.708732 | 268.703946 |
| | [Source:HGNC | | | | |
| | Symbol;Acc:HGNC:10500] | | | | |
| LEP | leptin | 0.58481845 | 0.03765009 | 891.068 | 521.113004 |
| | thermogenesis | | | | |
| CKMT1A | \creatine kinase, mitochondrial | 5.07689935 | 3.66E−05 | 19.6789573 | 99.9080853 |
| | 1A [Source:HGNC | | | | |
| | Symbol;Acc:HGNC:31736]\“” | | | | |
| CKMT1B | \creatine kinase, mitochondrial | 3.92651718 | 3.46E−06 | 31.8162314 | 124.926979 |
| | 1B [Source:HGNC | | | | |
| | Symbol;Acc:HGNC:1995]\“” | | | | |
| CKMT2 | \creatine kinase, mitochondrial 2 | 3.82769832 | 5.12E−18 | 146.024882 | 558.939196 |
| | [Source:HGNC | | | | |
| | Symbol;Acc:HGNC:1996]\“” | | | | |
| UCP1 | uncoupling protein 1 | 16.713478 | 7.02E−12 | 1.86512242 | 31.1726825 |
| | [Source:HGNC | | | | |
| | Symbol;Acc:HGNC:12517] | | | | |
| UCP2 | uncoupling protein 2 | 3.35845942 | 2.53E−31 | 1290.48446 | 4334.03971 |
| | [Source:HGNC | | | | |
| | Symbol;Acc:HGNC:12518] | | | | |
| GPD1 | glycerol-3-phosphate | 3.27155297 | 1.76E−25 | 3971.50525 | 12992.9898 |
| | dehydrogenase 1 [Source:HGNC | | | | |
| | SymbolAcc:HGNC:4455] | | | | |
| GPD2 | glycerol-3-phosphate | 1.12138681 | 0.32342042 | 308.0199 | 345.409452 |
| | dehydrogenase 2 [Source:HGNC | | | | |
| | Symbol;Acc:HGNC:4456] | | | | |
| PM20D1 | peptidase M20 domain | 15.685394 | 2.17E−07 | 8.00551479 | 125.569653 |
| | containing 1 [Source:HGNC | | | | |
| | SymbolAcc:HGNC:26518] | | | | |
| CPT1B | carnitine palmitoyltransferase | 7.03004232 | 2.71E−08 | 3.12600075 | 21.9759176 |
| | 1B [Source:HGNC | | | | |
| | Symbol;Acc:HGNC:2329] | | | | |
| AlFM2 | apoptosis inducing factor | 1.96243444 | 1.70E−29 | 1078.99584 | 2117.45859 |
| | mitochondria associated 2 | | | | |
| | [Source:HGNC | | | | |
| | Symbol;Acc:HGNC:21411] | | | | |
| GK | glycerol kinase [Source:HGNC | 2.43089463 | 0.00542276 | 10.5204253 | 25.5740452 |
| | Symbol;Acc:HGNC:4289] | | | | |
| RXRG | retinoid X receptor gamma | 9.9683797 | 8.17E−19 | 4.20697239 | 41.9366982 |
| | [Source:HGNC | | | | |
| | Symbol;Acc:HGNC:10479] | | | | |
| IRX3 | iroquois homeobox 3 | 0.73269637 | 0.00089707 | 927.593861 | 679.644656 |
| Claussnitzer | [Source:HGNC | | | | |
| NEJM | Symbol;Acc:HGNC:14360] | | | | |
| IRX5 | iroquois homeobox 5 | 0.77345719 | 0.01532512 | 353.245545 | 273.220307 |
| | [Source:HGNC | | | | |
| | Symbol;Acc:HGNC:14361] | | | | |
| | Lipolysis | | | | |
| LIPE (HSL) | \lipase E, hormone sensitive | 3.16316646 | 2.52E−31 | 1777.02364 | 5621.02159 |
| | type [Source:HGNC | | | | |
| | Symbol;Acc:HGNC:6621]\“” | | | | |
| MGLL | monoglyceride lipase | 1.70183565 | 7.67E−22 | 2031.35948 | 3457.03998 |
| | [Source:HGNC | | | | |
| | Symbol;Acc:HGNC:17038] | | | | |
| PNPLA2 | patatin like phospholipase | 2.3038064 | 7.36E−22 | 4795.68858 | 11048.338 |
| | domain containing 2 | | | | |
| | [Source:HGNC | | | | |
| | Symbol;Acc:HGNC:30802] | | | | |
| AQP7 glycerol | aquaporin 7 [Source:HGNC | 3.5405188 | 4.26E−20 | 448.687725 | 1588.58732 |
| channel | Symbol;Acc:HGNC:640] | | | | |
| PRKAR2B | protein kinase cAMP-dependent | 2.46878364 | 3.52E−20 | 767.261454 | 1894.20253 |
| | type II regulatory subunit beta | | | | |
| | [Source:HGNC | | | | |
| | Symbol;Acc:HGNC:9392] | | | | |
| PRKACA | protein kinase cAMP-activated | 1.44365332 | 1.15E−19 | 1287.03756 | 1858.03605 |
| | catalytic subunit alpha | | | | |
| | [Source:HGNC | | | | |
| | Symbol;Acc:HGNC:9380] | | | | |
| PRKAR2A | protein kinase cAMP-dependent | 1.40059635 | 1.99E−12 | 4080.36084 | 5714.93848 |
| | type II regulatory subunit alpha | | | | |
| | [Source:HGNC | | | | |
| | Symbol;Acc:HGNC:9391] | | | | |

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A composition comprising a therapeutically effective amount of at least one flavonoid and a therapeutically effective amount of at least one carotenoid.

2. The composition of claim 1, further comprising a sufficient amount of a pharmaceutically acceptable carrier.

3. The method of claim 1, wherein the composition comprises about 150 mg to about 900 mg of at least one flavonoid.

4. The method of claim 1, wherein the composition comprises about 1 mg to about 12 mg of at least one carotenoid.

5. The composition of claim 1, wherein the flavonoid is naringenin.

6. The composition of claim 1, wherein the at least one carotenoid is selected from the group consisting of beta carotene, lycopene, or lutein.

7. The composition of claim 1, wherein the at least one carotenoid is beta carotene.

8. The composition of claim 1, wherein the composition further comprises one or more additional active agents.

9. The composition of claim 1, wherein the additional active agent comprises an anti-obesity agent.

10. The composition of claim 2, wherein the composition is provided as an injectable solution, an oral dose, a topical cream, a topical gel, or a medical food.

11. A method for treating a subject afflicted with a metabolic disorder, the method comprising administering to the subject a therapeutically effective amount of a composition comprising at least one flavonoid and at least one carotenoid.

12. The method of claim 11, wherein the composition comprises about 150 mg to about 900 mg of at least one flavonoid.

13. The method of claim 11, wherein the composition comprises about 1 mg to about 12 mg of at least one carotenoid.

14. The method of claim 11, wherein the flavonoid is naringenin.

15. The method of claim 11, wherein the at least one carotenoid is selected from the group consisting of beta carotene, lycopene, or lutein.

16. The method of claim 11, wherein the at least one carotenoid is beta carotene.

17. The method of claim 11, wherein the metabolic disorder comprises obesity, insulin resistance, type 2 diabetes, metabolic syndrome, and non-alcoholic steohepatis.

18. The method of claim 11, wherein the composition is administered as a topical cream, administered as a topic gel, administered as a medical food, or administered as an injectable.

19. The method of claim 18, wherein the topical cream is administered locally.

20. A method of converting white fat to brown fat in a subject, the method comprising administering to the subject a therapeutically effective amount of a composition comprising at least one flavonoid and at least one carotenoid.

21. The method of claim 20, wherein the composition comprises about 150 mg to about 900 mg of at least one flavonoid.

22. The method of claim 20, wherein the composition comprises about 1 mg to about 12 mg of at least one carotenoid.

23. The method of claim 20, wherein the flavonoid is naringenin.

24. The method of claim 20, wherein the at least one carotenoid is selected from the group consisting of beta carotene, lycopene, or lutein.

25. The method of claim 20, wherein the at least one carotenoid is beta carotene.

26. The method of claim 20, wherein the composition is administered as a topical cream, administered as a topic gel, administered as a medical food, administered as an oral dose, or administered as an injectable.

27. The method of claim 26, wherein the topical cream is administered locally.

28. A method for local fat reduction, the method comprising administering to a site on a subject a therapeutically effective amount of a composition comprising at least one flavonoid and at least one carotenoid.

29. The method of claim 28, wherein the composition comprises about 150 mg to about 900 mg of at least one flavonoid.

30. The method of claim 28, wherein the composition comprises about 1 mg to about 12 mg of at least one carotenoid.

31. The method of claim 28, wherein the flavonoid is naringenin.

32. The method of claim 28, wherein the at least one carotenoid is selected from the group consisting of beta carotene, lycopene, or lutein.

33. The method of claim 28, wherein the at least one carotenoid is beta carotene.

34. The method of claim 21, wherein the composition is administered as a topical cream, administered as a topic gel, or administered as an injectable.
